SEQUENCE LISTING (16 sequences)

SEQ ID NO: 1 - protein, 503 aa, Homo sapiens
MTLGSPRKGLLMLLMALVTQGDPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQLALILGPVLALLALVALGVLGLWHVRRRQEKQRGLHSELGESSLILKASEQGDSMLGDLLDSDCTTGSGSGLPFLVQRTVARQVALVECVGKGRYGEVWRGLWHGESVAVKIFSSRDEQSWFRETEIYNTVLLRHDNILGFIASDMTSRNSSTQLWLITHYHEHGSLYDFLQRQTLEPHLALRLAVSAACGLAHLHVEIFGTQGKPAIAHRDFKSRNVLVKSNLQCCIADLGLAVMHSQGSDYLDIGNNPRVGTKRYMAPEVLDEQIRTDCFESYKWTDIWAFGLVLWEIARRTIVNGIVEDYRPPFYDVVPNDPSFEDMKKVVCVDQQTPTIPNRLAADPVLSGLAQMMRECWYPNPSARLTALRIKKTLQKISNSPEKPKVIQ

SEQ ID NO: 2 - DNA, 1512 bp, Homo sapiens
atgaccttgggctcccccaggaaaggccttctgatgctgctgatggccttggtgacccagggagaccctgtgaagccgtctcggggcccgctggtgacctgcacgtgtgagagcccacattgcaaggggcctacctgccggggggcctggtgcacagtagtgctggtgcgggaggaggggaggcacccccaggaacatcggggctgcgggaacttgcacagggagctctgcagggggcgccccaccgagttcgtcaaccactactgctgcgacagccacctctgcaaccacaacgtgtccctggtgctggaggccacccaacctccttcggagcagccgggaacagatggccagctggccctgatcctgggccccgtgctggccttgctggccctggtggccctgggtgtcctgggcctgtggcatgtccgacggaggcaggagaagcagcgtggcctgcacagcgagctgggagagtccagtctcatcctgaaagcatctgagcagggcgacagcatgttgggggacctcctggacagtgactgcaccacagggagtggctcagggctccccttcctggtgcagaggacagtggcacggcaggttgccttggtggagtgtgtgggaaaaggccgctatggcgaagtgtggcggggcttgtggcacggtgagagtgtggccgtcaagatcttctcctcgagggatgaacagtcctggttccgggagactgagatctataacacagtgttgctcagacacgacaacatcctaggcttcatcgcctcagacatgacctcccgcaactcgagcacgcagctgtggctcatcacgcactaccacgagcacggctccctctacgactttctgcagagacagacgctggagccccatctggctctgaggctagctgtgtccgcggcatgcggcctggcgcacctgcacgtggagatcttcggtacacagggcaaaccagccattgcccaccgcgacttcaagagccgcaatgtgctggtcaagagcaacctgcagtgttgcatcgccgacctgggcctggctgtgatgcactcacagggcagcgattacctggacatcggcaacaacccgagagtgggcaccaagcggtacatggcacccgaggtgctggacgagcagatccgcacggactgctttgagtcctacaagtggactgacatctgggcctttggcctggtgctgtgggagattgcccgccggaccatcgtgaatggcatcgtggaggactatagaccacccttctatgatgtggtgcccaatgaccccagctttgaggacatgaagaaggtggtgtgtgtggatcagcagacccccaccatccctaaccggctggctgcagacccggtcctctcaggcctagctcagatgatgcgggagtgctggtacccaaacccctctgcccgactcaccgcgctgcggatcaagaagacactacaaaaaattagcaacagtccagagaagcctaaagtgattcaatag

SEQ ID NO: 3 - protein, 328 aa, Artificial sequence
  Recombinant protein
DPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQLATGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 1075 bp, Artificial sequence
  Recombinant DNA
gctagcaccatggatgcaatgaagagagggctctgctgtgtgctgctgctgtgtggagcagtcttcgtttcgcccggcgccgaccctgtgaagccgtctcggggcccgctggtgacctgcacgtgtgagagcccacattgcaaggggcctacctgccggggggcctggtgcacagtagtgctggtgcgggaggaggggaggcacccccaggaacatcggggctgcgggaacttgcacagggagctctgcaggggccgccccaccgagttcgtcaaccactactgctgcgacagccacctctgcaaccacaacgtgtccctggtgctggaggccacccaacctccttcggagcagccgggaacagatggccagctggccaccggtggtggaactcacacatgcccaccgtgcccagcacctgaagccctgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggccccttcttcctctacagcaagctcaccgtggacaagagcaggtggcagcaggggaacgtcttctcatgctccgtgatgcatgaggctctgcacaaccactacacgcagaagagcctctccctgtctccgggtaaatgaggaattc

SEQ ID NO: 5 - protein, 352 aa, Artificial sequence
  Recombinant protein
MDAMKRGLCCVLLLCGAVFVSPGADPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQLATGGGTHTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGPFFLYSKLTVDKSRWQQGNVFSCSVMHEALHNHYTQKSLSLSPGK

SEQ ID NO: 6 - protein, 225 aa, Homo sapiens
  misc_feature (43)..(43): residue may optionally be mutated to alanine
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGPFFLYSKLTVDKSRWQQGNVFSCSVMHEALHNHYTQKSLSLSPGK

SEQ ID NO: 7 - protein, 21 aa, Artificial sequence
  Recombinant leader peptide
MKFLVNVALVFMVVYISYIYA

SEQ ID NO: 8 - protein, 22 aa, Artificial sequence
  Recombinant leader peptide
MDAMKRGLCCVLLLCGAVFVSP

SEQ ID NO: 9 - protein, 21 aa, Artificial sequence
  Recombinant leader peptide
MTLGSPRKGLLMLLMALVTQG

SEQ ID NO: 10 - protein, 180 aa, Homo sapiens
MLRVLVGAVLPAMLLAAPPPINKLALFPDKSAWCEAKNITQIVGHSGCEAKSIQNRACLGQCFSYSVPNTFPQSTESLVHCDSCMPAQSMWEIVTLECPGHEEVPRVDKLVEKILHCSCQACGKEPSHEGLSVYVQGEDGPGSQPGTHPHPHPHPHPGGQTPEPEDPPGAPHTEEEGAED

SEQ ID NO: 11 - DNA, 2003 bp, Homo sapiens
gccgagcctcctggggcgcccgggcccgcgacccccgcacccagctccgcaggaccggcgggcgcgcgcgggctctggaggccacgggcatgatgcttcgggtcctggtgggggctgtcctccctgccatgctactggctgccccaccacccatcaacaagctggcactgttcccagataagagtgcctggtgcgaagccaagaacatcacccagatcgtgggccacagcggctgtgaggccaagtccatccagaacagggcgtgcctaggacagtgcttcagctacagcgtccccaacaccttcccacagtccacagagtccctggttcactgtgactcctgcatgccagcccagtccatgtgggagattgtgacgctggagtgcccgggccacgaggaggtgcccagggtggacaagctggtggagaagatcctgcactgtagctgccaggcctgcggcaaggagcctagtcacgaggggctgagcgtctatgtgcagggcgaggacgggccgggatcccagcccggcacccaccctcacccccatccccacccccatcctggcgggcagacccctgagcccgaggacccccctggggccccccacacagaggaagagggggctgaggactgaggcccccccaactcttcctcccctctcatccccctgtggaatgttgggtctcactctctggggaagtcaggggagaagctgaagcccccctttggcactggatggacttggcttcagactcggacttgaatgctgcccggttgccatggagatctgaaggggcggggttagagccaagctgcacaatttaatatattcaagagtggggggaggaagcagaggtcttcagggctctttttttggggggggggtggtctcttcctgtctggcttctagagatgtgcctgtgggagggggaggaagttggctgagccattgagtgctgggggaggccatccaagatggcatgaatcgggctaaggtccctgggggtgcagatggtactgctgaggtcccgggcttagtgtgagcatcttgccagcctcaggcttgagggagggctgggctagaaagaccactggcagaaacaggaggctccggccccacaggtttccccaaggcctctcaccccacttcccatctccagggaagcgtcgccccagtggcactgaagtggccctccctcagcggaggggtttgggagtcaggcctgggcaggaccctgctgactcgtggcgcgggagctgggagccaggctctccgggcctttctctggcttccttggcttgcctggtgggggaaggggaggaggggaagaaggaaagggaagagtcttccaaggccagaaggagggggacaaccccccaagaccatccctgaagacgagcatccccctcctctccctgttagaaatgttagtgccccgcactgtgccccaagttctaggccccccagaaagctgtcagagccggccgccttctcccctctcccagggatgctctttgtaaatatcggatgggtgtgggagtgaggggttacctccctcgccccaaggttccagaggccctaggcgggatgggctcgctgaacctcgaggaactccaggacgaggaggacatgggacttgcgtggacagtcagggttcacttgggctctctctagctccccaattctgcctgcctcctccctcccagctgcactttaaccctagaaggtggggacctggggggagggacagggcaggcgggcccatgaagaaagcccctcgttgcccagcactgtctgcgtctgctcttctgtgcccagggtggctgccagcccactgcctcctgcctggggtggcctggccctcctggctgttgcgacgcgggcttctggagcttgtcaccattggacagtctccctgatggaccctcagtcttctcatgaataaattccttcaacgccaaaaaaaaaaaaaaaaaaaaaaaaaaaaaaaaaaaaaaaaaa

SEQ ID NO: 12 - protein, 111 aa, Artificial sequence
  Recombinant protein
RSAGAGSHCQKTSLRVNFEDIGWDSWIIAPKEYEAYECKGGCFFPLADDVTPTKHAIVQTLVHLKFPTKVGKACCVPTKLSPISVLYKDDMGVPTLKYHYEGMSVAECGCR

SEQ ID NO: 13 - protein, 108 aa, Artificial sequence
  Recombinant protein
NAKGNYCKRTPLYIDFKEIGWDSWIIAPPGYEAYECRGVCNYPLAEHLTPTKHAIIQALVHLKNSQKASKACCVPTKLEPISILYLDKGVVTYKFKYEGMAVSECGCR

SEQ ID NO: 14 - protein, 328 aa, Homo sapiens
DPVKPSRGPLVTCTCESPHCKGPTCRGAWCTVVLVREEGRHPQEHRGCGNLHRELCRGRPTEFVNHYCCDSHLCNHNVSLVLEATQPPSEQPGTDGQLATGGGTHTCPPCPAPEALGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGPFFLYSKLTVDKSRWQQGNVFSCSVMHEALHNHYTQKSLSLSPGK

SEQ ID NO: 15 - protein, 120 aa, Homo sapiens
RSAGAGSHCQKTSLRVNFEDIGWDSWIIAPKEYEAYECKGGCFFPLADDVTPTKHAIVQTLNAKGNYCKRTPLYIDFKEIGWDSWIIAPPGYEAYECRGVCNYPLAEHLTPTKHAIIQAL

SEQ ID NO: 16 - protein, 99 aa, Homo sapiens
VHLKFPTKVGKACCVPTKLSPISVLYKDDMGVPTLKYHYEGMSVAECGCRVHLKNSQKASKACCVPTKLEPISILYLDKGVVTYKFKYEGMAVSECGCR

(19) United States
(12) Patent Application Publication
(10) Pub. No.: US 2013/0202594 A1
(43) Pub. Date: Aug. 8, 2013

(54) ALK1 Antagonists and Their Uses in Treating Renal Cell Carcinoma

(71) Applicants: Rupal S. BHATT, Roslindale, MA (US); Ravindra Kumar, Acton, MA (US); James W. Mier, Brookline, MA (US); Robert Pearsall, Woburn, MA (US); Matthew Sherman, Newton, MA (US); Nicolas Solban, Brighton, MA (US)

(72) Inventors: Rupal S. BHATT, Roslindale, MA (US); Ravindra Kumar, Acton, MA (US); James W. Mier, Brookline, MA (US); Robert Pearsall, Woburn, MA (US); Matthew Sherman, Newton, MA (US); Nicolas Solban, Brighton, MA (US)

(21) Appl. No.: 13/757,672

(22) Filed: Feb. 1, 2013

Related U.S. Application Data
(60) Provisional application No. 61/593,864, filed on Feb. 2, 2012.
(60) Provisional application No. 61/597,124, filed on Feb. 9, 2012.

(51) Int. Cl. A61K 38/45; A61K 31/404; A61K 31/44; A61K 31/436; A61K 31/4439; A61K 31/4709; A61K 31/517; A61K 39/395; A61K 31/506

(52) U.S. Cl. CPC A61K 38/45; A61K 39/3955; A61K 31/404; A61K 31/44; A61K 31/506; A61K 31/4439; A61K 31/4709; A61K 31/517; A61K 31/436
USPC 424/134.1; 424/94.5; 424/139.1

(57) ABSTRACT

In certain aspects, the present disclosure relates to the insight that a polypeptide comprising a ligand-binding portion of the extracellular domain of activin-like kinase I (ALK1) polypeptide may be used to inhibit tumor growth of renal cell carcinoma (RCC) in vivo. In additional aspects the disclosure relates to the insight that a polypeptide comprising a ligand-binding portion of the extracellular domain of ALK1 dramatically increases the ability of a standard of care receptor tyrosine kinase inhibitor to inhibit RCC tumor growth in vivo.

### REFERENCE TO SEQUENCE LISTING SUBMITTED ELECTRONICALLY

A Sequence Listing is submitted electronically via EFS-Web as an ASCII formatted sequence listing in a file named “3174_{—}0010002_SEQLIST.txt”, created on Feb. 1, 2013, and having a file size of 32,000 bytes which is filed concurrently with the present specification, claims, abstract and figures provided herewith. The sequence listing contained in this ASCII formatted document is part of the specification and is herein incorporated by reference in its entirety.

### CROSS-REFERENCE TO RELATED APPLICATIONS

This application claims the benefit of U.S. Provisional Application No. 61/593,864, filed Feb. 2, 2012, and U.S. Provisional Application No. 61/597,124, filed Feb. 9, 2012, each of which is herein incorporated by reference in its entirety.

### BACKGROUND

Renal Cell Carcinoma (RCC), accounts for up to 90% of all malignant kidney tumors and is the eighth most commonly diagnosed cancer in men and women in the U.S. The National Cancer Institute estimates that approximately 65,000 new cases of renal cancer will be diagnosed in the U.S. in 2012 and that approximately 13,600 deaths will result from renal cancer. Worldwide it is estimated that more than 200,000 new cases are diagnosed and more than 100,000 die from RCC each year. Both incidence and mortality of RCC are increasing worldwide.

RCC can often be cured through surgical removal of the tumor or kidney if diagnosed and treated when still localized to the kidney or immediate surrounding tissue. However, the probability of disease free survival significantly decreases as the cancer becomes vascularized and metastasizes to distant parts of the body. One third of RCC presents as metastatic disease, with a five-year survival rate of less than 10%.

Metastatic RCC (mRCC) historically has been insensitive to chemotherapy and hormonal therapy and until very recently, systemic treatment has been limited to non-specific immune-based cytokine therapy with interleukin 2 (IL-2) or interferon alpha (IFN-α). These therapies are associated with low rates of response and high rates of toxicity.

Research during the past decade has helped to elucidate genetic events associated with RCC tumorigenesis and advanced disease. In particular, the aberrant signaling of the vascular endothelial growth factor (VEGF), platelet derived growth factor (PDGF), and AKT/mTOR (mammalian target of rapamycin) signaling pathways both within tumor cells and between tumor cells and surrounding tissue (e.g., resident endothelial cells and pericytes) have been identified to play influential roles in driving RCC vascularization, cell survival, and tumor proliferation. The association of aberrancies in these pathways with RCC has in turn led to the development of a wave of therapies targeting key steps in the VEGF, PDGF and mTOR signaling pathways. In particular, since 2005, five agents that target the VEGF and PDGF pathway (i.e., sorafenib, sunitinib, bevacizumab, pazopanib, and axitinib) and two mTOR pathway-targeted therapies (i.e., temsirolimus and everolimus) have been approved by the FDA for advanced RCC indications.

With the exception of bevacizumab (a humanized antibody that binds VEGF, commonly known as AVASTIN®) the approved RCC therapies that target the VEGF pathway are small-molecule ATP-mimetic inhibitor compounds. These small molecule inhibitors act by binding the highly conserved ATP-binding catalytic site of receptor tyrosine kinases such as, VEGFR1, VEGFR2, and VEGFR3, and thereby blocking the intracellular signaling of the bound receptor. However, due in part to the highly conserved structure of the ATP-binding catalytic site amongst protein kinases, most small molecule receptor tyrosine kinase inhibitors also bind to and inhibit distinct unintended receptor tyrosine kinases, and sometimes even members of other kinase families. Such “off-target” action of receptor tyrosine kinase inhibitors frequently lead to adverse events and toxicities that limit the therapeutic applications and/or efficacy of the drug.

Sunitinib (commonly known as SUTENT®) is a multitarget receptor tyrosine kinase inhibitor that was initially developed as a small molecule inhibitor of the c-Met receptor tyrosine kinase. In addition to c-Met, sunitinib competitively inhibits activity of the VEGH1, VEGFR2, VEGFR3, PDGFRa, PDGFRb, flt-3, c-KIT (CD117), RET, and CSF-1R receptor tyrosine kinases. Sunitinib received approval as a first line therapy in treating advanced RCC after concluding pivotal trials demonstrating that sunitinib prolonged overall survival in patients with advanced disease by nearly five months compared to interferon-alpha (26.4 months vs. 21.8 months). Although modest, this improvement in patient survival has made sunitinib the new standard of care for treatment-naïve patients with advanced RCC. Sunitinib therapy is associated with significant side effects, as demonstrated by the requirement of dose reductions in 50% of the RCC patients in order to manage the significant toxicities associated with sunitinib.

Despite recent advances in RCC therapies, significant unmet need persists. Currently available therapies provide patients less than one year of survival without disease progression and are associated with significant toxicities. Moreover, adaptation of the tumor to the treatment frequently leads to the discontinuation of treatment and accelerated tumor growth.

### SUMMARY

The present disclosure provides antagonists of the activin-like kinase I (ALK1)-regulatory system and the use of such antagonists to treat renal cell carcinoma (RCC). In particular aspects, the RCC is clear cell renal cell carcinoma. In further aspects, the RCC is a TNM (Tumor/Mode/Metastasis classification) stage III disease. In additional aspects, the RCC is a TNM stage IV disease. In additional aspects, the RCC is found within the intrarenal veins. In other aspects, the RCC has invaded the renal sinus. In further aspects, the RCC has metastasized to the adrenal gland or to a lymph node. In further aspects, the RCC has metastasized to the lung, intra-abdominal lymph nodes, bone, brain, or liver.

As described herein, ALK1 is a receptor for the GDF5 (growth differentiation factor 5) group of ligands, which includes GDF6 and GDF7, and also for the BMP9 (bone morphogenetic protein 5) group of ligands, which includes BMP10. This disclosure demonstrates that signaling mediated by ALK1 and the ligands described above is involved in angiogenesis in vivo, and that the inhibition of this regulatory system has a potent anti-angiogenic effect.

The disclosure also demonstrates that the use of ALK1 regulatory system antagonists, such as an ALK1-Fc fusion protein, inhibits tumor growth in a human RCC xenograft animal model. The disclosure further demonstrates that an ALK1-Fc fusion protein antagonist of ALK1 significantly enhances the tumor growth inhibiting activity of sunitinib, a VEGF receptor tyrosine kinase inhibitor, when administered in combination with sunitinib in human RCC xenograft animal models. Thus, in certain aspects, the disclosure provides antagonists of the ALK1 regulatory system, including antagonists of the ALK1 receptor or one or more ALK1 ligands, for use in treating renal cell carcinoma. In particular aspects, the ALK1 antagonist is an ALK1-Fc fusion protein (e.g., an ALK1-Fc fusion protein as described herein). In certain aspects, the disclosure provides antagonists of the ALK1 regulatory system, including antagonists of the ALK1 receptor or one or more of the ALK1 ligands, for use in treating renal cell carcinoma. In particular aspects, the renal cell carcinoma is clear cell renal cell carcinoma. In additional aspects, the renal cell carcinoma that is treated has invaded the renal sinus. In some aspects, the RCC is a TNM stage III disease. In additional aspects, the RCC is a TNM stage IV disease. In additional aspects, the RCC is found within the intrarenal veins. In other aspects, the RCC has invaded the renal sinus. In further aspects, the RCC has metastasized to the adrenal gland or to a lymph node. In further aspects, the RCC has metastasized to the lung, intra-abdominal lymph nodes, bone, brain, or liver.

In certain aspects, the disclosure provides polypeptides comprising a ligand binding portion of the extracellular domain of ALK1 (“ALK1 ECD polypeptides”) for use in inhibiting angiogenesis. In additional aspects, the disclosure provides polypeptides comprising ALK1 ECD polypeptides for use in treating RCC (e.g., clear cell renal cell carcinoma). While not wishing to be bound to any particular mechanism of action, it is expected that such polypeptides act by binding to ligands of ALK1 and inhibiting the ability of these ligands to interact with ALK1, as well as other receptors. In certain embodiments, an ALK1 ECD polypeptide comprises an amino acid sequence that is at least 70%, 80%, 90%, 95%, 96%, 97%, 98%, 99%, or 100% identical to the sequence of amino acids 22-118 of the human ALK1 sequence of SEQ ID NO:1. In certain embodiments, an ALK1 ECD polypeptide comprises an amino acid sequence that is at least 70%, 80%, 90%, 95%, 96%, 97%, 98%, 99%, or 100% identical to the sequence of amino acids 22-120 of the human ALK1 sequence of SEQ ID NO: 1. An ALK1 ECD polypeptide can be used as a small monomeric protein or in a dimerized form (e.g., expressed as an Fc fusion protein). An ALK1 ECD can also be fused to a second polypeptide portion to provide improved or desired properties, such as an improved ligand binding affinity, increased half-lite or greater ease of production or purification. Fusions to an Fc portion of an immunoglobulin or linkage to a polyoxyethylene moiety (e.g., polyethylene glycol) are particularly useful for increasing the serum half-life of the ALK1 ECD polypeptide during systemic administration (e.g., intravenous, intraarterial and intra-peritoneal administration).

As demonstrated herein, a systemically administered ALK1-Fc fusion protein has a potent tumor growth inhibiting effect when administered alone in a human RCC mouse xenograft model and dramatically increases sunitinib RCC tumor growth inhibition when systemically administered with sunitinib in the human RCC mouse xenograft models tested. In certain embodiments, an ALK1-Fc fusion protein comprises a polypeptide having an amino acid sequence that is at least 70%, 80%, 90%, 95%, 96%, 97%, 98%, 99%, or 100% identical to the sequence of amino acids 22-118 or 22-120 of SEQ ID NO:1, which polypeptide is fused, either with or without an intervening linker, to an Fc portion of an immunoglobulin, and wherein the ALK1-Fc fusion protein binds to an ALK1 ligand selected from GDF5 (e.g., having the sequence recited in Genbank Accession No. CAA56874), GDF6 (e.g., having the sequence recited in Genbank Accession No. AAH43222), GDF7 (e.g., having the sequence recited in Genbank Accession No. NP_{—}878248), BMP9 (e.g., having the sequence recited in Genbank Accession No. AF156891 AF188285 AK314956 BC069643 or BC074921) and BMP 10 (e.g., having the sequence recited in Genbank Accession No. 095393). In further aspects, the ALK1-Fc fusion protein binds to an ALK1 ligand selected from GDF5, GDF7 and BMP9 with a K_{D} of less than 1×10⁻⁷ M and binds to TGFβ-1 with a K_{D} of greater than 1×10⁻⁶ M. Fc portions of the Fc fusion protein are selected so as to be appropriate to the organism being treated and so as to exhibit the desired pharmacokinetic and pharmacodymamic properties. Optionally, the Fc portion is an Fc portion of a human IgG1. In a preferred embodiment, the ALK1-Fc fusion protein comprises amino acids 22-118 or 22-120 of SEQ ID NO:1. Optionally, the ALK1-Fc fusion protein comprises the amino acid sequence of SEQ ID NO: 3. Optionally, the ALK1-Fc fusion protein comprises the amino acid sequence of SEQ ID NO: 14. Optionally, the ALK1-Fc fusion protein is the protein produced by expression of the nucleic acid of SEQ ID NO:4 in a mammalian cell line, particularly a Chinese Hamster Ovary (CHO) cell line. ALK1-ECD polypeptides are formulated as pharmaceutical preparations that are substantially pyrogen free. The pharmaceutical preparation can be prepared for systemic delivery (e.g., intravenous, intraarterial or subcutaneous delivery) or local delivery.

In certain aspects, the disclosure addresses the difficulties in developing relatively homogeneous preparations of ALK1-Fc fusion protein for use in a therapeutic setting. As described herein, ALK1-Fc fusion proteins tend to aggregate into higher order multimers. The disclosure provides solutions to these difficulties and therefore provides pharmaceutical preparations comprising ALK1-Fc fusion proteins wherein such preparations are composed of at least 85%, 90%, 95%, 96%, 97%, 98%, or 99% dimeric ALK1-Fc fusion protein. Therefore, in certain aspects, the disclosure provides pharmaceutical preparations containing an ALK1-Fc fusion protein comprising: a polypeptide having an amino acid sequence that is at least 90%, 95%, 96% or 97% identical to the sequence of amino acids 22-118 or 22-120 of SEQ ID NO:1, which polypeptide is fused to an Fc portion of an immunoglobulin, and wherein the ALK1-Fc fusion protein binds to a ligand selected from GDF5, GDF6, GDF7, BMP9 and BMP 10. In further aspects, the ALK1-Fc fusion protein binds GDF5, GDF7 and BMP9 with a K_{D} of less than 1×10⁻⁷M and binds to TGFβ-1 with a K_{D} of greater than 1×10⁻⁶M and wherein at least 85%, 90%, 95%, 96%, 97%, 98%, or 99% of the ALK1-Fc fusion protein is present in a dimeric form.

The Fc portion of the ALK1-Fc fusion protein can be an Fc portion of a human IgG1 or another human immunoglobulin subclass, such as IgG2 or IgG3. In some aspects the ALK1-Fc fusion protein comprises the amino acid sequence of SEQ ID NO:3. In other aspects the ALK1-Fc fusion protein comprises the amino acid sequence of SEQ ID NO:14. In further aspects, the ALK1-Fc fusion protein is produced by the expression of the nucleic acid of SEQ ID NO:4 in a mammalian cell line, such as a Chinese Hamster Ovary (CHO) cell line. Such pharmaceutical preparations can be formulated with the objective of optimizing the desired properties of the ALK1-Fc fusion protein using known techniques and reagents.

The pharmaceutical preparations of the invention can be used for a variety of therapeutic purposes described herein, including inhibiting angiogenesis and treating RCC. In a particular aspect, the pharmaceutical preparations are used to treat clear cell renal cell carcinoma. In a further aspect, the pharmaceutical preparations are used to treat RCC in a mammal having previously received an RCC therapeutic agent. In another aspect the pharmaceutical preparations are used to treat a mammal that has RCC and that has undergone or is preparing to undergo a medical procedure to treat RCC. In a further aspect, the pharmaceutical preparations of the invention are used to treat advanced (metastatic) RCC. In additional aspects, the pharmaceutical preparations of the invention are used to inhibit angiogenesis and/or to treat a disease or disorder in which inhibiting angiogenesis is desirable.

In some embodiments, the ALK1-Fc pharmaceutical preparations and preparations comprising antibodies directed to ALK1 or one or more ligands of ALK1 (e.g., BMP9 and/or BMP10) are used in conjunction with an agent that inhibits angiogenesis. In some embodiments, the ALK1-Fc pharmaceutical preparations and preparations comprising antibodies directed to ALK1 or one or more ligands of ALK1 (e.g., BMP9 and/or BMP10) are used in conjunction with a VEGF signaling pathway antagonist (e.g., an antibody that binds VEGF (e.g., AVASTIN®), a VEGF receptor (e.g., VEGFR1, VEGFR2, and VEGFR3) and a VEGF receptor trap). In particular aspects, the pharmaceutical preparations comprise a VEGF receptor tyrosine kinase inhibitor. In further aspects the VEGF receptor tyrosine kinase inhibitor is an agent selected from sunitinib (SUTENT®), sorafenib (NEXAVAR®), pazopanib (VOTRIENT®), axitinib (INLYTA®), tivozanib and vandetanib.

In certain aspects, the disclosure provides methods for treating renal cell carcinoma in a mammal by administering to a mammal having RCC, an ALK1 ECD polypeptide. In a further aspect, the disclosure provides a method of treating RCC in a mammal, comprising administering to a mammal that has RCC an effective amount of an activin-like kinase I (ALK1)-Fc fusion protein and a VEGF receptor tyrosine kinase inhibitor. In one aspect, the RCC to be Leafed is a clear cell renal cell carcinoma. In another aspect, the RCC to be treated has invaded the renal sinus. In some aspects, the RCC is a TNM stage III disease. In additional aspects, the RCC is a TNM stage IV disease. In additional aspects, the RCC is found within the intrarenal veins. In further aspects, the RCC has metastasized to the adrenal gland or to a lymph node. In further aspects, the RCC has metastasized to the lung, intra-abdominal lymph nodes, bone, brain, or liver.

In certain aspects, the ALK1-Fc fusion protein administered according to a method of the invention comprises a polypeptide having an amino acid sequence that is at least 80%, 90%, 95%, 96%, 97%, 98%, 99% or 100% identical to the sequence of amino acids 22-118 or 22-120 of SEQ ID NO:1, which polypeptide is fused to an Fc portion of an immunoglobulin, and wherein the ALK1-Fc fusion protein binds to an ALK-ligand selected from GDF5, GDF6, GDF7, BMP9 and BMP10. In further aspects, the ALK1-Fc fusion protein binds TGFβ-1 with a K_{D} of greater than 1×10⁻⁶ M. Optionally, the ALK1-Fc fusion protein has a sequence of SEQ ID NO:3. In an alternative option, the ALK1-Fc fusion protein has a sequence of SEQ ID NO:14. The ALK1 ECD polypeptide may be delivered locally or systemically (e.g., intravenously, intraarterially or subcutaneously).

In a further aspect, the VEGF receptor tyrosine kinase inhibitor administered with the ALK1-Fc fusion protein is an agent selected from sunitinib (SUTENT®), sorafenib (NEXAVAR®), pazopanib (VOTRIENT®), axitinib (INLYTA®), tivozanib and vandetanib.

In another aspect, the disclosure provides a method of treating RCC in a mammal, comprising administering to a mammal that has RCC an effective amount of an activin-like kinase I (ALK1)-Fc, a VEGF receptor tyrosine kinase inhibitor, and a mammalian target of rapamycin (mTOR) inhibitor. In a further aspect an ALK1-Fc fusion protein and VEGF receptor tyrosine kinase inhibitor are administered with the mTOR-targeted inhibitor everolimus or temsirolimus. In other aspects, the mTOR inhibitor is an agent selected from: WYE354, YE132 (Pfizer), PP30 and PP242, AZD8055, OSI-027, Torin1, BEZ235, XL765, GDC-0980, PF-04691502 and PF-05212384.

In one aspect, the RCC to be treated is a clear cell renal cell carcinoma. In another aspect, the RCC to be treated has invaded the renal sinus. In some aspects, the RCC is a TNM stage III disease. In additional aspects, the RCC is a TNM stage IV disease. In additional aspects, the RCC is found within the intrarenal veins. In further aspects, the RCC has metastasized to the adrenal gland or to a lymph node. In further aspects, the RCC has metastasized to the lung, intra-abdominal lymph nodes, bone, brain, or liver.

In another aspect, the disclosure provides a method of treating renal cell carcinoma in a mammal having previously received an RCC therapeutic agent, the method comprising administering to the mammal an effective amount of an activin-like kinase I (ALK1)-Fc fusion protein. In one aspect, the previously received therapeutic agent is a VEGF receptor tyrosine kinase inhibitor. In a further aspect, the VEGF receptor tyrosine kinase inhibitor is an agent selected from: sunitinib, sorafenib, pazopanib, axitinib, tivozanib and vandetanib. In another aspect, the previously received therapeutic agent is a mammalian target of rapamycin (mTOR)-targeted inhibitor. In a further aspect, the mTOR-targeted inhibitor is an agent selected from: everolimus and temsirolimus. In other aspects, the mTOR-targeted inhibitor is an agent selected from: WYE354, YE132 (Pfizer), PP30 and PP242, AZD8055, OSI-027, Torin1, BEZ235, XL765, GDC-0980, PF-04691502 and PF-05212384. In an additional aspect, the previously received therapeutic agent is a systemic cytokine therapy. In a further aspect, the systemic cytokine therapy is interferon alpha (IFN-α) or interleukin-2 (IL-2). According to one aspect the treated RCC is a clear cell renal cell carcinoma. In another aspect, the treated RCC has invaded the renal sinus. In some aspects, the RCC is a TNM stage III disease. In additional aspects, the RCC is a TNM stage IV disease. In additional aspects, the RCC is found within the intrarenal veins. In further aspects, the RCC has metastasized to the adrenal gland or to a lymph node. In further aspects, the RCC has metastasized to the lung, intra-abdominal lymph nodes, bone, brain, or liver.

In additional aspects, the disclosure provides a method of treating renal cell carcinoma in a mammal having previously received an RCC therapeutic agent, the method comprising administering to the mammal an effective amount of an activin-like kinase I (ALK1)-Fc fusion protein and a VEGF receptor tyrosine kinase inhibitor. In a further embodiment, the VEGF receptor tyrosine kinase inhibitor is an agent selected from: sunitinib, sorafenib, pazopanib, axitinib, tivozanib and vandetanib. In another aspect, the treated RCC has invaded the renal sinus. According to one aspect the RCC is a clear cell renal cell carcinoma. In another aspect, the treated RCC has invaded the renal sinus. In some aspects, the RCC is a TNM stage III disease. In additional aspects, the RCC is a TNM stage IV disease. In additional aspects, the RCC is found within the intrarenal veins. In farther aspects, the RCC has metastasized to the adrenal gland or to a lymph node. In further aspects, the RCC has metastasized to the lung, intra-abdominal lymph nodes, bone, brain, or liver.

In additional aspects, the disclosure provides a method of treating renal cell carcinoma in a mammal having previously received an RCC therapeutic agent, the method comprising administering to the mammal an effective amount of an activin-like kinase I (ALK1)-Fc fusion protein and an antibody that binds a receptor tyrosine kinase. In a further aspect, the antibody binds a receptor tyrosine kinase selected from: VEGF, VEGFR1, VEGFR2, VEGFR3, PDGFRa, PDGFRb, c-KIT, MET FAK, RET, beta FGF, TiE-1, Tie-2 and EGFR. In an additional aspect, the administered antibody is bevacizumab. According to one aspect the RCC is a clear cell renal cell carcinoma. In another aspect, the treated RCC has invaded the renal sinus. In some aspects, the RCC is a TNM stage III disease. In additional aspects, the RCC is a TNM stage IV disease. In additional aspects, the RCC is found within the intrarenal veins. In further aspects, the RCC has metastasized to the adrenal gland or to a lymph node. In further aspects, the RCC has metastasized to the lung, intra-abdominal lymph nodes, bone, brain, or liver.

In additional aspects, the disclosure provides a method of treating renal cell carcinoma in a mammal having previously received an RCC therapeutic agent wherein the method comprises administering to the mammal an effective amount of an activin-like kinase I (ALK1)-Fc fusion protein and an mTOR-targeted inhibitor. In a further aspect, mTOR-targeted inhibitor is an agent selected from: everolimus and temsirolimus. In other aspects, the mTOR inhibitor is an agent selected from: WYE354, YE132 (Pfizer), PP30 and PP242, AZD8055, OSI-027, Torin1, BEZ235, XL765, GDC-0980, PF-04691502 and PF-05212384. According to one aspect the RCC is a clear cell renal cell carcinoma. In another aspect, the treated RCC has invaded the renal sinus. In some aspects, the RCC is a TNM stage III disease. In additional aspects, the RCC is a TNM stage IV disease. In additional aspects the RCC is found within the intrarenal veins. In further aspects, the RCC has metastasized to the adrenal gland or to a lymph node. In further aspects, the RCC has metastasized to the lung, intra-abdominal lymph nodes, bone, brain, or liver.

In additional aspects, the disclosure provides a method of treating renal cell carcinoma in a mammal having previously received an RCC therapeutic agent wherein the method comprises administering to the mammal an effective amount of an activin-like kinase I (ALK1)-Fc fusion protein and an immunostimulatory cytokine. In a further embodiment, the administered immunostimulatory cytokine is IFN-α or IL-2. According to another aspect the treated RCC is a clear cell renal cell carcinoma. In another aspect, the treated RCC has invaded the renal sinus. In some aspects, the RCC is a TNM stage III disease. In additional aspects, the RCC is a TNM stage IV disease. In additional aspects, the RCC is found within the intrarenal veins. In further aspects, the RCC has metastasized to the adrenal gland or to a lymph node. In further aspects, the RCC has metastasized to the lung, intra-abdominal lymph nodes, bone, brain, or liver.

In an additional aspect, the disclosure provides a method of treating RCC in a mammal, which comprises administering to a mammal that has RCC and that has undergone or is preparing to undergo a medical procedure to treat RCC, an effective amount of an activin-like kinase I (ALK1)-Fc fusion protein. In one aspect, the medical procedure is selected from: nephron-sparing surgery, a partial nephrectomy, a complete nephrectomy and thermal ablation. In some aspects the RCC is a clear cell renal cell carcinoma. In additional aspects the RCC has invaded the renal sinus. In some aspects, the RCC is a TNM stage III disease % in additional aspects, the RCC is a TNM stage IV disease. In additional aspects, the RCC is found within the intrarenal veins. In further aspects, the RCC has metastasized to the adrenal gland or to a lymph node. In further aspects, the RCC has metastasized to the lung, intra-abdominal lymph nodes, bone, brain, or liver.

In one aspect, the ALK1-Fc fusion protein administered the mammal that has RCC and that has undergone or is preparing to undergo a medical procedure to treat RCC comprises a polypeptide having an amino acid sequence that is at least 85%, 90%, 95%, 96%, 97%, 98%, 99% or 100% identical to the sequence of amino acids 22-118 or 22-120 of SEQ ID NO:1, and wherein the ALK1-Fc fusion protein binds to an ALK1 ligand selected from GDF5, GDF6, GDF7, BMP9 and BMP10. In an additional aspect, the Fc portion of the ALK1-Fc fusion protein is an Fc portion of a human IgG1 immunoglobulin. In a further aspect, the ALK1-Fc fusion protein comprises the amino acid sequence of SEQ ID NO:3 or SEQ ID NO:14

In a further aspect the disclosure provides a method of treating RCC in a mammal that has undergone or is preparing to undergo a medical procedure to treat RCC, wherein the method comprises administering to the mammal an effective amount of an activin-like kinase I (ALK1)-Fc fusion protein and a VEGF receptor tyrosine kinase inhibitor. According to one aspect, the VEGF receptor tyrosine kinase inhibitor is an agent selected from sunitinib, sorafenib, pazopanib, axitinib, tivozanib and vandetanib.

In another aspect the disclosure provides a method of treating RCC in a mammal that has undergone or is preparing to undergo a medical procedure to treat RCC, wherein the method comprises administering to the mammal an effective amount of an ALK1-Fc fusion protein, a VEGF receptor tyrosine kinase inhibitor and an mTOR-targeted inhibitor. In one aspect, the mTOR-targeted inhibitor is an agent selected from: everolimus and temsirolimus. In another aspect, the mTOR inhibitor is an agent selected from: WYE354, YE132 (Pfizer), PP30 and PP242, AZD8055, OSI-027, Torin1, BEZ235, XL765, GDC-0980, PF-04691502 and PF-05212384.

In another aspect the disclosure provides a method of treating RCC in a mammal that has undergone or is preparing to undergo a medical procedure to treat RCC, wherein the method comprises administering to the mammal an effective amount of an ALK1-Fc fusion protein, a VEGF receptor tyrosine kinase inhibitor and an immunostimulatory cytokine. In one aspect, administered immunostimulary cytokine is IFN-alpha or IL-2.

In certain aspects, the disclosure provides method of treating RCC in a mammal that has undergone or is preparing to undergo a medical procedure to treat RCC wherein the method comprises administering to the mammal an antibody that binds to an ALK1 ligand and inhibits the binding of the ALK1 ligand to ALK1. In some embodiments, the antibody binds to the ALK1 ligand with a K_{D} of less than 5×10⁻⁸ M. In some embodiments, the antibody inhibits angiogenesis stimulated by the ALK1 ligand. In certain aspects, the antibody binds to ALK1 in the extracellular domain, amino acids 22-118 or 22-120 of SEQ ID NO:1 and inhibit the binding of ALK1 to at least one ALK1 ligand selected from the group consisting of: GDF5, GDF6, GDF7, BMP9 and BMP10. Based on the affinity of these ligands for ALK1, an antibody may bind with a K_{D} of less than 5×10⁻⁸ M, and optionally between 5×10⁻⁸ M and 1×10⁻¹⁰ M. An antibody with affinity within this range would be expected to inhibit signaling by one or more of GDF5, GFD6 and GFD7 while having less effect on signaling by BMP9 and BMP10. Such an antibody preferably inhibits angiogenesis stimulated by at least one ALK1 ligand selected from the group consisting of: GDF5, GDF6 and GDF7. While not wishing to be bound to a particular mechanism, it is expected that such antibodies will act by inhibiting ALK1 activity directly, which should be contrasted to the activity of an ALK1-Fc fusion protein, which is expected to inhibit the activity of ALK1 ligands. An anti-ALK1 antibody is not expected to interfere with the ability of GDF5, GDF6, GDF7, BMP9 or BMP 10 to signal through alternative receptor systems, such as the BMPR1a, BMPR1b and BMPR11 complexes. However, an anti-ALK1 antibody is expected to interfere with the ability of low affinity ligands for ALK1 (e.g., TGF-β, which is generally recognized as triggering significant signaling events through ALK1 even though binding is relatively weak) to signal through ALK1, even though an ALK1 ECD may not bind to or inhibit such low affinity ligands. In some embodiments, an bind to the ALK1 polypeptide with a K_{D} of less than 1×10⁻¹⁰ M. An antibody with affinity within this range would be expected to inhibit signaling by BMP9 or BMP10. Such an antibody preferably inhibits binding of BMP9 and BMP10 to ALK1.

In order to form a functional signaling complex, members of the BMP/GDF family, including BMP9, BMP10, GDF5, GDF6 and GDF7, bind to a type I and a type II receptor. The binding sites for these two types of receptors are different. Accordingly, in certain embodiments, an antibody that binds to an ALK1 ligand and inhibits the ligand to ALK1 is an antibody that binds at or near the type I receptor binding site of the ligand.

Notably, based on the data disclosed herein, an antibody that binds relatively poorly to ALK1 may inhibit TGFβ binding to ALK1 while failing to inhibit the tighter binding ligands such as GDF5 or BMP9. The antibodies described herein are preferably recombinant antibodies, meaning an antibody expressed from a nucleic acid that has been constructed using the techniques of molecular biology, such as a humanized antibody or a fully human antibody developed from a single chain antibody. Fv, Fab and single chain antibodies are also included within the term “recombinant antibody.” Antibodies may also be polyclonal or non-recombinant monoclonal antibodies (including human or murine forms, as well as human antibodies obtained from transgenic mice). Antibodies and ALK1-ECD polypeptides can readily be formulated as a pharmaceutical preparation that is substantially pyrogen free. The pharmaceutical preparation can be prepared for systemic delivery (e.g., intravenous, intraarterial or subcutaneous delivery) or local delivery. Antibodies described in Intl. Appl. Publ. No. WO 2007/040912 may be useful in the various methods described herein.

In certain aspects, the disclosure provides methods for treating renal cell carcinoma in a mammal by administering to a mammal an effective amount of an antibody that binds to an ALK1 polypeptide, described herein either generally or specifically. In one aspect, the renal cell carcinoma is a clear cell renal cell carcinoma. In another aspect, the RCC has invaded the renal sinus. In some aspects, the RCC is a TNM stage III disease. In additional aspects, the RCC is a TNM stage IV disease. In additional aspects, the RCC is found within the intrarenal veins. In further aspects, the RCC has metastasized to the adrenal gland or to a lymph node. In further aspects, the RCC has metastasized to the lung, intra-abdominal lymph nodes, bone, brain, or liver.

An antibody useful for this purpose binds to the extracellular domain of ALK1 (e.g., bind to a polypeptide consisting of amino acids 22-118 of SEQ ID NO:1) or another portion of ALK1. In one embodiment, the antibody binds to a polypeptide consisting of amino acids 22-118 of SEQ ID NO:1 and inhibits the binding of at least one ALK1 ligand selected from the group consisting of: GDF5, GDF6, GDF7, BMP9 and BMP10. In another embodiment, the antibody binds to the ALK1 polypeptide with a K_{D} of less than 5×10⁻⁸ M, and optionally between 5×10⁻⁸ M and 1×10⁻¹° M. In an additional embodiment, the antibody inhibits angiogenesis stimulated by at least one ALK1 ligand selected from the group consisting of: GDF5, GDF6 and GDF7. In some embodiments, an antibody that selectively inhibits signaling mediated by GDF5, GDF6 or GDF7 relative to signaling by BMP9 or BMP 10 is used as a selective inhibitor of angiogenesis that occurs tissues where GDF5, GDF6 or GDF7 are localized: primarily bone or joints. In some embodiments, the antibody binds to ALK1 polypeptide with a K_{D} of less than 1×10⁻¹⁰ M. In additional embodiments, the antibody inhibits the binding of ALK1 to an ALK1 ligand, wherein the ALK1 ligand is selected from the group consisting of: BMP9 and BMP10. The anti-ALK1 antibody may be delivered locally or systemically (e.g., intravenously, intraarterially or subcutaneously). In a particular embodiment, the disclosure provides a method for treating advanced renal cell carcinoma of a mammal by administering an anti-ALK1 antibody.

In another particular embodiment, the disclosure provides a method for treating a mammal having renal cell carcinoma by administering an anti-ALK1 antibody and a VEGF receptor tyrosine kinase inhibitor as described herein. In a particular embodiment, the disclosure provides a method for treating a mammal having clear cell renal cell carcinoma by administering an anti-ALK1 antibody and a VEGF receptor tyrosine kinase inhibitor to a mammal having RCC. In one aspect, the RCC is a clear cell renal cell carcinoma. In another aspect, the RCC to be treated has invaded the renal sinus. In some aspects, the RCC is a TNM stage III disease. In additional aspects, the RCC is a TNM stage IV disease. In additional aspects, the RCC is found within the intrarenal veins. In further aspects, the RCC has metastasized to the adrenal gland or to a lymph node. In further aspects, the RCC has metastasized to the lung, intra-abdominal lymph nodes, bone, brain, or liver.

In certain aspects, the disclosure provides compositions containing a VEGF receptor tyrosine kinase inhibitors and antibodies that bind to an ALK1 ligand and inhibit the binding of the ALK1 ligand to ALK1, wherein the ALK1 ligand is selected from the group consisting of BMP9 and BMP10. Notably, as shown herein, a neutralizing anti-BMP9 antibody inhibits angiogenesis in vivo. Additionally, as demonstrated herein, BMP-10 stimulates angiogenesis while an antagonist of BMP-10 inhibits angiogenesis. The antibody may bind to the ALK1 ligand with a K_{D} of less than 1×10⁻¹⁰ M. Such antibodies are preferably recombinant antibodies, and may be formulated as a pharmaceutical preparation that is substantially pyrogen free. The pharmaceutical preparation may be prepared for systemic delivery (e.g., intravenous, intraarterial or subcutaneous delivery) or local delivery.

In certain aspects, the disclosure provides methods for treating renal cell carcinoma in a mammal, the method comprising, administering to the mammal an effective amount of a receptor tyrosine kinase inhibitor (RTKI) and an antibody that binds to an ALK1 ligand and inhibits the binding of the ALK1 ligand to ALK1, wherein the ALK1 ligand is selected from the group consisting of GDF5, GDF6, GDF7, BMP9 and BMP10. The antibody may inhibit angiogenesis stimulated by at least one ALK1 ligand selected from the group consisting of: GDF5, GDF6 and GDF7. In further aspects, the treated renal cell carcinoma has metastasized to a lymph node. In additional aspects, the treated renal cell carcinoma is clear cell renal cell carcinoma.

In certain aspects, the disclosure provides methods for treating renal cell carcinoma in a mammal by administering to a mammal having RCC an effective amount of a VEGF receptor tyrosine kinase inhibitor and an inhibitor of the ALK1 signaling system, including but not limited to, nucleic acids (e.g., antisense or RNAi constructs) that decrease the production of ALK1, GDF5, GDF6, GDF7, BMP9 or BMP10. In another aspect, the RCC to be treated has invaded the renal sinus. In some aspects, the RCC is a TNM stage III disease. In additional aspects, the RCC is a TNM stage IV disease. In additional aspects, the RCC is found within the intrarenal veins. In further aspects, the RCC has metastasized to the adrenal gland or to a lymph node. In further aspects, the RCC has metastasized to the lung, intra-abdominal lymph nodes, bone, brain, or liver. Such inhibitors of ALK1 signaling include but are not limited to, affinity binding reagents such as aptamers, random peptides, and protein scaffolds that can be modified to allow binding to selected targets (examples of such scaffolds include anticalins and FNIII domains). These binding reagents can be used to identify and select affinity binding reagents that disrupt the ALK1 regulatory system, either by disrupting the ALK1-ligand interaction or by inhibiting the signaling that occurs after binding. In one aspect, the RCC treated according to this method is a clear cell renal cell carcinoma. In another aspect, the RCC to be treated has invaded the renal sinus. In some aspects, the RCC is a TNM stage III disease. In additional aspects, the RCC is a TNM stage IV disease. In additional aspects, the RCC is found within the intrarenal veins. In further aspects, the RCC has metastasized to the adrenal gland or to a lymph node. In further aspects, the RCC has metastasized to the lung, intra-abdominal lymph nodes, bone, brain, or liver.

In a further aspect of the disclosure a method of treating renal cell carcinoma in a mammal is provided that comprises administering to a mammal having RCC an effective amount of an antagonist of BMP9 and/or BMP10 and a VEGF receptor tyrosine kinase inhibitor. In some embodiments, the antagonist is an antibody that binds to BMP9 and/or BMP10. The antibody can be a polyclonal, monoclonal, and chimeric or a humanized antibody. The antagonist can be an Fd, Fv, Fab, F(ab′), F(ab)₂, or F(ab′)₂ fragment, single chain Fv (scFv), diabody, triabody, tetrabody, minibody or a peptibody. In some embodiments the antagonist is an aptamer (peptide or nucleic acid). Given the overlapping effects of antagonists of BMP9 and BMP10, as demonstrated herein, the disclosures provides for antagonists of both BMP9 and BMP10, such as antibodies that cross-react and thus antagonize both proteins effectively (e.g., affinity less than 10 nM or less than 1 nM for both BMP9 and BMP10). Another example of an ALK1 antagonist that binds both BMP9 and BMP10 is an ALK1-Fc fusion protein which binds to both BMP9 and BMP10 and inhibits the activities of both ligands. In a further aspect of the invention, the method further comprises administering to the mammal an effective amount of an mTOR-targeted inhibitor. In further aspects, the antagonist inhibits BMP9 and/or BMP10 expression. In some embodiments the antagonist is a nucleic acid that inhibits BMP9 and/or BMP10 expression. For example, in one aspect, the nucleic acid is an antisense or RNAi nucleic acid. In other aspects the antagonist is a protein other than an antibody, that binds to BMP9 and/or BMP10. In one aspect the antagonist is a member of a GDF Trap family. Examples of the GDF Trap family include, but are not limited to, follistatin, FLRG, noggin and gremlin. In some embodiments, the antagonist is a polypeptide that comprises an amino acid sequence selected from a library of amino acid sequences by a method that includes a step that detects amino acid sequences that bind to BMP9 and BMP10.

In certain aspects the disclosure provides a method for treating metastatic renal cell carcinoma in a mammal. For example, such a method may comprise administering to a mammal that has metastatic renal cell carcinoma an effective amount of an RTKI and an agent selected from the group consisting of: an ALK1 ECD protein; an antibody that binds to an ALK1 ligand and inhibits the binding of the ALK1 ligand to ALK1, wherein the ALK1 ligand is selected from the group consisting of GDF5, GDF6, GDF7, BMP9 and BMP10; an antibody that binds to an ALK1 polypeptide consisting of amino acids 22-118 of SEQ ID NO:1 and inhibits the binding of at least one ALK1 ligand selected from the group consisting of: GDF5, GDF6, GDF7, BMP9 and BMP10.

In each instance, an agent described herein may be administered in conjunction with an additional agent that inhibits angiogenesis.

In some embodiments, the invention provides methods for inhibiting angiogenesis in a mammal comprising administering to a mammal in need thereof, an effective amount of an inhibitor of the ALK1 signaling system (e.g., ALK1-Fc). Where it is desirable to inhibit angiogenesis of a tumor, the agent is optionally administered in conjunction with a second agent that has an anti-cancer effect, such as a chemotherapeutic agent or a biologic anti-cancer agent. In further aspects the agent is administered with an MTOR (mammalian target of rapamycin) inhibitor. In some embodiments, the methods of the invention are used to treat and angiogenesis related disease selected from the group consisting of a tumor, a tumor that is resistant to anti-VEGF therapy, a multiple myeloma tumor, and a tumor that has metastasized to the lung, intra-abdominal lymph nodes, bone, brain, or liver.

### BRIEF DESCRIPTION OF THE DRAWINGS

FIG. 1 shows the amino acid sequence for the human Activin Like Kinase 1, ALK1 (SEQ ID NO:1). Single underlining shows the predicted extracellular domain. Double underlining shows the intracellular domain. The signal peptide and the transmembrane domain are not underlined.

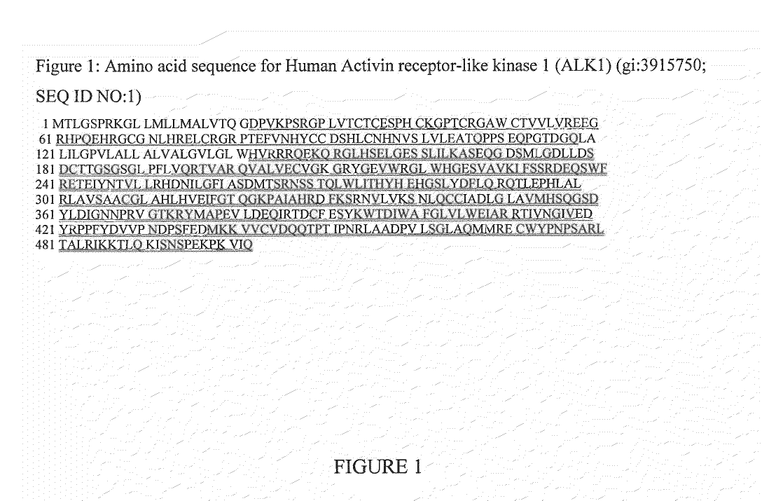

FIG. 2 shows the nucleic acid sequence of a human ALK1 cDNA (SEQ ID NO:2). The coding sequence is underlined. The portion encoding the extracellular domain is double underlined.

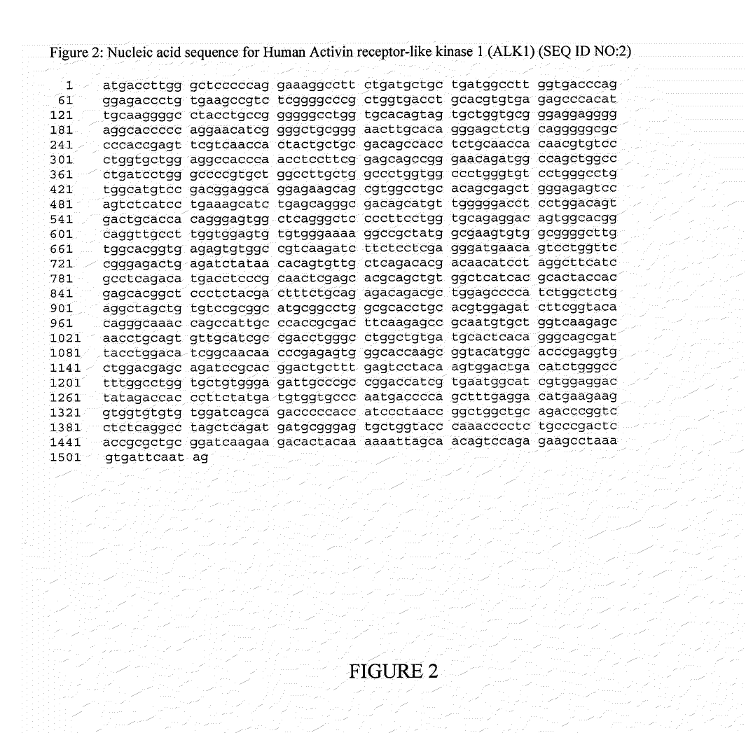

FIGS. 3A and 3B show examples of fusions of the extracellular domain of human ALK1 to an Fc domain (SEQ ID NO:3) and (SEQ ID NO:14). The hALK1-Fc protein includes amino acids 22-120 of the human ALK1 protein, fused at the C-terminus to a linker (underlined) and an IgG1 Fc region.

FIG. 4 shows the nucleic acid sequence for expression of the hALK1-Fc polypeptide of SEQ ID NO:3. The encoded amino acid sequence is also shown. The leader sequence is cleaved such that Asp 22 is the N-terminal amino acid of the secreted protein.

FIG. 5 shows the anti-angiogenic effect of murine ALK1-Fc (“RAP”) and human ALK1-Fc (“ACE”) in an endothelial cell tube forming assay. All concentrations of RAP and ACE reduced the level of tube formation in response to Endothelial Cell Growth Supplement (ECGF) to a greater degree than the positive control, Endostatin.

FIG. 6 shows the angiogenic effect of GDF7 in a chick chorioallantoic membrane (CAM) assay. The GDF7 effect is comparable to that of VEGF.

FIG. 7 shows the anti-angiogenic effect of the human ALK1-Fc fusion in the CAM assay. hALK1-Fc inhibits angiogenesis stimulated by VEGF, FGF and GDF7.

FIG. 8 shows comparative anti-angiogenic effects of murine ALK1-Fc (mALK1-Fc), hALK1-Fc, a commercially available anti-ALK1 monoclonal antibody (Anti-ALK1 mAb) and a commercially available, neutralizing anti-VEGF monoclonal antibody. The anti-angiogenic effect of the ALK1-Fc constructs is comparable to the effects of the anti-VEGF antibody.

FIG. 9 shows the anti-angiogenic effects of hALK1-Fc and the anti-VEGF antibody in vivo. hALK1-Fc and anti-VEGF had comparable effects on angiogenesis in the eye as measured by the mouse corneal micropocket assay.

FIG. 10 shows the effects of mALK1-Fc in the murine collagen-induced arthritis (CIA) model of rheumatoid arthritis. The graph shows mean group arthritic scores determined during the 42 day observation period in the collagen-induced male DBA/1 arthritic mice. RAP-041 is mALK1-Fc. Avastin™ is the anti-VEGF antibody bevacizumab.

FIG. 11 shows resolution of hALK1-Fc (SEQ ID NO: 3) and an hALK1-Fc fusion protein from R&D Systems (Minneapolis, Minn.) by Superose 12 10/300 GL Size Exclusion column (Amersham Biosciences, Piscataway, N.J.). The R&D Systems material contains approximately 13% aggregated protein, as shown by the peaks on the left hand side of the graph, as well as some lower molecular weight species. The material of SEQ ID NO:3 is greater than 99% composed of dimers of the appropriate molecular size.

FIG. 12 shows fluorescent signal from luciferase-expressing Lewis lung cancer (LL/2-luc) cells in mice treated with PBS (circles) and mALK1-Fc (squares). Tumor cells were injected into the tail vein and treatment (PBS or 10 mg/kg mALK1-Fc IP, twice weekly) was initiated on the day of cell administration. PBS-treated mice were sacrificed on day 22 as being moribund. The treatment and control groups each consisted of seven animals (n=7).

FIG. 13 shows the effect of recombinant human BMP9 (“rhB9”) and a commercially available anti-BMP9 monoclonal antibody (“mabB9”) on VEGF-mediated angiogenesis in the CAM assay. Intriguingly, both BMP9 and anti-BMP9 treatment inhibit VEGF-mediated angiogenesis.

FIG. 14 shows the effects of mALK1-Fc on an orthotopic xenograft model using the MDA-MB-231 cell line, a cell line derived from ER− breast cancer cells. At a dose of 30 mg/kg, the mALK1-Fc has a significant growth-delaying effect on the xenograft tumor.

FIG. 15 shows the effects of hALK1-Fc on an orthotopic xenograft model using the MCF7 cell line, a cell line derived from estrogen receptor positive (ER+) breast cancer cells. At a dose of 10 or 30 mg/kg, the hALK1-Fc has a significant growth-delaying effect on the xenograft tumor.

FIG. 16 shows the ability of hALK1-Fc to inhibit by more than 80% the transcriptional reporter activity induced by BMP 10 in a cell-based assay.

FIG. 17 shows an alignment of the mature portions of the human BMP9 (SEQ ID NO:12) and BMP10 (SEQ ID NO:13) proteins. Regions of identity are shown with asterisks.

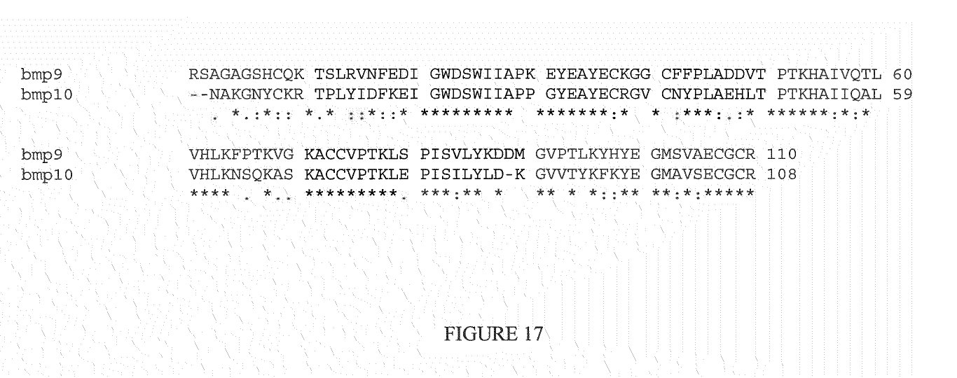

FIG. 18 shows the ability of hALK1-Fc to enhance tumor growth inhibition by sunitinib in a 786-O human RCC xenograft model. hALK1-Fc additional trended toward inhibiting tumor growth as a single agent.

FIG. 19 shows the ability of hALK1-Fc to inhibit tumor growth as a single agent in an A498 human RCC xenograft model.

FIG. 20 shows the ability of hALK1-Fc to enhance tumor growth inhibition by sunitinib in an A498 human RCC xenograft model.

### DETAILED DESCRIPTION

1. Overview

Renal Cell Carcinoma

The World Health Organism lists over 50 different types of kidney cancer. Renal cell carcinoma (RCC) is the most common type of kidney cancer in adults and arises when cancer cells form in the lining of tubules in the kidney. RCC is characterized by a lack of early warning signs, diverse clinical manifestations and resistance to chemotherapy and radiation. Most RCC tumors present in patients between 50 and 70 years of age and the incidence of the disease is two to three times higher in men. Certain genetic conditions are associated with an increased incidence of RCC including von Hippel-Lindau (VHL) syndrome, hereditary papillary renal carcinoma, familial renal oncocytoma associated with Birt-Hogg-Dube syndrome and hereditary renal carcinoma. 30% of patients present at advance stages of RCC, having either metastatic or unresectable disease, and the 2-year overall survival of this cohort is <10%. Reeves et al., Cancer Chemotherapy and Pharmacology 2009; 64(1):11-25.

Five major subtypes of RCC are currently recognized including clear cell, the most common RCC subtype, papillary (type I and type II), chromophobe, collecting duct, and unclassified RCC. Moreover, anatomical criteria has been traditionally used to differentiate the distinct stages of RCC. The tumor, nodes and metastases (TMN) classification system is based on the primary size of the tumor, the degree of tumor spread to the lymph nodes, and the presence of metastasis to differentiate the stages of RCC. Tumor stage is the most important factor predictive of survival in RCC. Koul et al., Am. J. Cancer Research 2011; 1(2); 240-254. More than 50% of patients with early stage RCC are cured. Under certain circumstances radical nephrectomy is also indicated to treat locally advanced RCC and metastatic RCC. 23% of patients with clinically localized disease develop metastatic disease after nephrectomy. Koul et al., Am. J. Cancer Research 2011; 1(2):240-254, However, the outcome is poor for TNM stage III and stage IV diseases, which are characterized by for example, the presence of the tumor in the major veins or adrenal gland, or lymph node involvement (stage III) and the presence of disease outside of the kidney (IV).

Clear cell renal cell carcinoma typically arises within the renal cortex from epithelial cells of the proximal convoluted tubules of the nephron and tends to spread through vascular invasion, with malignant cells found within intrarenal veins in 18-29% of organ-confined tumors. Delahunt et al., Clin. Lab. Med. 2005; 25(2):231-46; and Bonsib et al., Mod. Pathol. 2006; 19(5):746-53. Extensive pathologic examinations of 120 clear cell renal cell carcinomas have indicated renal sinus invasion in approximately half of the tumors studied. RCC most commonly metastasizes to the lung (33-72%), intra-abdominal lymph nodes (3-35%), bone (21-25%), brain (7-13%) and liver (5-10%). See, e.g., Klatte et al., Urol. Oncol. 2008; 26(6):604-9.

Small tumors localized to or within the kidney are frequently removed by partial nephrectomy (also known as “nephron-sparing surgery”). Additional surgical procedures for localized tumors include tissue ablation treatments (e.g., cryosurgery and radiofrequency ablation (RFA). In those instances where the cancer is advanced in size and/or distribution within or beyond the kidney, surgical intervention typically involves a complete nephrectomy (i.e., the removal of the entire kidney with or without the nearby adrenal gland and the fatty tissue around the kidney). This surgery is the traditional standard intervention for kidney cancer. Under certain circumstances radical nephrectomy is also indicated to treat locally advanced RCC and metastatic RCC. 23% of patients with clinically localized disease develop metastatic disease after nephrectomy. Koul et al., Am J Cancer Research 2011; 1(2):240-254.

Immunotherapy with immunostimulatory cytokines such as interleukin-2 (IL-2) and interferon-α (IFN-α) is the mainstay systematic therapy for RCC. High-dose intravenous IL-2 has been reported to produce a 15-20% response rate, 6-8% complete remission rate, and approximately 5% cure rate. Koul et al., Am J Cancer Research 2011; 1(2):240-254. However, the regime is fairly toxic. IFN-α produced a more modest survival benefit but has a more favorable toxicity profile

ALK1

ALK1 is a type I cell-surface receptor for the TGF-β superfamily of ligands and is also known as ACVRL1 and ACVRLK1. ALK1 has been implicated as a receptor for TGF-β1, TGF-β3 and BMP-9 (Marchuk et al., 2003; Hum. Mol. Genet. 12:R97-R112 and Brown et al., 2005; J. Biol. Chem. 280(26):25111-8).

In mice, loss-of-function mutations in ALK1 lead to a variety of abnormalities in the developing vasculature (Oh et al., 2000; Proc. Natl. Acad. Sci. USA 97:2626-31 and Urness et al., 2000; Nat. Genet. 26:328-31).

In humans, loss-of-function mutations in ALK1 are associated with hereditary hemorrhagic telangiectasia (HHT, or Osler-Rendu-Weber syndrome), in which patients develop arteriovenous malformations that create direct flow (communication) from an artery to a vein (arteriovenous shunt), without an intervening capillary bed. Typical symptoms of patients with HHT include recurrent epistaxis, gastrointestinal hemorrhage, cutaneous and mucocutaneous telangiectases, and arteriovenous malformations (AVM) in the pulmonary, cerebral, or hepatic vasculature.

Recent publications from David et al., (Blood 2007; 109(5):1953-61) and Scharpfenecker et al., (J. Cell Sci. 2007 120(6):964-72) concluded that BMP9 and BMP10 activate ALK1 in endothelial cells, and that the consequence of this activation is to inhibit endothelial cell proliferation and migration. These proposed effects of ALK1 activation are directly opposed to those of pro-angiogenic factors such as VEGF. Thus, these publications conclude that BMP9 and BMP10 are themselves anti-angiogenic factors, and further, that ALK1 activation has an anti-angiogenic effect. By contrast, the present disclosure demonstrates that antagonists, rather than agonists, of BMP9 and BMP10 have anti-angiogenic effects.

The disclosure relates to the discovery that polypeptides comprising a portion of the extracellular domain of ALK1 (“ALK1 ECD polypeptides”) can inhibit RCC cancer growth in vivo. More particularly, as discussed below, the disclosure describes the use of ALK1 ECD antagonists to demonstrate the involvement of ALK1 in influencing both VEGF-independent angiogenesis and angiogenesis that is mediated by multiple angiogenic factors, including VEGF, FGF and PDGF. The disclosure also relates to the surprising discovery that ALK1 ECD antagonists, such as ALK1-Fc are able to inhibit RCC tumor growth in a human RCC xenograft model in vivo and also to dramatically improve tumor inhibiting activity of the sunitinib in human RCC xenograft models.

The disclosure additionally relates to the discovery that polypeptides comprising a portion of the extracellular domain of ALK1 (“ALK1 ECD polypeptides”) may be used to inhibit angiogenesis in vivo, including both VEGF-independent angiogenesis and angiogenesis that is mediated by multiple angiogenic factors, including VEGF, FGF and PDGF.

The disclosure also relates to the discovery that polypeptides comprising a portion of the extracellular domain of ALK1 (“ALK1 ECD polypeptides”) may be used to inhibit angiogenesis in vivo, including VEGF-independent angiogenesis and angiogenesis that is mediated by multiple angiogenic factors, including VEGF, FGF and PDGF. In part, the disclosure provides the identity of physiological, high affinity ligands for ALK1 and demonstrates that ALK1 ECD polypeptides inhibit angiogenesis.

In part, the disclosure provides the identity of physiological, high affinity ligands for ALK1 and demonstrates that ALK1 ECD polypeptides inhibit angiogenesis. The data presented herein demonstrate that an ALK1 ECD polypeptide can exert an anti-angiogenic effect even in situations where the ALK1 ECD polypeptide does not exhibit meaningful binding to TGF-β1. Moreover, ALK1 ECD polypeptides inhibit angiogenesis that is stimulated by many different pro-angiogenic factors, including VEGF, FGF, and GDF7. Thus, the disclosure provides a description of an ALK1 regulatory system, in which ALK1 is a receptor for the GDF5 group of ligands, which includes GDF6 and GDF7, and also for the BMP9 group of ligands, which includes BMP10, with different affinities for the two groups of ligands. Further, the disclosure demonstrates that signaling mediated by ALK1 and the ligands described above is pro-angiogenic in vivo, and that inhibition of this regulatory system has a potent anti-angiogenic effect in vivo.

Thus, in certain aspects, the disclosure provides antagonists of the ALK1 regulatory system, including antagonists of the ALK1 receptor or one or more of the ALK1 ligands, for use in inhibiting angiogenesis, including both VEGF-dependent angiogenesis and VEGF-independent angiogenesis. However, it should be noted that antibodies directed to ALK1 itself are expected to have different effects from an ALK1 ECD polypeptide. A pan-neutralizing antibody against ALK1 (one that inhibits the binding of all strong and weak ligands) would be expected to inhibit the signaling of such ligands through ALK1 but would not be expected to inhibit the ability of such ligands to signal through other receptors (e.g., BMPR1a, BMPR1b, BMPR11 in the case of GDF5-7 and BMP9-10 and TBRI and TBRII in the case of TGFβ). On the other hand, an ALK1 ECD polypeptide would be expected to inhibit all of the ligands that it binds to tightly, including, for example, a construct such as that shown in the Examples, GDF5-7 and BMP9-10, but would not affect ligands that it binds to weakly, such as TGF-β. So, while a pan-neutralizing antibody against ALK1 would block BMP9 and TGF-β signaling through ALK1 the antibody would not block BMP9 and TGF-β signaling through another receptor, and while an ALK1 ECD polypeptide may inhibit BMP9 signaling through all receptors (even receptors other than ALK1) it would not be expected to inhibit TGF-β signaling through any receptor, even ALK1.

The terms used in this specification generally have their ordinary meanings in the art, within the context of this disclosure and in the specific context where each term is used. Certain terms are discussed in the specification, to provide additional guidance to the practitioner in describing the compositions and methods disclosed herein and how to make and use them. The scope or meaning of any use of a term will be apparent from the specific context in which the term is used.

2. Soluble ALK1 Polypeptides

Naturally occurring ALK1 proteins are transmembrane proteins, with a portion of the protein positioned outside the cell (the extracellular portion) and a portion of the protein positioned inside the cell (the intracellular portion). Aspects of the present disclosure encompass polypeptides comprising a portion of the extracellular domain of ALK1.

In certain embodiments, the disclosure provides “ALK1 ECD polypeptides”. The term “ALK1 ECD polypeptide” is intended to refer to a polypeptide consisting of or comprising an amino acid sequence of an extracellular domain of a naturally occurring ALK1 polypeptide, either including or excluding any signal sequence and sequence N-terminal to the signal sequence, or an amino acid sequence that is at least 33 percent identical to an extracellular domain of a naturally occurring ALK1 polypeptide, and optionally at least 60%, at least 70%, at least 80%, at least 85%, at least 90%, at least 95%, at least 96%, at least 97%, at least 98%, at least 99% or 100% identical to the sequence of an extracellular domain of a naturally occurring ALK1 polypeptide, as exemplified by the cysteine knot region of amino acids 34-95 of SEQ ID NO:1 or the cysteine knot plus additional amino acids at the N- and C-termini of the extracellular domain, such as amino acids 22-118 or 22-120 of SEQ ID NO. 1.

Likewise, an ALK1 ECD polypeptide may comprise a polypeptide that is encoded by nucleotides 100-285 of SEQ ID NO:2, or silent variants thereof or nucleic acids that hybridize to the complement thereof under stringent hybridization conditions (generally, such conditions are known in the art but may, for example, involve hybridization in 50% v/v formamide, 5×SSC, 2% w/v blocking agent, 0.1% N-lauroylsarcosine, 0.3% SDS at 65° C. overnight and washing in, for example, 5×SSC at about 65° C.). Additionally, an ALK1 ECD polypeptide may comprise a polypeptide that is encoded by nucleotides 64-384 of SEQ ID NO:2, or silent variants thereof or nucleic acids that hybridize to the complement thereof under stringent hybridization conditions (generally, such conditions are known in the art but may, for example, involve hybridization in 50% v/v formamide, 5×SSC, 2% w/v blocking agent, 0.1% N-lauroylsarcosine, 0.3% SDS at 65° C. overnight and washing in, for example, 5×SSC at about 65° C.). The term “ALK1 ECD polypeptide” accordingly encompasses isolated extracellular portions of ALK1 polypeptides, variants thereof (including variants that comprise, for example, no more than 2, 3, 4, 5 or 10 amino acid substitutions, additions or deletions in the sequence corresponding to amino acids 22-118 or 22-120 of SEQ ID NO:1 and including variants that comprise no more than 2, 3, 4, 5, or 10 amino acid substitutions, additions or deletions in the sequence corresponding to amino acids 34-95 of SEQ ID NO:1), fragments thereof and fusion proteins comprising any of the preceding, but in each case preferably any of the foregoing ALK1 ECD polypeptides will retain substantial affinity for one or more of GDF5, GDF6, GDF7 BMP9 or BMP10. The term “ALK1 ECD polypeptide” is explicitly intended to exclude any full-length, naturally occurring ALK1 polypeptide. Generally, an ALK1 ECD polypeptide will be designed to be soluble in aqueous solutions at biologically relevant temperatures, pH levels and osmolarity.

As described above, the disclosure provides ALK1 ECD polypeptides sharing a specified degree of sequence identity or similarity to a naturally occurring ALK1 polypeptide. To determine the percent identity of two amino acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues at corresponding amino acid positions are then compared. When a position in the first sequence is occupied by the same amino acid residue as the corresponding position in the second sequence, then the molecules are identical at that position (as used herein amino acid “identity” is equivalent to amino acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity and similarity between two sequences can be accomplished using a mathematical algorithm. (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

In one embodiment, the percent identity between two amino acid sequences is determined using the Needleman and Wunsch (J. Mol. Biol. 1970; (48):444-453) algorithm which has been incorporated into the GAP program in the GCG software package (available at http://www.gcg.com). In a specific embodiment, the following parameters are used in the GAP program: either a Blosum 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package (Devereux et al., Nucleic Acids Res. 1984; 12(1):387) (available at http://www.gcg.com). Exemplary parameters include using a NWSgapdna.CMP matrix and a gap weight of 40, 50, 60, 70, or 80 and a length weight of 1, 2, 3, 4, 5, or 6. Unless otherwise specified, percent identity between two amino acid sequences is to be determined using the GAP program using a Blosum 62 matrix, a GAP weight of 10 and a length weight of 3, and if such algorithm cannot compute the desired percent identity, a suitable alternative disclosed herein should be selected.

In another embodiment, the percent identity between two amino acid sequences is determined using the algorithm of E. Myers and W. Miller (CABIOS, 1989; 4:11-17) which has been incorporated into the ALIGN program (version 2.0), using a PAM120 weight residue table, a gap length penalty of 12 and a gap penalty of 4.

Another embodiment for determining the best overall alignment between two amino acid sequences can be determined using the FASTDB computer program based on the algorithm of Brutlag et al., (Comp. App. Biosci., 1990; 6:237-245). In a sequence alignment the query and subject sequences are both amino acid sequences. The result of said global sequence alignment is presented in terms of percent identity. In one embodiment, amino acid sequence identity is performed using the FASTDB computer program based on the algorithm of Brutlag et al., (Comp. App. Biosci., 1990; 6:237-245). In a specific embodiment, parameters employed to calculate percent identity and similarity of an amino acid alignment comprise: Matrix=PAM 150, k-tuple=2, Mismatch Penalty=1, Joining Penalty=20, Randomization Group Length=0, Cutoff Score=1, Gap Penalty=5 and Gap Size Penalty-0.05.

In certain embodiments, ALK1 ECD polypeptides comprise an extracellular portion of a naturally occurring ALK1 protein such as a sequence of SEQ ID NO:1, and preferably a ligand binding portion of the ALK1 extracellular domain. In embodiments, a soluble ALK1 ECD polypeptide comprises an amino acid sequence that is at least 60%, 70%, 80%, 85%, 90%, 95%, 96%, 97%, 98%, or 99% identical to an amino acid sequence of amino acids 22-118 or 22-120 of the SEQ ID NO:1. In certain embodiments, a truncated extracellular ALK1 polypeptide comprises at least 30, 40 or 50 consecutive amino acids of an amino acid sequence of an extracellular portion of SEQ ID NO:1.

In preferred embodiments, an ALK1 ECD polypeptide binds to one or more of GDF5, GDF6, GDF7, BMP9 and BMP10. Optionally the ALK1 polypeptide does not show substantial binding to TGF-β1 or TGF-β3. Binding may be assessed using purified proteins in solution or in a surface plasmon resonance system, such as a Biacore™ system. Preferred soluble ALK1 polypeptides will exhibit an anti-angiogenic activity. Bioassays for angiogenesis inhibitory activity include the chick chorioallantoic membrane (CAM) assay, the mouse corneal micropocket assay, or an assay known in the art for measuring the effect of administering isolated or synthesized proteins on implanted tumors. The CAM assay is described by O'Reilly, et al., in “Angiogenic Regulation of Metastatic Growth” Cell, 1994; 79 (2):315-328. Briefly, 3 day old chicken embryos with intact yolks are separated from the egg and placed in a petri dish. After 3 days of incubation, a methylcellulose disc containing the protein to be tested is applied to the CAM of individual embryos. After 48 hours of incubation, the embryos and CAMs are observed to determine whether endothelial growth has been inhibited. The mouse corneal micropocket assay involves implanting a growth factor-containing pellet, along with another pellet containing the suspected endothelial growth inhibitor, in the cornea of a mouse and observing the pattern of capillaries that are elaborated in the cornea. Other assays are described in the Examples.

ALK1 ECD polypeptides may be produced by removing the cytoplasmic tail and the transmembrane region of an ALK1 ECD polypeptide. Alternatively, the transmembrane domain may be inactivated by deletion, or by substitution of the normally hydrophobic amino acid residues which comprise a transmembrane domain with hydrophilic ones. In either case, a substantially hydrophilic hydropathy profile is created which will reduce lipid affinity and improve aqueous solubility. Deletion of the transmembrane domain is preferred over substitution with hydrophilic amino acid residues because it avoids introducing potentially immunogenic epitopes.

ALK1 ECD polypeptides may additionally include any of various leader sequences at the N-terminus. Such a sequence would allow the peptides to be expressed and targeted to the secretion pathway in a eukaryotic system. See, e.g., Ernst et al., U.S. Pat. No. 5,082,783. Alternatively, a native ALK1 signal sequence may be used to effect extrusion from the cell. Possible leader sequences include native, tPa and honeybee mellitin leaders (SEQ ID Nos. 7-9, respectively). Processing of signal peptides may vary depending on the leader sequence chosen, the cell type used and culture conditions, among other variables, and therefore actual N-terminal start sites for mature ALK1 ECD polypeptides, including that of SEQ ID NO:5, may shift by 1-5 amino acids in either the N-terminal or C-terminal direction.

In certain embodiments, the present disclosure contemplates specific mutations of the ALK1 polypeptides so as to alter the glycosylation of the polypeptide. Such mutations may be selected so as to introduce or eliminate one or more glycosylation sites, such as O-linked or N-linked glycosylation sites. Asparagine-linked glycosylation recognition sites generally comprise a tripeptide sequence, asparagine-X-threonine (or asparagines-X-serine) (where “X” is any amino acid) which is specifically recognized by appropriate cellular glycosylation enzymes. The alteration may also be made by the addition of, or substitution by, one or more serine or threonine residues to the sequence of the wild-type ALK1 polypeptide (for O-linked glycosylation sites). A variety of amino acid substitutions or deletions at one or both of the first or third amino acid positions of a glycosylation recognition site (and/or amino acid deletion at the second position) results in non-glycosylation at the modified tripeptide sequence. Another means of increasing the number of carbohydrate moieties on an ALK1 polypeptide is by chemical or enzymatic coupling of glycosides to the ALK1 polypeptide. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. These methods are described in WO 87/05330 published Sep. 11, 1987, and in Aplin and Wriston, (1981) CRC Crit. Rev. Biochem., pp. 259-306, incorporated by reference herein. Removal of one or more carbohydrate moieties present on an ALK1 polypeptide may be accomplished chemically and/or enzymatically. Chemical deglycosylation may involve, for example, exposure of the ALK1 polypeptide to the compound trifluoromethanesulfonic acid, or an equivalent compound. This treatment results in the cleavage of most or all sugars except the linking sugar (N-acetylglucosamine or N-acetylgalactosamine), while leaving the amino acid sequence intact. Chemical deglycosylation is further described by Hakimuddin et al., (1987) Arch. Biochem. Biophys. 259:52 and by Edge et al., Anal. Biochem. 1981; 118:131. Enzymatic cleavage of carbohydrate moieties on ALK1 polypeptides can be achieved by the use of a variety of endo- and exo-glycosidases as described by Thotakura et al., (1987) Meth. Enzymol. 138:350. The sequence of an ALK1 polypeptide may be adjusted, as appropriate, depending on the type of expression system used, as mammalian, yeast, insect and plant cells may all introduce differing glycosylation patterns that can be affected by the amino acid sequence of the peptide. In general, ALK1 proteins for use in humans will be expressed in a mammalian cell line that provides proper glycosylation, such as HEK293 or CHO cell lines, although other mammalian expression cell lines, yeast cell lines with engineered glycosylation enzymes and insect cells are expected to be useful as well.

This disclosure further contemplates a method of generating mutants, particularly sets of combinatorial mutants of an ALK1 polypeptide, as well as truncation mutants; pools of combinatorial mutants are especially useful for identifying functional variant sequences. The purpose of screening such combinatorial libraries may be to generate, for example, ALK1 polypeptide variants which can act as either agonists or antagonist, or alternatively, which possess novel activities altogether. A variety of screening assays are provided below, and such assays may be used to evaluate variants. For example, an ALK1 polypeptide variant may be screened for ability to bind to an ALK1 ligand, to prevent binding of an ALK1 ligand to an ALK1 polypeptide or to interfere with signaling caused by an ALK1 ligand. The activity of an ALK1 polypeptide or its variants may also be tested in a cell-based or in vivo assay, particularly any of the assays disclosed in the Examples.

Combinatorially-derived variants can be generated which have a selective or generally increased potency relative to an ALK1 ECD polypeptide comprising an extracellular domain of a naturally occurring ALK1 polypeptide. Likewise, mutagenesis can give rise to variants which have serum half-lives dramatically different than the corresponding wild-type ALK1 ECD polypeptide. For example, the altered protein can be rendered either more stable or less stable to proteolytic degradation or other processes which result in destruction of, or otherwise elimination or inactivation of a native ALK1 ECD polypeptide. Such variants, and the genes which encode them, can be utilized to alter ALK1 ECD polypeptide levels by modulating the half-life of the ALK1 polypeptides. For instance, a short half-life can give rise to more transient biological effects and can allow tighter control of recombinant ALK1 ECD polypeptide levels within the patient. In an Fc fusion protein, mutations may be made in the linker (if any) and/or the Fc portion to alter the half-life of the protein.

A combinatorial library may be produced by way of a degenerate library of genes encoding a library of polypeptides which each include at least a portion of potential ALK1 polypeptide sequences. For instance, a mixture of synthetic oligonucleotides can be enzymatically ligated into gene sequences such that the degenerate set of potential ALK1 polypeptide nucleotide sequences are expressible as individual polypeptides, or alternatively, as a set of larger fusion proteins (e.g., for phage display).

There are many ways by which the library of potential ALK1 ECD variants can be generated from a degenerate oligonucleotide sequence. Chemical synthesis of a degenerate gene sequence can be carried out in an automatic DNA synthesizer, and the synthetic genes then be ligated into an appropriate vector for expression. The synthesis of degenerate oligonucleotides is well known in the art (see for example, Narang, SA Tetrahedron 1983; 39:3; Itakura et al., Recombinant DNA, Proc. 3rd Cleveland Sympos. Macromolecules, ed. AG Walton, Amsterdam: Elsevier pp 273-289; Itakura et al., (1984) Annu. Rev. Biochem. 1981; 53:323; Itakura et al., (1984) Science 1984; 198:1056; Ike et al., Nucleic Acid Res. 1983:1983; 11:477). Such techniques have been employed in the directed evolution of other proteins (see, for example, Scott et al., Science 1990; 249:386-390; Roberts et al., (1992) PNAS USA 89:2429-2433; Devlin et al.,; Science 1990; 249: 404-406; Cwirla et al., (1990) PNAS USA 87: 6378-6382; as well as U.S. Pat. Nos. 5,223,409, 5,198,346, and 5,096,815).

Alternatively, other forms of mutagenesis can be utilized to generate a combinatorial library. For example, ALK1 polypeptide variants can be generated and isolated from a library by screening using, for example, alanine scanning mutagenesis and the like (Ruf et al., Biochemistry 1994; 33:1565-1572; Wang et al., J. Biol. Chem. 1994; 269:3095-3099; Balint et al., Gene 1993; 137:109-118; Grodberg et al., (1993) Eur. J. Biochem. 218:597-601; Nagashima et al., J. Biol. Chem. 1993; 268:2888-2892; Lowman et al., Biochemistry 1991; 30:10832-10838; and Cunningham et al., (1989) Science 244:1081-1085), by linker scanning mutagenesis (Gustin et al., Virology 1993; 193:653-660; Brown et al., Mol. Cell. Biol. 1992; 12:2644-2652; McKnight et al., Science 1982; 232:316); by saturation mutagenesis (Meyers et al., Science 1986; 232:613); by PCR mutagenesis (Leung et al., Method Cell Mol. Biol., 1989; 1:11-19); or by random mutagenesis, including chemical mutagenesis, etc. (Miller et al., (1992) A Short Course in Bacterial Genetics, CSHL Press, Cold Spring Harbor, N.Y.; and Greener et al., Strategies in Mol Biol 1994; 7:32-34). Linker scanning mutagenesis, particularly in a combinatorial setting, is an attractive method for identifying truncated (bioactive) forms of ALK1 polypeptides.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of ALK1 polypeptides. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Preferred assays include ALK1 ligand binding assays and ligand-mediated cell signaling assays.

In certain embodiments, the ALK1 ECD polypeptides may further comprise post-translational modifications in addition to any that are naturally present in the ALK1 polypeptides. Such modifications include, but are not limited to, acetylation, carboxylation, glycosylation, phosphorylation, lipidation, and acylation. As a result, the modified ALK1 ECD polypeptides may contain non-amino acid elements, such as polyethylene glycols, lipids, poly- or mono-saccharide, and phosphates. Effects of such non-amino acid elements on the functionality of an ALK1 ECD polypeptide may be tested as described herein for other ALK1 ECD polypeptide variants. When an ALK1 ECD polypeptide is produced in cells by cleaving a nascent form of the ALK1 polypeptide, post-translational processing may also be important for correct folding and/or function of the protein. Different cells (such as CHO, HeLa, MDCK, 293, W138, NIH-3T3 or HEK293) have specific cellular machinery and characteristic mechanisms for such post-translational activities and may be chosen to ensure the correct modification and processing of the ALK1 polypeptides.

In certain aspects, functional variants or modified forms of the ALK1 ECD polypeptides include fusion proteins having at least a portion of the ALK1 ECD polypeptides and one or more fusion domains. Well known examples of such fusion domains include, but are not limited to, polyhistidine, Glu-Glu, glutathione S transferase (GST), thioredoxin, protein A, protein G, an immunoglobulin heavy chain constant region (Fc), maltose binding protein (MBP), or human serum albumin. A fusion domain may be selected so as to confer a desired property. For example, some fusion domains are particularly useful for isolation of the fusion proteins by affinity chromatography. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QIAexpress™ system (Qiagen) useful with (HIS₆) fusion partners.

As another example, a fusion domain may be selected so as to facilitate detection of the ALK1 ECD polypeptides. Examples of such detection domains include the various fluorescent proteins (e.g., GFP) as well as “epitope tags,” which are usually short peptide sequences for which a specific antibody is available. Well known epitope tags for which specific monoclonal antibodies are readily available include FLAG, influenza virus haemagglutinin (HA), and c-myc tags. In some cases, the fusion domains have a protease cleavage site, such as for Factor Xa or Thrombin, which allows the relevant protease to partially digest the fusion proteins and thereby liberate the recombinant proteins therefrom. The liberated proteins can then be isolated from the fusion domain by subsequent chromatographic separation. In certain preferred embodiments, an ALK1 ECD polypeptide is fused with a domain that stabilizes the ALK1 polypeptide in vivo (a “stabilizer” domain). By “stabilizing” is meant anything that increases serum half life, regardless of whether this is because of decreased destruction, decreased clearance by the kidney, or other pharmacokinetic effect. Fusions with the Fc portion of an immunoglobulin are known to confer desirable pharmacokinetic properties on a wide range of proteins. Likewise, fusions to human serum albumin can confer desirable properties. Other types of fusion domains that may be selected include multimerizing (e.g., dimerizing, tetramerizing) domains and functional domains.

As a specific example, the disclosure provides a fusion protein comprising a soluble extracellular domain of ALK1 fused to an Fc domain (e.g., SEQ ID NO: 6).

THTCPPCPAPELLGGPSVFLFPPKPKDTLMISRTPEVTCVVVD(A)VSHEDPEVKFNWYVDGVEV
HNAKTKPREEQYNSTYRVVSVLTVLHQDWLNGKEYKCK(A)VSNKALPVPIEKTISKAKGQPREP
QVYTLPPSREEMTKNQVSLTCLVKGFYPSDIAVEWESNGQPENNYKTTPPVLDSDGPFFLYSKLT
VDKSRWQQGNVFSCSVMHEALHN(A)HYTQKSLSLSPGK*

Optionally, the Fc domain has one or more mutations at residues such as Asp-265, lysine 322, and Asn-434. In certain cases, the mutant Fc domain having one or more of these mutations (e.g., Asp-265 mutation) has reduced ability of binding to the Fcγ receptor relative to a wildtype Fc domain. In other cases, the mutant Fc domain having one or more of these mutations (e.g., Asn-434 mutation) has increased ability of binding to the MHC class I-related Fc-receptor (FcRN) relative to a wildtype Fc domain.

It is understood that different elements of the fusion proteins may be arranged in any manner that is consistent with the desired functionality. For example, an ALK1 ECD polypeptide may be placed C-terminal to a heterologous domain, or, alternatively, a heterologous domain may be placed C-terminal to an ALK1 ECD polypeptide. The ALK1 ECD polypeptide domain and the heterologous domain need not be adjacent in a fusion protein, and additional domains or amino acid sequences may be included C- or N-terminal to either domain or between the domains.

As used herein, the term, “immunoglobulin Fc region” or simply “Fc” is understood to mean the carboxyl-terminal portion of an immunoglobulin chain constant region, preferably an immunoglobulin heavy chain constant region, or a portion thereof. For example, an immunoglobulin Fc region may comprise 1) a CH1 domain, a CH2 domain, and a CH3 domain, 2) a CH1 domain and a CH2 domain, 3) a CH1 domain and a CH3 domain, 4) a CH2 domain and a CH3 domain, or 5) a combination of two or more domains and an immunoglobulin hinge region. In a preferred embodiment the immunoglobulin Fc region comprises at least an immunoglobulin hinge region a CH2 domain and a CH3 domain, and preferably lacks the CH1 domain.

In one embodiment, the class of immunoglobulin from which the heavy chain constant region is derived is IgG (Igγ) (γ subclasses 1, 2, 3, or 4). Other classes of immunoglobulin, IgA (Igα), IgD (Igδ), IgE (Igε) and IgM (Igμ), may be used. The choice of appropriate immunoglobulin heavy chain constant region is discussed in detail in U.S. Pat. Nos. 5,541,087, and 5,726,044. The choice of particular immunoglobulin heavy chain constant region sequences from certain immunoglobulin classes and subclasses to achieve a particular result is considered to be within the level of skill in the art. The portion of the DNA construct encoding the immunoglobulin Fc region preferably comprises at least a portion of a hinge domain, and preferably at least a portion of a CH₃ domain of Fc gamma or the homologous domains in any of IgA, IgD, IgE, or IgM.

Furthermore, it is contemplated that substitution or deletion of amino acids within the immunoglobulin heavy chain constant regions may be useful in the practice of the methods and compositions disclosed herein. One example would be to introduce amino acid substitutions in the upper CH2 region to create an Fc variant with reduced affinity for Fc receptors (Cole et al., (1997) J. Immunol. 159:3613).

In certain embodiments, the present disclosure makes available isolated and/or purified forms of the ALK1 ECD polypeptides, which are isolated from, or otherwise substantially free of (e.g., at least 80%, 90%, 95%, 96%, 97%, 98%, or 99% free of), other proteins and/or other ALK1 ECD polypeptide species. ALK1 ECD polypeptides will generally be produced by expression from recombinant nucleic acids.

In certain embodiments, the disclosure includes nucleic acids encoding soluble ALK1 polypeptides comprising the coding sequence for an extracellular portion of an ALK1 proteins. In further embodiments, this disclosure also pertains to a host cell comprising such nucleic acids. The host cell may be any prokaryotic or eukaryotic cell. For example, a polypeptide of the present disclosure may be expressed in bacterial cells such as E. coli, insect cells (e.g., using a baculovirus expression system), yeast, or mammalian cells. Other suitable host cells are known to those skilled in the art. Accordingly, some embodiments of the present disclosure further pertain to methods of producing the ALK1 ECD polypeptides. Ad demonstrated herein, an ALK1-Fc fusion protein set forth in SEQ ID NO:14 and expressed in CHO cells has potent anti-angiogenic activity.

3. Nucleic Acids Encoding ALK1 Polypeptides

In certain aspects, the disclosure provides isolated and/or recombinant nucleic acids encoding any of the ALK1 polypeptides (e.g., ALK1 ECD polypeptides), including fragments, functional variants and fusion proteins disclosed herein. For example, SEQ ID NO: 2 encodes the naturally occurring human ALK1 precursor polypeptide, while SEQ ID NO: 4 encodes the precursor of an ALK1 extracellular domain fused to an IgG1 Fc domain. The subject nucleic acids may be single-stranded or double stranded. Such nucleic acids may be DNA or RNA molecules. These nucleic acids may be used, for example, in methods for making ALK1 polypeptides or as direct therapeutic agents (e.g., in an antisense, RNAi or gene therapy approach).

In certain aspects, the subject nucleic acids encoding ALK1 polypeptides are further understood to include nucleic acids that are variants of SEQ ID NO: 2 or 4. Variant nucleotide sequences include sequences that differ by one or more nucleotide substitutions, additions or deletions, such as allelic variants.

In certain embodiments, the disclosure provides isolated or recombinant nucleic acid sequences that are at least 80%, 85%, 90%, 95%, 96%, 97%, 98%, 99% or 100% identical to SEQ ID NO: 2 or 4. One of ordinary skill in the art will appreciate that nucleic acid sequences complementary to SEQ ID NO: 2 or 4, and variants of SEQ ID NO: 2 or 4 are also within the scope of this disclosure. In further embodiments, the nucleic acid sequences of the disclosure can be isolated, recombinant, and/or fused with a heterologous nucleotide sequence, or in a DNA library.

In other embodiments, nucleic acids of the disclosure also include nucleotide sequences that hybridize under highly stringent conditions to the nucleotide sequence designated in SEQ ID NO: 2 or 4, complement sequence of SEQ ID NO: 2 or 4, or fragments thereof. As discussed above, one of ordinary skill in the art will understand readily that appropriate stringency conditions which promote DNA hybridization can be varied. One of ordinary skill in the art will understand readily that appropriate stringency conditions which promote DNA hybridization can be varied. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6×SSC at room temperature followed by a wash at 2×SSC at room temperature.

Isolated nucleic acids which differ from the nucleic acids as set forth in SEQ ID NOs: 2 or 4 due to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this disclosure.

In certain embodiments, the recombinant nucleic acids of the disclosure may be operably linked to one or more regulatory nucleotide sequences in an expression construct. Regulatory nucleotide sequences will generally be appropriate to the host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In a preferred embodiment, the expression vector contains a selectable marker gene to allow the selection of transformed host cells. Selectable marker genes are well known in the art and will vary with the host cell used.

In certain aspects disclosed herein, the subject nucleic acid is provided in an expression vector comprising a nucleotide sequence encoding an ALK1 ECDpolypeptide and operably linked to at least one regulatory sequence. Regulatory sequences are art-recognized and are selected to direct expression of the ALK1 polypeptide. Accordingly, the term regulatory sequence includes promoters, enhancers, and other expression control elements. Exemplary regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding an ALK1 polypeptide. Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

A recombinant nucleic acid included in the disclosure can be produced by ligating the cloned gene, or a portion thereof, into a vector suitable for expression in either prokaryotic cells, eukaryotic cells (yeast, avian, insect or mammalian), or both. Expression vehicles for production of a recombinant ALK1 polypeptide include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures, see Molecular Cloning A Laboratory Manual, 3rd Ed., ed. by Sambrook, Fritsch and Maniatis (Cold Spring Harbor Laboratory Press, 2001). In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

In a preferred embodiment, a vector will be designed for production of the subject ALK1 polypeptides in CHO cells, such as a Pcmv-Script vector (Stratagene, La Jolla, Calif.), pcDNA4 vectors (Invitrogen, Carlsbad, Calif.) and pCI-neo vectors (Promega, Madison, Wisc.). As will be apparent, the subject gene constructs can be used to cause expression of the subject ALK1 polypeptides in cells propagated in culture, e.g., to produce proteins, including fusion proteins or variant proteins, for purification.

This disclosure also pertains to a host cell transfected with a recombinant gene including a coding sequence (e.g., SEQ ID NO: 2 or 4) for one or more of the subject ALK1 ACD polypeptides. The host cell may be any prokaryotic or eukaryotic cell. For example, an ALK1 polypeptide disclosed herein may be expressed in bacterial cells such as E. coli, insect cells (e.g., using a baculovirus expression system), yeast, or mammalian cells. Other suitable host cells are known to those skilled in the art.

Accordingly, the present disclosure further pertains to methods of producing the subject ALK1 polypeptides, including ALK1 ECD polypeptides. For example, a host cell transfected with an expression vector encoding an ALK1 polypeptide can be cultured under appropriate conditions to allow expression of the ALK1 polypeptide to occur. The ALK1 polypeptide may be secreted and isolated from a mixture of cells and medium containing the ALK1 polypeptide. Alternatively, the ALK1 polypeptide may be retained cytoplasmically or in a membrane fraction and the cells harvested, lysed and the protein isolated. A cell culture includes host cells, media and other byproducts. Suitable media for cell culture are well known in the art.

The subject ALK1 polypeptides can be isolated from cell culture medium, host cells, or both, using techniques known in the art for purifying proteins, including ion-exchange chromatography, gel filtration chromatography, ultrafiltration, electrophoresis, immunoaffinity purification with antibodies specific for particular epitopes of the ALK1 polypeptides and affinity purification with an agent that binds to a domain fused to the ALK1 polypeptide (e.g., a protein A column may be used to purify an ALK1-Fc fusion). In a preferred embodiment, the ALK1 polypeptide is a fusion protein containing a domain which facilitates its purification. In a preferred embodiment, purification is achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

In another embodiment, a fusion gene coding for a purification leader sequence, such as a poly-(His)/enterokinase cleavage site sequence at the N-terminus of the desired portion of the recombinant ALK1 polypeptide, can allow purification of the expressed fusion protein by affinity chromatography using a Ni²⁺ metal resin. The purification leader sequence can then be subsequently removed by treatment with enterokinase to provide the purified ALK1 polypeptide (e.g., see Hochuli et al., (1987) J. Chromatography 411:177; and Janknecht et al., PNAS USA 88:8972).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

Examples of categories of nucleic acid compounds that are antagonists of ALK1, BMP9, BMP10, GDF5, GDF6 or GDF7 include antisense nucleic acids, RNAi constructs and catalytic nucleic acid constructs. A nucleic acid compound may be single or double stranded. A double stranded compound may also include regions of overhang or non-complementarity, where one or the other of the strands is single stranded. A single stranded compound may include regions of self-complementarity, meaning that the compound forms a so-called “hairpin” or “stem-loop” structure, with a region of double helical structure. A nucleic acid compound may comprise a nucleotide sequence that is complementary to a region consisting of no more than 1000, no more than 500, no more than 250, no more than 100 or no more than 50, 35, 30, 25, 22, 20 or 18 nucleotides of the full-length ALK1 nucleic acid sequence or ligand nucleic acid sequence. The region of complementarity will preferably be at least 8 nucleotides, and optionally at least 10 or at least 15 nucleotides, and optionally between 15 and 25 nucleotides. A region of complementarity may fall within an intron, a coding sequence or a noncoding sequence of the target transcript, such as the coding sequence portion. Generally, a nucleic acid compound will have a length of about 8 to about 500 nucleotides or base pairs in length, and optionally the length will be about 14 to about 50 nucleotides. A nucleic acid may be a DNA (particularly for use as an antisense), RNA or RNA:DNA hybrid. Any one strand may include a mixture of DNA and RNA, as well as modified forms that cannot readily be classified as either DNA or RNA. Likewise, a double stranded compound may be DNA:DNA, DNA:RNA or RNA:RNA, and any one strand may also include a mixture of DNA and RNA, as well as modified forms that cannot readily be classified as either DNA or RNA. A nucleic acid compound may include any of a variety of modifications, including one or modifications to the backbone (the sugar-phosphate portion in a natural nucleic acid, including internucleotide linkages) or the base portion (the purine or pyrimidine portion of a natural nucleic acid). An antisense nucleic acid compound will preferably have a length of about 15 to about 30 nucleotides and will often contain one or more modifications to improve characteristics such as stability in the serum, in a cell or in a place where the compound is likely to be delivered, such as the stomach in the case of orally delivered compounds and the lung for inhaled compounds. In the case of an RNAi construct, the strand complementary to the target transcript will generally be RNA or modifications thereof. The other strand may be RNA, DNA or any other variation. The duplex portion of double stranded or single stranded “hairpin” RNAi construct will preferably have a length of 18 to 40 nucleotides in length and optionally about 21 to 23 nucleotides in length, so long as it serves as a Dicer substrate. Catalytic or enzymatic nucleic acids may be ribozymes or DNA enzymes and may also contain modified forms. Nucleic acid compounds may inhibit expression of the target by about 50%, 75%, 90% or more when contacted with cells under physiological conditions and at a concentration where a nonsense or sense control has little or no effect. Preferred concentrations for testing the effect of nucleic acid compounds are 1, 5 and 10 micromolar. Nucleic acid compounds may also be tested for effects on, for example, angiogenesis.

4. Antibodies

Another aspect of the disclosure pertains to an antibody reactive with an extracellular portion of an ALK1 polypeptide, preferably antibodies that are specifically reactive with ALK1 polypeptide. In a preferred embodiment, such antibody may interfere with ALK1 binding to a ligand such as GDF5, GDF6, GDF7 BMP-9 or BMP-10—it will be understood that an antibody against a ligand of ALK1 should bind to the mature, processed form of the relevant protein. The disclosure also provides antibodies that bind to GDF5, GDF6, GDF7, BMP9 and/or BMP10 and inhibit ALK1 binding to such ligands. Preferred antibodies will exhibit an anti-angiogenic activity in a bioassay, such as a CAM assay or corneal micropocket assay (see above). A preferred anti-BMP9 antibody is described in Example 10, below. In certain embodiments, an antibody that inhibits both BMP9 and BMP10 may be desirable; such an antibody may inhibit both ligands in an ALK1 binding assay, in an angiogenesis assay (e.g., HUVEC tube forming assay, CAM assay, Matrigel assay, or other such assays described herein).

The term “antibody” as used herein is intended to include whole antibodies, e.g., of any isotype (IgG, IgA, IgM, IgE, etc), and includes fragments or domains of immunoglobulins which are reactive with a selected antigen. Antibodies can be fragmented using conventional techniques and the fragments screened for utility and/or interaction with a specific epitope of interest. Thus, the term includes segments of proteolytically-cleaved or recombinantly-prepared portions of an antibody molecule that are capable of selectively reacting with a certain protein. Non-limiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The term antibody also includes polyclonal, monoclonal, or other purified preparations of antibodies and recombinant antibodies. The term “recombinant antibody”, means an antibody, or antigen binding domain of an immunoglobulin, expressed from a nucleic acid that has been constructed using the techniques of molecular biology, such as a humanized antibody or a fully human antibody developed from a single chain antibody. Single domain and single chain antibodies are also included within the term “recombinant antibody.”

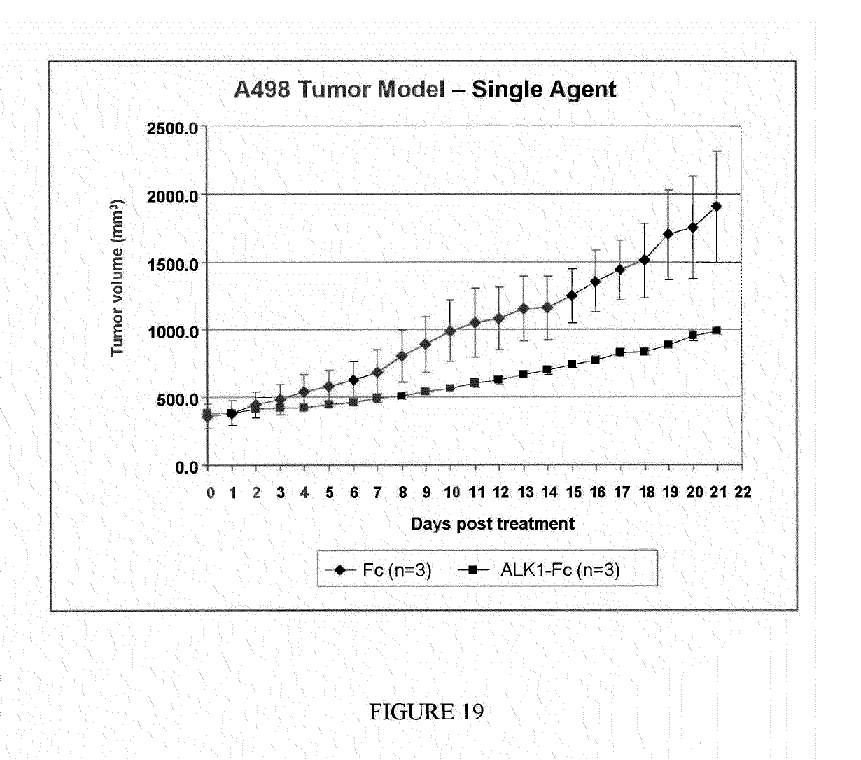

Antibodies may be generated by any of the various methods known in the art, including administration of antigen to an animal, administration of antigen to an animal that carries human immunoglobulin genes, or screening with an antigen against a library of antibodies (often single chain antibodies or antibody domains). Once antigen binding activity is detected, the relevant portions of the protein may be grafted into other antibody frameworks, including full-length IgG frameworks. For example, by using immunogens derived from an ALK1 polypeptide or an ALK1 ligand (e.g., BMP9 or BMP10, or an immunogen common to both BMP9 and BMP10), anti-protein/anti-peptide antisera or monoclonal antibodies can be made by standard protocols (See, for example, Antibodies: A Laboratory Manual ed. by Harlow and Lane (Cold Spring Harbor Press: 1988)). As shown in FIG. 19, BMP9 and BMP10 have considerable amino acid identity, and therefore, each protein may be used as an immunogen to generate antibodies that can cross-react with both BMP9 and BMP10. Fragments of highly similar sequence may also be used as immunogens. A mammal, such as a mouse, a hamster or rabbit can be immunized with an immunogenic form of the peptide (e.g., a ALK1 polypeptide or an antigenic fragment which is capable of eliciting an antibody response, or a fusion protein). Techniques for conferring immunogenicity on a protein or peptide include conjugation to carriers or other techniques well known in the art. An immunogenic portion (preferably an extracellular portion) of an ALK1 polypeptide or an ALK1 ligand such as BMP9 or BMP10 can be administered in the presence of adjuvant. The progress of immunization can be monitored by detection of antibody titers in plasma or serum. Standard ELISA or other immunoassays can be used with the immunogen as antigen to assess the levels of antibodies.

Following immunization of an animal with an antigenic preparation of an ALK1 polypeptide or ligand polypeptide (e.g., BMP9 or BMP10), anti-ALK1 or anti-ligand antisera can be obtained and, if desired, polyclonal antibodies can be isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, Nature, 1975; 256: 495-497), the human B cell hybridoma technique (Kozbar et al., Immunology Today, (1983; 4:72, and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with a mammalian ALK1 polypeptide of the present disclosure or ligands such as BMP9 or BMP10 and monoclonal antibodies isolated from a culture comprising such hybridoma cells. Antibodies with specificity for both BMP9 and BMP10 may be selected from hybridomas that are obtained from animals inoculated with either BMP9 or BMP10 alone.

The term antibody as used herein is intended to include fragments thereof which are also specifically reactive with one of the subject ALK1 polypeptides or ALK1 ligand polypeptides or a combination of target antigens (e.g., BMP9 and BMP10). Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. The antibody of the present disclosure is further intended to include bispecific, single-chain, and chimeric and humanized molecules having affinity for an ALK1 polypeptide conferred by at least one CDR region of the antibody. In preferred embodiments, the antibody further comprises a label attached thereto and is able to be detected, (e.g., the label can be a radioisotope, fluorescent compound, enzyme or enzyme co-factor).

In certain preferred embodiments, an antibody of the disclosure is a recombinant antibody, particularly a humanized monoclonal antibody or a fully human recombinant antibody.

The adjective “specifically reactive with” as used in reference to an antibody is intended to mean, as is generally understood in the art, that the antibody is sufficiently selective between the antigen of interest (e.g., an ALK1 polypeptide or an ALK1 ligand) and other antigens that are not of interest that the antibody is useful for, at minimum, detecting the presence of the antigen of interest in a particular type of biological sample. In certain methods employing the antibody, a higher degree of specificity in binding may be desirable. For example, an antibody for use in detecting a low abundance protein of interest in the presence of one or more very high abundance protein that are not of interest may perform better if it has a higher degree of selectivity between the antigen of interest and other cross-reactants. Monoclonal antibodies generally have a greater tendency (as compared to polyclonal antibodies) to discriminate effectively between the desired antigens and cross-reacting polypeptides. In addition, an antibody that is effective at selectively identifying an antigen of interest in one type of biological sample (e.g., a stool sample) may not be as effective for selectively identifying the same antigen in a different type of biological sample (e.g., a blood sample). Likewise, an antibody that is effective at identifying an antigen of interest in a purified protein preparation that is devoid of other biological contaminants may not be as effective at identifying an antigen of interest in a crude biological sample, such as a blood or urine sample. Accordingly, in preferred embodiments, the application provides antibodies that have demonstrated specificity for an antigen of interest in a sample type that is likely to be the sample type of choice for use of the antibody.

One characteristic that influences the specificity of an antibody:antigen interaction is the affinity of the antibody for the antigen. Although the desired specificity may be reached with a range of different affinities, generally preferred antibodies will have an affinity (a dissociation constant) of about 10⁻⁶, 10⁻⁷, 10⁻⁸, 10⁻⁹ or less. Given the apparently low binding affinity of TGFβ for ALK1, it is expected that many anti-ALK1 antibodies will inhibit TGFβ binding. However, the GDF5,6,7 group of ligands bind with a K_{D} of approximately 5×10⁻⁸ M and the BMP9,10 ligands bind with a K_{D} of approximately 1×10⁻¹° M. Thus, antibodies of appropriate affinity may be selected to interfere with the signaling activities of these ligands.

In addition, the techniques used to screen antibodies in order to identify a desirable antibody may influence the properties of the antibody obtained. For example, an antibody to be used for certain therapeutic purposes will preferably be able to target a particular cell type. Accordingly, to obtain antibodies of this type, it may be desirable to screen for antibodies that bind to cells that express the antigen of interest (e.g., by fluorescence activated cell sorting). Likewise, if an antibody is to be used for binding an antigen in solution, it may be desirable to test solution binding. A variety of different techniques are available for testing antibody:antigen interactions to identify particularly desirable antibodies. Such techniques include ELISAs, surface plasmon resonance binding assays (e.g., the Biacore binding assay, Bia-core AB, Uppsala, Sweden), sandwich assays (e.g., the paramagnetic bead system of IGEN International, Inc, Gaithersburg, Md.), western blots, immunoprecipitation assays and immunohistochemistry.

In a preferred embodiment, an antibody disclosed herein is an antibody that binds to the mature portion of human BMP9, the amino acid sequence of which is shown below:

(SEQ ID NO: 12)
RS AGAGSHCQKT SLRVNFEDIG WDSWIIAPKE YEAYECKGGC
FFPLADDVTP TKHAIVQTLV HLKFPTKVGK ACCVPTKLSP
ISVLYKDDMG VPTLKYHYEG MSVAECGCR

In an additional embodiment, an antibody disclosed herein is an antibody that binds to the mature portion of human BMP10, the amino acid sequence of which is shown below:

(SEQ ID NO: 13)
NAKG NYCKRTPLYI DFKEIGWDSW IIAPPGYEAY ECRGVCNYPL
AEHLTPTKHA IIQALVHLKN SQKASKACCV PTKLEPISIL
YLDKGVVTYK FKYEGMAVSE CGCR

Additionally, non-antibody proteins that bind to BMP9 or BMP10 may be generated by selection from libraries. A wide variety of technologies are available for selecting random peptides, as well as framework based proteins, that bind to a particular ligand. In general, an approach to identifying a useful non-antibody protein will involve screening or selecting from a library those proteins that bind to BMP9 and/or BMP10 or inhibit a BMP9 or BMP10 activity, such as receptor (e.g., ALK1) binding or cellular signaling (e.g, SMAD 1/5 signaling).

5. Alterations in Antibodies and Fc-Fusion Proteins

The application further provides antibodies and ALK1-Fc fusion proteins that contain engineered or variant Fc regions. Such antibodies and Fc fusion proteins may be useful, for example, in modulating effector functions, such as, antigen-dependent cytotoxicity (ADCC) and complement-dependent cytotoxicity (CDC). Additionally, the modifications may improve the stability of the antibodies and Fc fusion proteins. Amino acid sequence variants of the antibodies and Fc fusion proteins are prepared by introducing appropriate nucleotide changes into the DNA, or by peptide synthesis. Such variants include, for example, deletions from, and/or insertions into and/or substitutions of, residues within the amino acid sequences of the antibodies and Fc fusion proteins disclosed herein. Any combination of deletion, insertion, and substitution is made to arrive at the final construct, provided that the final construct possesses the desired characteristics. The amino acid changes also may alter post-translational processes of the antibodies and Fc fusion proteins, such as changing the number or position of glycosylation sites.

Antibodies and Fc fusion proteins with reduced effector function may be produced by introducing changes in the amino acid sequence, including, but are not limited to, the Ala-Ala mutation described by Bluestone et al., (see WO 94/28027 and WO 98/47531; also see Xu et al., 2000 Cell Immunol 200; 16-26). Thus in certain embodiments, antibodies and Fc fusion proteins of the disclosure containing mutations within the constant region including the Ala-Ala mutation may be used to reduce or abolish effector function. According to these embodiments, antibodies and Fc fusion proteins may comprise a mutation to an alanine at position 234 or a mutation to an alanine at position 235, or a combination thereof. In one embodiment, the antibody or Fc fusion protein comprises an IgG4 framework, wherein the Ala-Ala mutation would describe a mutation(s) from phenylalanine to alanine at position 234 and/or a mutation from leucine to alanine at position 235. In another embodiment, the antibody or Fc fusion protein comprises an IgG1 framework, wherein the Ala-Ala mutation would describe a mutation(s) from leucine to alanine at position 234 and/or a mutation from leucine to alanine at position 235. The antibody or Fc fusion protein may alternatively or additionally carry other mutations, including the point mutation K322A in the CH2 domain (Hezareh et al., 2001; J. Virol. 75: 12161-8).

In particular embodiments, the antibody or Fc fusion protein is modified to either enhance or inhibit complement dependent cytotoxicity (CDC). Modulated CDC activity may be achieved by introducing one or more amino acid substitutions, insertions, or deletions in an Fc region (see, e.g., U.S. Pat. No. 6,194,551). Alternatively or additionally, cysteine residue(s) may be introduced in the Fc region, thereby allowing interchain disulfide bond formation in this region. The homodimeric antibody thus generated may have improved or reduced internalization capability and/or increased or decreased complement-mediated cell killing. See Caron et al.; J. Exp Med. 1992; 176:1191-1195 and Shopes, B. (1992); J. Immunol. 148:2918-2922, WO99/51642, Duncan & Winter Natureb, 1988; 322: 738-40; U.S. Pat. No. 5,648,260; U.S. Pat. No. 5,624,821; and WO94/29351.

6. Methods and Compositions for Treating Renal Cell Carcinoma, Modulating Angiogenesis and Treating Other Disorders

The disclosure provides methods of treating renal cell carcinoma in a mammal by administering to a mammal an effective amount of an ALK1 ECD polypeptide, such as an ALK1-Fc fusion protein, or an antibody disclosed herein, such as an antibody against GDF5, GDF6, GDF7, BMP9, BMP10, or the ECD of ALK1, or nucleic acid antagonists (e.g., antisense or siRNA) of any of the foregoing hereafter collectively referred to as “therapeutic agents” or “ALK1 “antagonist(s).” It is expected that these therapeutic agents are useful in treating renal cell carcinoma as a single agents, or in combination with other RCC therapeutic agents.

In particular, polypeptide therapeutic agents of the present disclosure have several properties that make them particularly attractive as therapeutic agents in treating RCC. For example, unlike most biologic agents, ALK1 ECD polypeptides affect renal cell growth by modulating multiple factors that promote and sustain tumor growth, proliferation and tumor angiogenesis. This is highly relevant in cancers, where a cancer will frequently have mutations associated with multiple distinct signaling pathways that drive for example, tumor growth, proliferation, angiogenesis, and metastasis. Thus, the therapeutic agents disclosed herein are particularly effective in treating tumors such as renal cell carcinomas that are resistant to treatment with a drug that targets a single angiogenic factor (e.g., bevacizumab, which targets VEGF), while at the same time providing the potential to antagonize the activity of ALK1, which is selectively expressed on activated endothelium cells and appears to play an instrumental role in regulating the response of these cells to multiple factors such as BMP9, VEGF, and FGF that drive tumor angiogenesis and cell proliferation.

As demonstrated herein, ALK1-Fc fusion proteins are effective in reducing tumor growth of tumors in vivo in a human RCC xenograft model. Accordingly, it is expected that ALK1 ECD polypeptides such as ALK1-Fc fusion proteins and other therapeutic agents disclosed herein are useful in stand-alone (i.e., single agent) therapy for treating renal cell carcinoma. Additionally, as further disclosed herein, ALK1-Fc fusion protein significantly increases the tumor growth inhibiting activity of sunitinib, the current standard of care in advanced RCC in each of the human RCC xenograft models tested. Accordingly, it is expected that ALK1 ECD polypeptides such as ALK1-Fc fusion proteins and other therapeutic agents disclosed herein are useful in combination therapy with other agents, such as receptor tyrosine kinase inhibitors for treating renal cell carcinoma.

As used herein, the term “treat” or “treatment” refers to contact or administration of an exogenous therapeutic agent, diagnostic agent, or composition to the mammal (e.g., human), subject, cell, tissue, organ, or biological fluid, and can refer, e.g., to therapeutic, pharmacokinetic, diagnostic, research, and experimental methods. “Treating” or “treatment” include the administration of an ALK1 ECD polypeptide, such as an ALK1-Fc fusion protein or other ALK antagonist to prevent or delay the onset of the symptoms, complications, or biochemical indicia of a disease, condition, or disorder, alleviating the symptoms or arresting or inhibiting further development of the disease, condition, or disorder. Treatment can be prophylactic (to prevent or delay the onset of the disease, or to prevent the manifestation of clinical or subclinical symptoms thereof) or therapeutic suppression or alleviation of symptoms after the manifestation of the disease, condition, or disorder. Treatment can be with the ALK1 ECD polypeptide (e.g., ALK1-Fc fusion protein) or other ALK1 antagonist alone, or in combination with one or more additional therapeutic agents. As used herein, the term “mammal” or “subject” refers to a mammalian animal (including but not limited to non-primates such as cows, pigs, horses, sheep, cows, dogs, cats, rats, and mice), more specifically a primate (including but not limited to monkeys, apes, and humans), and even more specifically, a human.

As used herein, the term “amount effective” or “effective amount” (e.g., to treat, etc.) refers to an amount of a therapeutic agent, e.g., an ALK1 ECD polypeptide such as an ALK1-Fc fusion protein, that is sufficient to achieve the desired effect, such as, to alleviate one or more disease symptoms or effects in the treated subject or population, whether by inducing the regression of or inhibiting the progression of such symptom(s) or effects by any clinically measurable degree. The amount of a therapeutic agent that is effective to alleviate any particular disease symptom or effect (also referred to as the “therapeutically effective amount”) or prevent an particular disease symptom or effect (also referred to as the “prophylactically effective amount”) may vary according to factors such as the disease state, age, and weight of the patient, and the ability of the drug to elicit a desired response in the patient. Whether a disease symptom or effect has been alleviated can be assessed by any clinical measurement typically used (e.g., by healthcare providers or laboratory clinicians) to assess the severity or progression status of that symptom or effect.

As used herein, the term “acronym “RTKI” refers to a small-molecule receptor tyrosine kinase inhibitor that binds to and inhibits signaling of VEGFR1, VEGFR2, or VEGFR3. An RTKI can bind to and inhibit receptor tyrosine kinases in addition to a VEGFR, such as PDGFRa, PDGFRb, RET, and c-Met. Likewise, an RTKI can inhibit a different class of kinases and kinases that are not cell surface receptors, such as the serine kinases B-raf kinase and c-raf kinase.

Thus, in one aspect, the disclosure relates to a method of treating renal cell carcinoma (RCC) in a mammal, comprising administering to a mammal that has RCC an effective amount of an RTKI and an ALK1 ECD polypeptide, such as an ALK1-Fc fusion protein or other ALK antagonist disclosed herein. In one aspect, the ALK antagonist is an agent selected from (a) an ALK1 polypeptide comprising a ligand binding portion of the extracellular domain of ALK1, (b) an antibody that hinds to the extracellular domain of human ALK1; (c) an antibody that binds to human BMP9; and (d) an antibody that binds to human BMP10. In some aspects, the ALK1 polypeptide used according to the method comprises a polypeptide having an amino acid sequence that is at least 90% identical to the sequence of amino acids 22.-120 of SEQ ID NO:1. In further aspects, the ALK1 polypeptide further comprises a constant domain of an immunoglobulin. In further aspects the ALK1 polypeptide further comprises an Fc portion of an immunoglobulin and in additional aspects, the Fc portion is an Fc portion of a human IgG1. In other aspects, the ALK1 polypeptide comprises an amino acid sequence that is at least 90% identical to the sequence of SEQ ID NO: 3 or SEQ ID NO:14.

In an additional aspect, the disclosure encompasses a method of treating renal cell carcinoma in a mammal that has RCC and that has undergone a medical procedure to treat RCC. In particular embodiments, the medical procedure is selected from nephron-sparing surgery, nephrectomy, complete nephrectomy and tissue ablation. In further aspects, the treatment is administered to the mammal within 1, 2, 3, 4, 5, 6, or one month after the medical procedure.

In some aspects the antibody used according to the methods of the disclosure bind an epitope within the sequence of amino acids 22-118 of SEQ ID NO:1 and inhibits binding of a ligand selected from GDF5, GDF6, GDF7, BMP9 and BMP 10. In additional aspects, the antibody binds to an epitope within the sequence of amino acids 1-111 of SEQ ID NO:12 and inhibits binding of BMP9 to a receptor. In further aspects, the antibody binds to an epitope within the sequence of amino acids 1-108 of SEQ ID NO:13 and inhibits binding of BMP10 to a receptor.

In some aspects the RTKI used according to the methods of the disclosure is sunitinib.

In other embodiments, the RTKI used according to the methods of the disclosure is not sunitinib.

In some aspects the RTKI used according to the methods of the disclosure is sorafenib. In additional aspects, the RTKI is pazopanib. In additional aspects, the RTKI is axitinib. In another aspect, the RTKI is tivozanib or vandetanib. In additional aspects RTKI used according to the method is an agent selected from: motesanib (AMG-705), vatalanib (PTK787/ZK), samaxanib (SU5416), SU6668, AZD2171, XL184, XL880/GSK1363089, PF-2351066, MGCD265, ZD6474, AEE788, AG-013736, AG-013737, GW786034, and ABT-869. In further aspects the pharmaceutical preparations comprise a VEGF receptor tyrosine kinase inhibitor agent disclosed in International Patent Appl. Publ. Nos. WO97/22596, WO 97/30035, WO 97/32856 or WO 98/13354. In additional aspects, the disclosure relates to a method of treating renal cell carcinoma (RCC) in a mammal, comprising administering to a mammal that has RCC (1) an effective amount of (1) an RTKI, (2) an ALK1 ECD polypeptide, such as an ALK1-Fc fusion protein or other ALK antagonist disclosed, and (3) a mammalian target of rapamycin (mTOR)-targeted inhibitor. As used herein, the term “mTOR-targeted inhibitor” refers to a small-molecule inhibitor that binds to and inhibits signaling of the AKT/mTOR signalling pathway. mTOR-targeted inhibitors, and assays for identifying mTOR-targeted inhibitors that can be used according to the methods of the disclosure are known in the art. In some aspects, the mTOR inhibitor used according to the methods of the invention is everolimus. In other aspects, the mTOR inhibitor is temsirolimus. In additional aspects, the mTOR inhibitor is an agent selected from: WYE354, YE132 (Pfizer), PP30 and PP242, AZD8055, OSI-027, Torin1, BEZ235, XL765, GDC-0980, PF-04691502 and PF-05212384.

In additional aspects, the renal cell carcinoma (RCC) treated according to the methods of the disclosure is a clear cell renal cell carcinoma. In some aspects, the RCC is a TNM stage III disease. In additional aspects, the RCC is a TNM stage IV disease. In additional aspects, the RCC is found within the intrarenal veins. In other aspects, the RCC has invaded the renal sinus. In further aspects, the RCC has metastasized to the adrenal gland or to a lymph node. In further aspects, the RCC has metastasized to the lung, intra-abdominal lymph nodes, bone, brain, or liver.

Thus, according to one aspect, the disclosure relates to a method of treating metastatic renal cell carcinoma (RCC) in a mammal, comprising administering to a mammal having metastatic RCC an effective amount of an RTKI and an ALK1 ECD polypeptide, such as an ALK1-Fc fusion protein or other ALK antagonist disclosed herein. In one aspect, the disclosure encompasses a method of treating renal cell carcinoma in a mammal that has RCC and that has undergone a medical procedure to treat RCC. In particular embodiments, the medical procedure is selected from nephron-sparing surgery, nephrectomy, complete nephrectomy and tissue ablation. In further aspects, the treatment is administered to the mammal within 1, 2, 3, 4, 5, 6, or a month after the medical procedure.

In further aspects, the disclosure is directed to methods of treating a mammal that has received prior treatment with an RCC therapeutic agent. In a further aspect the disclosure encompasses a method of treating renal cell carcinoma in a mammal having previously received an RCC therapeutic agent, the method comprising administering to the mammal an effective amount of an agent selected from: (a) an ALK1 polypeptide comprising a ligand binding portion of the extracellular domain of ALK1; (b) an antibody that binds to the extracellular domain of human ALK1; (c) an antibody that binds to human BMP9; and (d) an antibody that binds to human BMP10. In some aspects, the ALK1 polypeptide used according to the method comprises a polypeptide having an amino acid sequence that is at least 90% identical to the sequence of amino acids 22-120 of SEQ ID NO:1. In further aspects, the ALK1 polypeptide further comprises a constant domain of an immunoglobulin. In further aspects the ALK1 polypeptide further comprises an Fc portion of an immunoglobulin and in additional aspects, the Fc portion is an Fc portion of a human IgG1. In other aspects, the ALK1 polypeptide comprises an amino acid sequence that is at least 90% identical to the sequence of SEQ ID NO: 3 or SEQ ID NO:14.

In some aspects the antibody used according to the methods of the disclosure binds to an epitope within the sequence of amino acids 22-118 of SEQ ID NO:1 and inhibits binding of a ligand selected from GDF5, GDF6, GDF7, BMP9 and BMP 10. In additional aspects, the antibody binds to an epitope within the sequence of amino acids 1-111 of SEQ ID NO:12 and inhibits binding of BMP9 to a receptor. In further aspects, the antibody binds to an epitope within the sequence of amino acids 1-108 of SEQ ID NO:13 and inhibits binding of BMP 10 to a receptor.

In one aspect, the previously received RCC therapeutic agent is an RTKI. In a further aspect, the RTKI is an agent selected from: sunitinib, sorafenib, pazopanib, axitinib, tivozanib and vandetanib. In another aspect, the previously received RCC therapeutic agent is a mammalian target of rapamycin (mTOR)-targeted inhibitor. In a further aspect, the mTOR-targeted inhibitor is an agent selected from: everolimus and temsirolimus. In other aspects, the mTOR inhibitor is an agent selected from: WYE354, YE132 (Pfizer), PP30 and PP242, AZD8055, OSI-027, Torin1, BEZ235, XL765, GDC-0980, PF-04691502 and PF-05212384.

In an additional aspect, the previously received RCC therapeutic agent is a systemic cytokine therapy. In a further aspect, the previously received RCC therapeutic agent is interferon alpha (IFN-α) or interleukin-2 (IL-2).

In some aspects the RTKI used according to the methods of treating a mammal that has received prior treatment with an RCC therapeutic agent is sunitinib.

In other embodiments, the RTKI used according to the methods of treating a mammal that has received prior treatment with an RCC therapeutic agent is not sunitinib.

In some aspects the RTKI used according to the methods of treating a mammal that has received prior treatment with an RCC therapeutic agent is sorafenib. In additional aspects, the RTKI is pazopanib. In additional aspects, the RTKI is axitinib. In another embodiment, the RTKI is tivozanib or vandetanib. In additional aspects RTKI used according to the method is an agent selected from: motesanib (AMG-706), vatalanib (PTK787/ZK), satnaxanib (SU5416), SU6668, AZD2171, XL184, XL880/GSK1363089, PF-2351066, MGCD265, ZD6474, AEE788, AG-013736, AG-013737, GW786034, and ABT-869. In further aspects the pharmaceutical preparations comprise a VEGF receptor tyrosine kinase inhibitor agent disclosed in International Patent Appl. Publ. Nos. WO97/22596, WO 97/30035, WO 97/32856 or WO 98/13354.

In additional aspects, the disclosure relates to a method of treating renal cell carcinoma (RCC) in a mammal having previously received an RCC therapeutic agent, the method comprising administering to the mammal an effective amount of (1) an RTKI, (2) an ALK1 ECD polypeptide, such as an ALK1-Fc fusion protein or other ALK antagonist disclosed, and (3) a mammalian target of rapamycin (mTOR)-targeted inhibitor. mTOR-targeted inhibitors, and assays for identifying mTOR-targeted inhibitors that can be used according to the methods of the disclosure are known in the art. In some aspects, the mTOR inhibitor used according to the methods of the invention is everolimus. In other aspects, the mTOR inhibitor is temsirolimus. In other aspects, the mTOR inhibitor is an agent selected from: WYE354, YE132 (Pfizer), PP30 and PP242, AZD8055, OSI-027, Torin1, BEZ235, XL765, GDC-0980, PF-04691502 and PF-05212384.

In additional aspects, the disclosure relates to a method of treating renal cell carcinoma (RCC) in a mammal having previously received an RCC therapeutic agent wherein the renal cell carcinoma (RCC) treated according to the methods of the disclosure is a clear cell renal cell carcinoma. In some aspects, the RCC is a TNM stage III disease. In additional aspects, the RCC is a TNM stage IV disease. In additional aspects, the RCC is found within the intrarenal veins. In other aspects, the RCC has invaded the renal sinus. In further aspects, the RCC is metastatic renal cell carcinoma. In additional aspects, the RCC has metastasized to the adrenal gland or to a lymph node. In further aspects, the RCC has metastasized to the lung, intra-abdominal lymph nodes, bone, brain, or liver.

In further aspects, the disclosure is directed to methods of treating a mammal that has RCC and that is preparing to undergo a medical procedure to treat RCC. In one aspect, the disclosure encompasses a method of treating renal cell carcinoma in a mammal that has RCC and that is preparing to undergo a medical procedure to treat RCC, the method comprising administering to the mammal an effective amount of an agent selected from: (a) an ALK1 polypeptide comprising a ligand binding portion of the extracellular domain of ALK1; (b) an antibody that binds to the extracellular domain of human ALK1; (c) an antibody that binds to human BMP9; and (d) an antibody that binds to human BMP10. In some aspects, the ALK1 polypeptide used according to the method comprises a polypeptide having an amino acid sequence that is at least 90% identical to the sequence of amino acids 22-120 of SEQ ID NO:1. In further aspects, the ALK1 polypeptide further comprises a constant domain of an immunoglobulin. In further aspects the ALK1 polypeptide further comprises an Fc portion of an immunoglobulin and in additional aspects, the Fc portion is an Fc portion of a human IgG1. In other aspects, the ALK1 polypeptide comprises an amino acid sequence that is at least 90% identical to the sequence of SEQ ID NO: 3 or SEQ ID NO:14. In one aspect, the agent is administered at least, 1, 2, 3, 4, 5, 6, or 7 days before the medical procedure. In another aspect the mammal has received a series of at least 1, 2, 3, or 4 treatments with the agent prior to the operation. In another aspect, the agent is administered prior to a medical procedure selected from: nephron-sparing surgery, nephrectomy, complete nephrectomy and tissue ablation.

In some embodiments, an antibody is administered to treat a mammal that has RCC and that is preparing to undergo a medical procedure to treat RCC. In further embodiments, the administered antibody binds an epitope within the sequence of amino acids 22-118 of SEQ ID NO:1 and inhibits binding of a ligand selected from GDF5, GDF6, GDF7, BMP9 and BMP 10. In additional aspects, the antibody binds to an epitope within the sequence of amino acids 1-111 of SEQ ID NO:12 and inhibits binding of BMP9 to a receptor. In further aspects, the antibody binds to an epitope within the sequence of amino acids 1-108 of SEQ ID NO:13 and inhibits binding of BMP 10 to a receptor.

In some aspects, an RTKI is administered with an ALK-1 antagonist disclosed herein to treat a mammal prior to undergoing a medical procedure. In some aspects, the RTKI used according to the methods of treating a mammal prior to undergoing a medical procedure to treat RCC is sunitinib. In other embodiments, the RTKI used according to the methods of treating a mammal prior to undergoing a medical procedure to treat RCC is not sunitinib.

In some aspects the RTKI used according to the methods of treating a mammal prior to undergoing a medical procedure to treat RCC is sorafenib. In additional aspects, the RTKI is pazopanib. In additional aspects, the RTKI is axitinib. In another embodiment, the RTKI is tivozanib or vandetanib. In additional aspects RTKI used according to the method is an agent selected from: motesanib (AMG-706), vatalanib (PTK787/ZK), samaxanib (SU5416), SU6668, AZD2171, XL184, XL880/GSK1363089, PF-2351066, MGCD265, ZD6474, AEE788, AG-013736, AG-013737, GW786034, and ABT-869. In further aspects the agent comprises an RTKI disclosed in International Patent Appl. Publ. Nos. WO97/22596, WO 97/30035, WO 97/32856 or WO 98/13354.

In some aspects, the disclosure is directed to methods of treating a mammal that has RCC and that is preparing to undergo a medical procedure to treat RCC wherein the method comprises administering to the mammal an effective amount of (1) an RTKI, (2) an ALK1 ECD polypeptide, such as an ALK1-Fc fusion protein or other ALK antagonist disclosed, and (3) a mammalian target of rapamycin (mTOR)-targeted inhibitor. In some aspects, the mTOR inhibitor used according to the methods of the invention is everolimus. In other aspects, the mTOR inhibitor is temsirolimus. In other aspects, the mTOR inhibitor is an agent selected from: WYE354, YE132 (Pfizer), PP30 and PP242, AZD8055, OSI-027, Torin1, BEZ235, XL765, GDC-0980, PF-04691502 and PF-05212384.

In additional aspects, the disclosure relates to a method of treating renal cell carcinoma (RCC) in a mammal having RCC prior to undergoing a medical procedure to treat RCC wherein the renal cell carcinoma (RCC) is a clear cell renal cell carcinoma. In some aspects, the RCC is a TNM stage III disease. In additional aspects, the RCC is a TNM stage IV disease. In additional aspects, the RCC is found within the intrarenal veins. In other aspects, the RCC has invaded the renal sinus. In further aspects, the RCC is metastatic renal cell carcinoma. In additional aspects, the RCC has metastasized to the adrenal gland or to a lymph node. In further aspects, the RCC has metastasized to the lung, intra-abdominal lymph nodes, bone, brain, or liver.

In a further aspect, the disclosure is directed to methods of treating a mammal that has RCC and that has undergone a medical procedure to treat RCC. In one aspect, the disclosure encompasses a method of treating renal cell carcinoma in a mammal that has RCC and that has undergone a medical procedure to treat RCC, the method comprising administering to the mammal an effective amount of an agent selected from: (a) an ALK1 polypeptide comprising a ligand binding portion of the extracellular domain of ALK1; (b) an antibody that binds to the extracellular domain of human ALK1; (c) an antibody that binds to human BMP9; and (d) an antibody that binds to human BMP10. In some aspects, the ALK1 polypeptide used according to the method comprises a polypeptide having an amino acid sequence that is at least 90% identical to the sequence of amino acids 22-120 of SEQ ID NO:1. In further aspects, the ALK1 polypeptide further comprises a constant domain of an immunoglobulin. In further aspects the ALK1 polypeptide further comprises an Fc portion of an immunoglobulin and in additional aspects, the Fc portion is an Fc portion of a human IgG1. In other aspects, the ALK1 polypeptide comprises an amino acid sequence that is at least 90% identical to the sequence of SEQ ID NO: 3 or SEQ ID NO:14. In one aspect, the agent is administered at least, 1, 2, 3, 4, 5, 6, or 7 days after the medical procedure. In another aspect, the agent is administered within one week, one month, or three months of the medical procedure. In another aspect, the agent is administered at least, 1, 2, 3, 4, 5, 6, or 7 days after In another aspect the mammal receives a series of at least 1, 2,3, or 4 treatments with the agent after the operation. In another aspect, the agent is administered after a medical procedure selected from: nephron-sparing surgery, nephrectomy, complete nephrectomy and tissue ablation.

In some embodiments, an antibody is administered to treat a mammal that has RCC and that has undergone a medical procedure to treat RCC. In some aspects the antibody binds to an epitope within the sequence of amino acids 22-118 of SEQ ID NO:1 and inhibits binding of a ligand selected from GDF5, GDF6, GDF7, BMP9 and BMP 10. In additional aspects, the antibody binds to an epitope within the sequence of amino acids 1-111 of SEQ ID NO:12 and inhibits binding of BMP9 to a receptor. In further aspects, the antibody binds to an epitope within the sequence of amino acids 1-108 of SEQ ID NO:13 and inhibits binding of BMP 10 to a receptor.

In some aspects, an RTKI is administered with an ALK-1 antagonist disclosed herein to treat a mammal that has RCC and that has undergone a medical procedure to treat RCC. in some aspects, the RTKI used according to the methods of treating a mammal after undergoing a medical procedure is sunitinib. In other embodiments, the RTKI used according to the methods of treating a mammal after undergoing a medical procedure to treat RCC is not sunitinib. In some aspects the RTKI used according to the methods of treating a mammal after undergoing a medical procedure to treat RCC is sorafenib. In additional aspects, the RTKI is pazopanib. In additional aspects, the RTKI is axitinib. In another embodiment, the RTKI is tivozanib or vandetanib. In additional aspects RTKI used according to the method is an agent selected from: motesanib (AMG-706), vatalanib (PTK787/ZK), samaxanib (SU5416), SU6668, AZD2171, XL184, XL880/GSK1363089, PF-2351066, MGCD265, ZD6474, AEE788, AG-013736, AG-013737, GW786034, and ABT-869. In further aspects the agent comprises an RTKI disclosed in International Patent Appl. Publ. Nos. WO97/22596, WO 97/30035, WO 97/32856 or WO 98/13354.

In additional aspects, the disclosure relates to a method of treating renal cell carcinoma (RCC) in a mammal after undergoing a medical procedure to treat the RCC wherein the method comprises administering to the mammal an effective amount of (1) an RTKI, (2) an ALK1 ECD polypeptide, such as an ALK1-Fc fusion protein or other ALK antagonist disclosed, and (3) a mammalian target of rapamycin (mTOR)-targeted inhibitor. In some aspects, the mTOR inhibitor used according to the methods of the invention is everolimus. In other aspects, the mTOR inhibitor is temsirolimus. In other aspects, the mTOR inhibitor is an agent selected from: WYE354, YE132 (Pfizer), PP30 and PP242, AZD8055, OSI-027, Torin1, BEZ235, XL765, GDC-0980, PF-04691502 and PF-05212384.

In additional aspects, the disclosure relates to a method of treating RCC in a mammal having RCC after undergoing a medical procedure to treat RCC wherein the renal cell carcinoma (RCC) is a clear cell renal cell carcinoma. In some aspects, the RCC is a TNM stage III disease. In additional aspects, the RCC is a TNM stage IV disease. In additional aspects, the RCC is found within the intrarenal veins. In other aspects, the RCC has invaded the renal sinus. In further aspects, the RCC is metastatic renal cell carcinoma. In additional aspects, the RCC has metastasized to the adrenal gland or to a lymph node. In further aspects, the RCC has metastasized to the lung, intra-abdominal lymph nodes, bone, brain, or liver.

In further aspects, the disclosure is directed to methods of treating a mammal that has metastatic RCC. In one aspect, the disclosure encompasses a method of treating metastatic RCC wherein the method comprises administering to the mammal an effective amount of an agent selected from: (a) an ALK1 polypeptide comprising a ligand binding portion of the extracellular domain of ALK1; (b) an antibody that binds to the extracellular domain of human ALK1; (c) an antibody that binds to human BMP9; and (d) an antibody that binds to human BMP10. In some aspects, the ALK1 polypeptide used according to the method comprises a polypeptide having an amino acid sequence that is at least 90% identical to the sequence of amino acids 22-120 of SEQ ID NO: 1. In further aspects, the ALK1 polypeptide further comprises a constant domain of an immunoglobulin. In further aspects the ALK1 polypeptide further comprises an Fc portion of an immunoglobulin and in additional aspects, the Fc portion is an Fc portion of a human IgG1. In other aspects, the ALK1 polypeptide comprises an amino acid sequence that is at least 90% identical to the sequence of SEQ ID NO: 3 or SEQ ID NO:14.

In some embodiments, an antibody is administered to treat a mammal that has metastatic RCC. In further embodiments, the administered antibody binds an epitope within the sequence of amino acids 22-118 of SEQ ID NO:1 and inhibits binding of a ligand selected from GDF5, GDF6, GDF7, BMP9 and BMP 10. In additional aspects, the antibody binds to an epitope within the sequence of amino acids 1-111 of SEQ ID NO:12 and inhibits binding of BMP9 to a receptor. In further aspects, the antibody binds to an epitope within the sequence of amino acids 1-108 of SEQ ID NO:13 and inhibits binding of BMP10 to a receptor.

According to one aspect, the disclosure relates to a method of treating metastatic renal cell carcinoma (RCC) in a mammal, comprising administering to a mammal having metastatic RCC an effective amount of an RTKI and an ALK1 ECD polypeptide, such as an ALK1-Fc fusion protein or other ALK antagonist disclosed herein. In some aspects, the RTKI is sunitinib. In other embodiments, the RTKI is not sunitinib. In some aspects the RTKI is sorafenib. In additional aspects, the RTKI is pazopanib. In additional aspects, the RTKI is axitinib. In another embodiment, the RTKI is tivozanib or vandetanib. In additional aspects RTKI is an agent selected from: motesanib (AMG-706), vatalanib (PTK787/ZK), samaxanib (SU5416), SU6668, AZD2171, XL184, XL880/GSK1363089, PF-2351066, MGCD265, ZD6474, AEE788, AG-013736, AG-013737, GW786034, and ABT-869. In further aspects the agent comprises an RTKI disclosed in International Patent Appl. Publ. Nos. WO97/22596, WO 97/30035, WO 97/32856 or WO 98/13354.

In some aspects, the disclosure is directed to methods of treating a mammal that has metastatic RCC wherein the method comprises administering to the mammal an effective amount of (1) an RTKI, (2) an ALK1 ECD polypeptide, such as an ALK1-Fc fusion protein or other ALK antagonist disclosed, and (3) a mammalian target of rapamycin (mTOR)-targeted inhibitor. In some aspects, the mTOR inhibitor used according to the methods of the invention is everolimus. In other aspects, the mTOR inhibitor is temsirolimus. In other aspects, the mTOR inhibitor is an agent selected from: WYE354, YE132 (Pfizer), PP30 and PP242, AZD8055, OSI-027, Torin1, BEZ235, XL765, GDC-0980, PF-04691502 and PF-05212384.

The disclosure also provides methods of inhibiting angiogenesis in a mammal by administering to a mammal an effective amount of an ALK1 ECU polypeptide, such as an ALK1-Fc fusion protein, or other “therapeutic agent” or “ALK1 “antagonist” as disclosed herein. It is expected that these therapeutic agents will also be useful in inhibiting angiogenesis in bones and joints, and in tumors, particularly tumors associated with bones and joints.

Angiogenesis associated diseases include, but are not limited to, angiogenesis-dependent cancer, including, for example, solid tumors, blood born tumors such as leukemias, and tumor metastases; benign tumors, for example hemangiomas, acoustic neuromas, neurofibromas, trachomas, and pyogenic granulomas; rheumatoid arthritis; psoriasis; rubeosis; Osler-Webber Syndrome; myocardial angiogenesis; plaque neovascularization; telangiectasia; hemophiliac joints; and angiofibroma.

In particular, polypeptide therapeutic agents of the present disclosure are useful for treating or preventing a cancer (tumor), and particularly such cancers as are known to rely on angiogenic processes to support growth. Unlike most anti-angiogenic agents, ALK1 ECD polypeptides affect angiogenesis that is stimulated by multiple factors. This is highly relevant in cancers, where a cancer will frequently acquire multiple factors that support tumor angiogenesis. Thus, the therapeutic agents disclosed herein will be particularly effective in treating tumors that are resistant to treatment with a drug that targets a single angiogenic factor (e.g., bevacizumab, which targets VEGF). As demonstrated herein, an ALK1-Fc fusion protein is effective in reducing the pathological effects of melanoma, lung cancer and multiple myeloma. Multiple myeloma is widely recognized as a cancer that includes a significant angiogenic component. Accordingly, it is expected that ALK1-Fc fusion proteins and other therapeutic agents disclosed herein will be useful in treating multiple myeloma and other tumors associated with the bone. As demonstrated herein, therapeutic agents disclosed herein may be used to ameliorate the bone damage associated with multiple myeloma, and therefore may be used to ameliorate bone damage associated with bone metastases of other tumors, such as breast or prostate tumors. As noted herein, the GDF5-7 ligands are highly expressed in bone, and, while not wishing to be limited to any particular mechanism, interference with these ligands may disrupt processes that are required for tumor development in bone.

In some aspects, the disclosure is directed to methods of inhibiting angiogenesis in a mammal having a condition for which angiogenesis inhibition is desirable, wherein the method comprises administering to the mammal an effective amount of an agent selected from: (a) an ALK1 polypeptide comprising a ligand binding portion of the extracellular domain of ALK1; (b) an antibody that binds to the extracellular domain of human ALK1; (c) an antibody that binds to human BMP9; and (d) an antibody that binds to human BMP10. In some aspects, the ALK1 polypeptide used according to the method comprises a polypeptide having an amino acid sequence that is at least 90% identical to the sequence of amino acids 22-120 of SEQ ID NO:1. In further aspects, the ALK1 polypeptide further comprises a constant domain of an immunoglobulin. In further aspects the ALK1 polypeptide further comprises an Fc portion of an immunoglobulin and in additional aspects, the Fc portion is an Fc portion of a human IgG1. In other aspects, the ALK1 polypeptide comprises an amino acid sequence that is at least 90% identical to the sequence of SEQ ID NO: 3 or SEQ ID NO:14.

In some embodiments, an antibody is administered to inhibit angiogenesis in a mammal. In further embodiments, the administered antibody binds an epitope within the sequence of amino acids 22-118 of SEQ ID NO:1 and inhibits binding of a ligand selected from GDF5, GDF6, GDF7, BMP9 and BMP 10. In additional aspects, the antibody binds to an epitope within the sequence of amino acids 1-111 of SEQ ID NO:12 and inhibits binding of BMP9 to a receptor. In further aspects, the antibody binds to an epitope within the sequence of amino acids 1-108 of SEQ ID NO:13 and inhibits binding of BMP10 to a receptor.

In some aspects, an RTKI is administered with an ALK-1 antagonist disclosed herein to inhibit angiogenesis in a mammal. In some aspects, the RTKI is sunitinib. In other embodiments, the RTKI is not sunitinib. In some aspects the RTKI is sorafenib. In additional aspects, the RTKI is pazopanib. In additional aspects, the RTKI is axitinib. In another embodiment, the RTKI is tivozanib or vandetanib. In additional aspects RTKI is selected from: motesanib (AMG-706), vatalanib (PTK787/ZK), samaxanib (SU5416), SU6668, AZD2171, XL184, XL880/GSK1363089, PF-2351066, MGCD265, ZD6474, AEE788, AG-013736, AG-013737, GW786034, and ABT-869. In further aspects the RTKI disclosed in International Patent Appl. Publ. Nos. WO97/22596, WO 97/30035, WO 97/32856 or WO 98/13354.

In some aspects, the disclosure is directed to methods of inhibiting angiogenesis wherein the method comprises administering to a mammal an effective amount of (1) an RTKI, (2) an ALK1 ECD polypeptide, such as an ALK1-Fc fusion protein or other ALK antagonist disclosed, and (3) a mammalian target of rapamycin (mTOR)-targeted inhibitor. In some aspects, the mTOR inhibitor used according to the methods of the invention is everolimus. In other aspects, the mTOR inhibitor is temsirolimus. In other aspects, the mTOR inhibitor is an agent selected from: WYE354, YE132 (Pfizer), PP30 and PP242, AZD8055, OSI-027, Torin1, BEZ235, XL765, GDC-0980, PF-04691502 and PF-05212384.

According to the present disclosure, the antiangiogenic agents described herein may be used in combination with other compositions and procedures for the treatment of diseases. For example, a tumor may be treated conventionally with surgery, radiation or chemotherapy combined with the ALK1 or ALK1 ligand antagonist and then the antagonist may be subsequently administered to the patient to extend the dormancy of micrometastases and to stabilize any residual primary tumor.

Angiogenesis-inhibiting agents can also be given prophylactically to individuals known to be at high risk for developing new or re-current cancers. Accordingly, an aspect of the disclosure encompasses methods for prophylactic prevention of cancer in a subject, comprising administrating to the subject an effective amount of an ALK1 or ALK1 ligand antagonist and/or a derivative thereof, or another angiogenesis-inhibiting agent of the present disclosure.

As demonstrated herein, ALK1-Fc is effective for diminishing the phenotype of a murine model of rheumatoid arthritis. Accordingly, therapeutic agents disclosed herein may be used for the treatment of rheumatoid arthritis and other types of bone or joint inflammation.

Certain normal physiological processes are also associated with angiogenesis, for example, ovulation, menstruation, and placentation. The angiogenesis inhibiting proteins of the present disclosure are useful in the treatment of disease of excessive or abnormal stimulation of endothelial cells. These diseases include, but are not limited to, intestinal adhesions, atherosclerosis, scleroderma, and hypertrophic scars, i.e., keloids. They are also useful in the treatment of diseases that have angiogenesis as a pathologic consequence such as cat scratch disease (Rochele minalia quintosa) and ulcers (Helicobacter pylori).

General angiogenesis inhibiting proteins can be used as a birth control agent by reducing or preventing uterine vascularization required for embryo implantation. Thus, the present disclosure provides an effective birth control method when an amount of the inhibitory protein sufficient to prevent embryo implantation is administered to a female. In one aspect of the birth control method, an amount of the inhibiting protein sufficient to block embryo implantation is administered before or after intercourse and fertilization have occurred, thus providing an effective method of birth control, possibly a “morning after” method. While not wanting to be bound by this statement, it is believed that inhibition of vascularization of the uterine endometrium interferes with implantation of the blastocyst. Similar inhibition of vascularization of the mucosa of the uterine tube interferes with implantation of the blastocyst, preventing occurrence of a tubal pregnancy. It is also believed that administration of angiogenesis inhibiting agents of the present disclosure will interfere with normal enhanced vascularization of the placenta, and also with the development of vessels within a successfully implanted blastocyst and developing embryo and fetus.

Administration methods may include, but are not limited to, pills, injections (intravenous, subcutaneous, intramuscular), suppositories, vaginal sponges, vaginal tampons, and intrauterine devices. In certain embodiments, one or more therapeutic agents can be administered, together (simultaneously) or at different times (sequentially). In addition, therapeutic agents can be administered with another type of compound for treating cancer or for inhibiting angiogenesis. In certain embodiments, the subject methods of the disclosure can be used alone. Alternatively, the subject methods may be used in combination with other conventional anti-cancer therapeutic approaches directed to treatment or prevention of proliferative disorders (e.g., tumor). For example, such methods can be used in prophylactic cancer prevention, prevention of cancer recurrence and metastases after surgery, and as an adjuvant of other conventional cancer therapy. The present disclosure recognizes that the effectiveness of conventional cancer therapies (e.g., chemotherapy, radiation therapy, phototherapy, immunotherapy, and surgery) can be enhanced through the use of a subject polypeptide therapeutic agent.

A wide array of conventional compounds have been shown to have anti-neoplastic activities. These compounds have been used as pharmaceutical agents in chemotherapy to shrink solid tumors, prevent metastases and further growth, or decrease the number of malignant cells in leukemic or bone marrow malignancies. Although chemotherapy has been effective in treating various types of malignancies, many anti-neoplastic compounds induce undesirable side effects. It has been shown that when two or more different treatments are combined, the treatments may work synergistically and allow reduction of dosage of each of the treatments, thereby reducing the detrimental side effects exerted by each compound at higher dosages. In other instances, malignancies that are refractory to a treatment may respond to a combination therapy of two or more different treatments.

When a polypeptide therapeutic agent disclosed herein is administered in combination with another conventional anti-neoplastic agent, either concomitantly or sequentially, such therapeutic agent may enhance the therapeutic effect of the anti-neoplastic agent or overcome cellular resistance to such anti-neoplastic agent. This allows decrease of dosage of an anti-neoplastic agent, thereby reducing the undesirable side effects, or restores the effectiveness of an anti-neoplastic agent in resistant cells.

The methods of the disclosure also include co-administration with other medicaments that are used to treat conditions of the eye. When administering more than one agent or a combination of agents and medicaments, administration can occur simultaneously or sequentially in time. The therapeutic agents and/or medicaments may be administered by different routes of administration or by the same route of administration.

7. Formulations and Effective Doses

The therapeutic agents described herein may be formulated into pharmaceutical compositions. Pharmaceutical compositions for use in accordance with the present disclosure may be formulated in conventional manner using one or more physiologically acceptable carriers or excipients. Such formulations will generally be substantially pyrogen free, in compliance with most regulatory requirements.

In certain embodiments, the therapeutic method of the disclosure includes administering the composition systemically, or locally as an implant or device. When administered, the therapeutic composition for use in this disclosure is in a pyrogen-free, physiologically acceptable form. Therapeutically useful agents other than the ALK1 signaling antagonists which may also optionally be included in the composition as described above, may be administered simultaneously or sequentially with the subject compounds (e.g., ALK1 ECD polypeptides or any of the antibodies disclosed herein) in the methods disclosed herein.

Typically, protein therapeutic agents disclosed herein will be administered parentally, and particularly intravenously or subcutaneously. Pharmaceutical compositions suitable for parenteral administration may comprise one or more ALK1 ECD polypeptides or other antibodies in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

The compositions and formulations may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

### EXAMPLES

### Example 1

### Expression of ALK1-Fc Fusion Proteins

Applicants constructed a soluble ALK1 fusion protein that has the extracellular domain of human ALK1 fused to a human Fc or mouse ALK1 fused to a murine Fc domain with a minimal linker in between. The constructs are referred to as hALK1-Fc and mALK1-Fc, respectively.

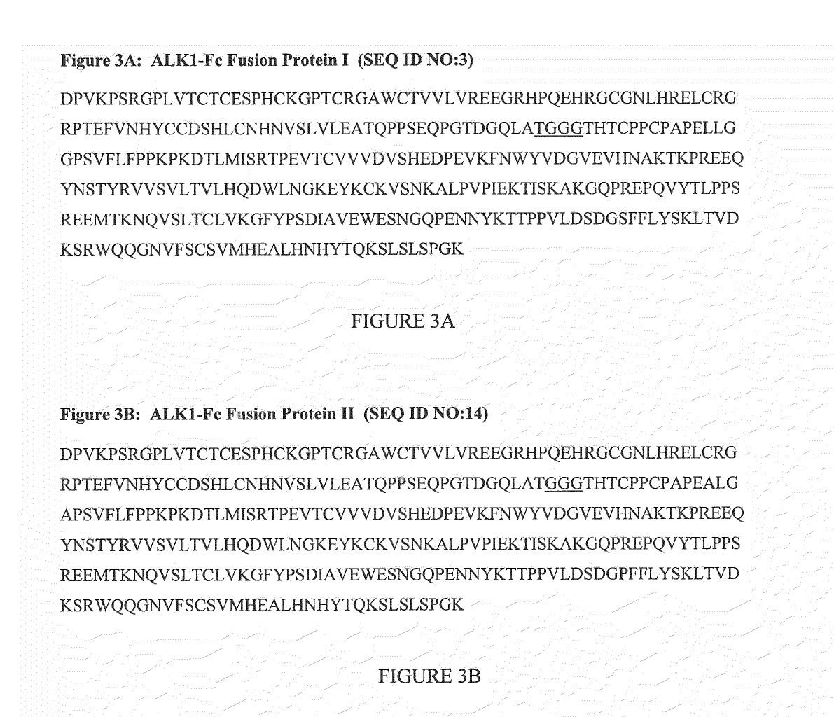

hALK1-Fc is shown as purified from CHO cell lines in FIG. 3B (SEQ ID NO: 14). Notably, while the conventional C-terminus of the extracellular domain of human ALK1 protein is amino acid 118 of SEQ ID NO:1, we have determined that it is desirable to avoid having a domain that ends at a glutamine residue. Accordingly, the portion of SEQ ID NO:14 that derives from human ALK1 incorporates two residues c-terminal to Q118, a leucine and an alanine. The disclosure therefore provides ALK1 ECD polypeptides (including Fc fusion proteins) having a c-terminus of the ALK1 derived sequence that is anywhere from 1 to 5 amino acids upstream (113-117 relative to SEQ ID NO:1) or downstream (119-123) of Q118.

The hALK1-Fc and mALK1-Fc proteins were expressed in CHO cell lines. Three different leader sequences were considered:

(i) Honey bee mellitin (HBML):
(SEQ ID NO: 7)
MKFLVNVALVFMVVYISYIYA
(ii) Tissue Plasminogen Activator (TPA):
(SEQ ID NO: 8)
MDAMKRGLCCVLLLCGAVFVSP
(iii) Native:
(SEQ ID NO: 9)
MTLGSPRKGLLMLLMALVTQG.

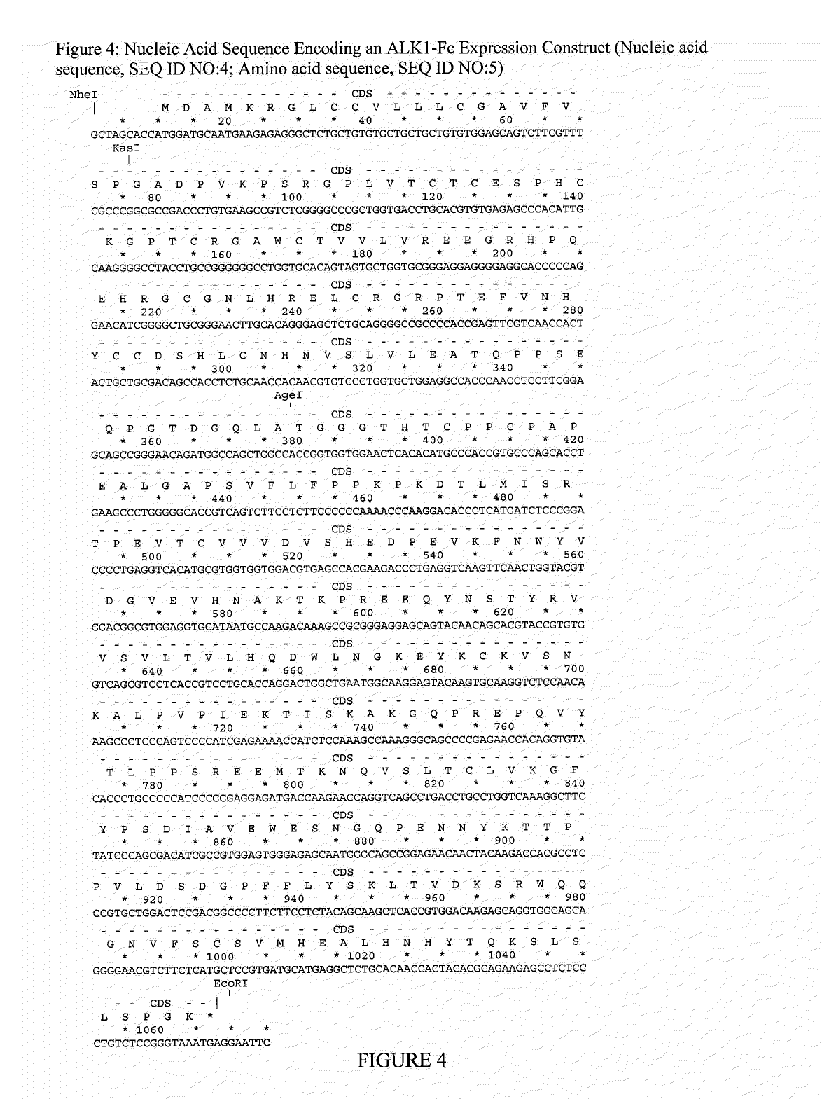

The selected form employs the TPA leader and has the unprocessed amino acid sequence shown in FIG. 4 (SEQ ID NO:5).

This polypeptide is encoded by the nucleic acid sequence shown in FIG. 4 (SEQ ID NO:4).

Purification can be achieved by a series of column chromatography steps, including, for example, three or more of the following, in any order: protein A chromatography, Q sepharose chromatography, phenylsepharose chromatography, size exclusion chromatography, and cation exchange chromatography. The purification can be completed with viral filtration and buffer exchange. The hALK1-Fc protein was purified to a purity of >98% as determined by size exclusion chromatography and >95% as determined by SDS PAGE.

In the course of protein production and purification, we observed that hALK1-Fc tends to be expressed in a mixture of dimers and higher order aggregates which, while appearing pure under denaturing, reducing conditions (e.g., reducing SDS-PAGE), are problematic for administration to a patient. The aggregates may be immunogenic or poorly bioavailable, and because of their heterogeneity, these aggregates make it difficult to characterize the pharmaceutical preparation at a level that is desirable for drug development. Thus, various approaches were tested to reduce the amount of aggregate in final preparations.

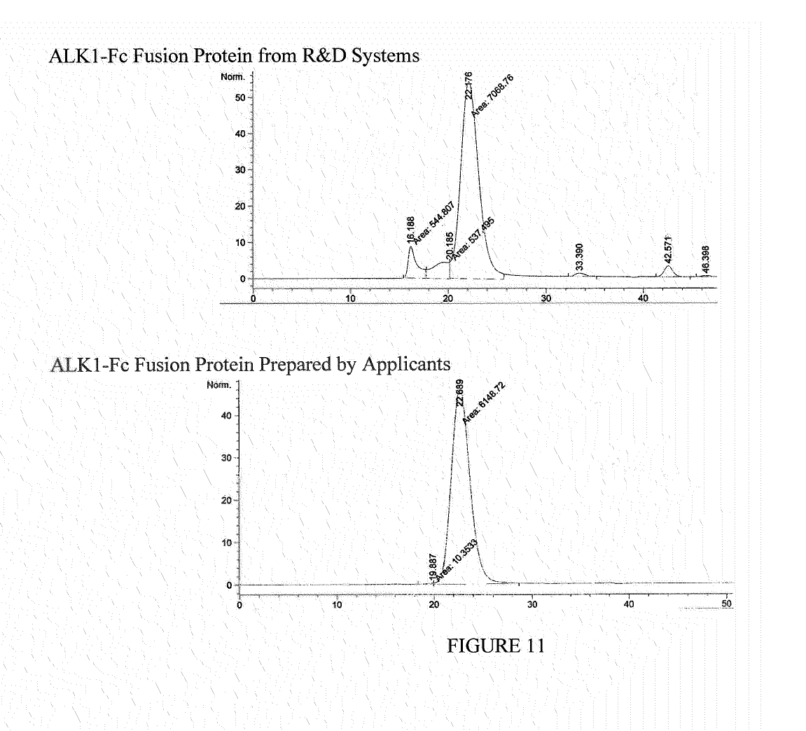

In one approach, a number of different cell culture media were tested. IS CHO-CD (Cat. No. 91119, Irvine Scientific, Santa Ana, Calif.) showed a remarkable reduction in the production of aggregated products, while maintaining high level production of the hALK1-Fc. Additionally, elution of the material from a hydrophobic interaction column (e.g., phenylsepharose) at a pH of 8.0 resulted in further resolution of the aggregated product. The resulting material is comprised of greater than 99% dimers. A comparison to an ALK1-Fc fusion protein sold by R&D Systems (cat. no. 370-AL, Minneapolis, Minn.) shows that this protein, produced in NSO cells, is 84% dimers, with the remaining protein appearing as high molecular weight species by size exclusion chromatography. A comparison of the sizing column profile for the preparations is shown in FIG. 11. Having identified aggregate formation as a significant problem in ALK1-Fc production, it is expected that other approaches may be developed, including approaches that involve additional purification steps (although such approaches may result in lower yield of purified protein).

### Example 2

### Identification of ALK1-Fc Ligands

ALK1 is a type 1 receptor for ligands of the TGFβ family. Multiple members of the TGFβ family were tested for binding to a human ALK1-Fc fusion protein, using a Biacore™ system. TGFβ itself, GDF8, GDF11, BMP2 and BMP4 all failed to show substantial binding to the hALK1-Fc protein, while BMP2 and BMP4 showed only limited binding. In contrast, GDF5 and GDF7 displayed significant binding, with K_{D} values of approximately 5×10⁻⁸ M in both cases. Based on the structural similarity of GDF5 and GDF7 to GDF6, it is expected that GDF6 will bind the fusion protein with similar affinity. The highest binding affinity to hALK1-Fc was observed for BMP9, with K_{D} values ranging from 1×10⁻¹⁹ to 2×10⁻⁹, and BMP10, with a K_{D} of approximately 3×10⁻⁹

### Example 3

### Characterization of ALK1-Fc and Anti-ALK1 Antibody Effects on Endothelial Cells

Using a luciferase reporter construct under the control of four sequential consensus SBE sites (SBE4-luc), which are responsive to Smad1/5/8-mediated signaling, we measured BMP-9 mediated activity in the presence and absence of hALK1-Fc drug or neutralizing ALK1 specific monoclonal antibody in HMVEC cells. HMVEC cells were stimulated with rhBMP-9 (50 ng/ml), which induced Smad1/5/8-mediated transcriptional activation, evidenced here by the increase in SBE4-luc modulated transcriptional upregulation. When added, the hALK1-Fc compound (10 μg/ml) or antibody (10 μg/ml) diminished this transcriptional response, each by nearly 60%, indicating that the presence of ALK1-Fc significantly reduces BMP9 signaling, and moreover, that the BMP9 signaling is related to ALK1 activity.

Activation of SMAD phosphorylation is commonly used to assay activation of upstream activin receptors. ALK1 is known to modulate phosphorylation of SMAD proteins 1,5 and 8 upon activation by its ligand. Here, we added rhBMP-9 (50 ng/ml) to initiate SMAD phosphorylation in HUVEC cells, a human endothelial cell line which innately expresses ALK1 receptor, over a timecourse of 30 minutes. Phosphorylation of SMAD 1/5/8 was seen 5 minutes after treatment of cells with ligand and phosphorylation was maintained for the entirety of the 30 minute period. In the presence of relatively low concentrations of hALK1-Fc (250 ng/ml), SMAD 1/5/8 phosphorylation was reduced, confirming that this agent inhibits Smad1/5/8 activation in endothelial cells.

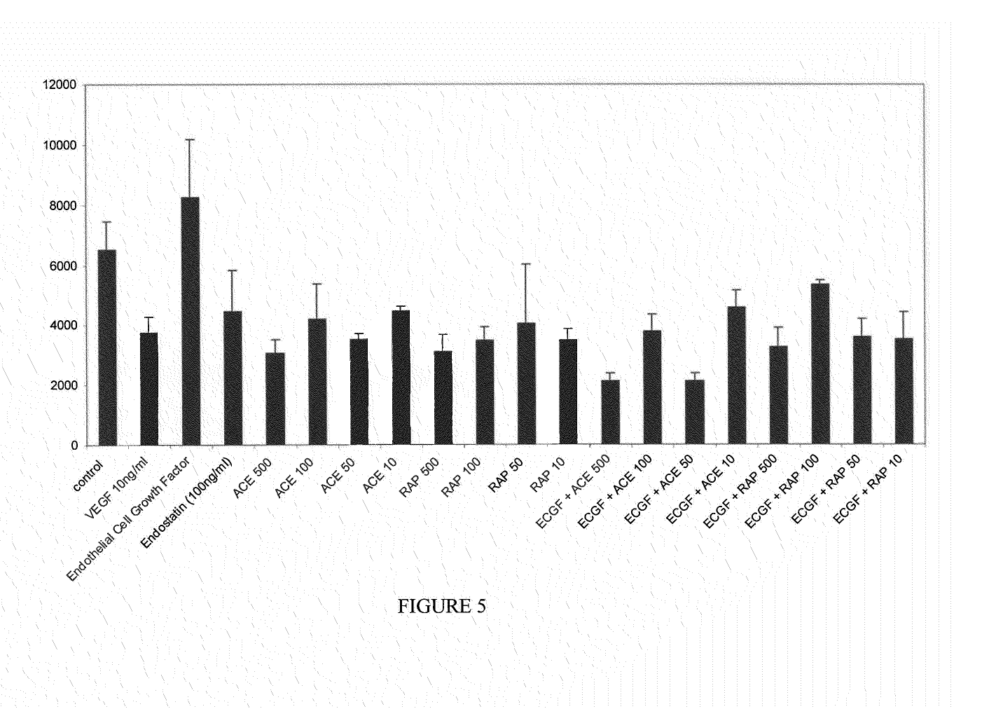

In order to evaluate the angiogenic effect of ALK1-Fc in an in vitro system, we assayed the effectiveness of the compound in reducing tube formation of endothelial cells on a Matrigel substrate. This technique is commonly used to assess neovascularization, giving both rapid and highly reproducible results. Endothelial Cell Growth Supplement (ECGS) is used to induce the formation of microvessels from endothelial cells on Matrigel, and the efficacy of anti-angiogenic compounds are then gauged as a reduction of cord formation in the presence of both the drug and ECGS over an 18 hour timecourse. As expected, addition of ECGS (200 ng/ml) induced significant cord formation, as compared to the negative control (no treatment added), which indicates basal levels of endothelial cell cord formation produced on Matrigel substrate (FIG. 5). Upon addition of either hALK1-Fc (100 ng/ml) or mALK1-Fc (100 ng/ml), cord formation was visibly reduced. Final quantification of vessel length in all samples revealed that every concentration of hALK1-Fc or mALK1-Fc reduced neovascularization to basal levels. Additionally, hALK1-Fc and mALK1-Fc in the presence of the strongly pro-angiogenic factor ECGS maintained strong inhibition of neovascularization demonstrating even more potent anti-angiogenic activity than the negative control endostatin (100 ng/ml).

### Example 4

### CAM Assays

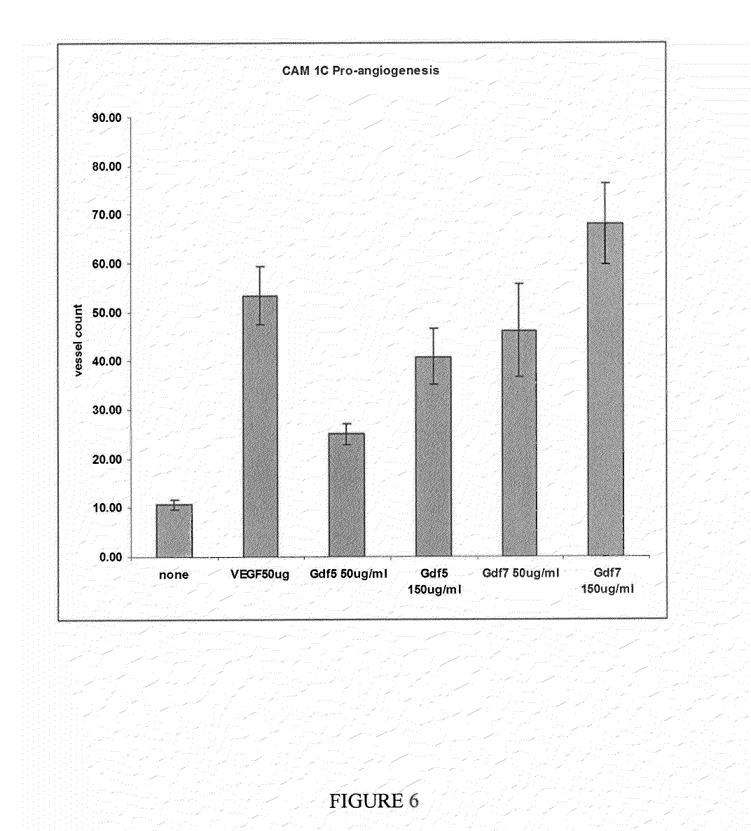

VEGF and FGF are well-known to stimulate angiogenesis. A CAM (chick chorioallantoic membrane) assay system was used to assess the angiogenic effects of GDF7. As shown in FIG. 6, GDF7 stimulates angiogenesis with a potency that is similar to that of VEGF. Similar results were observed with GDF5 and GDF6.

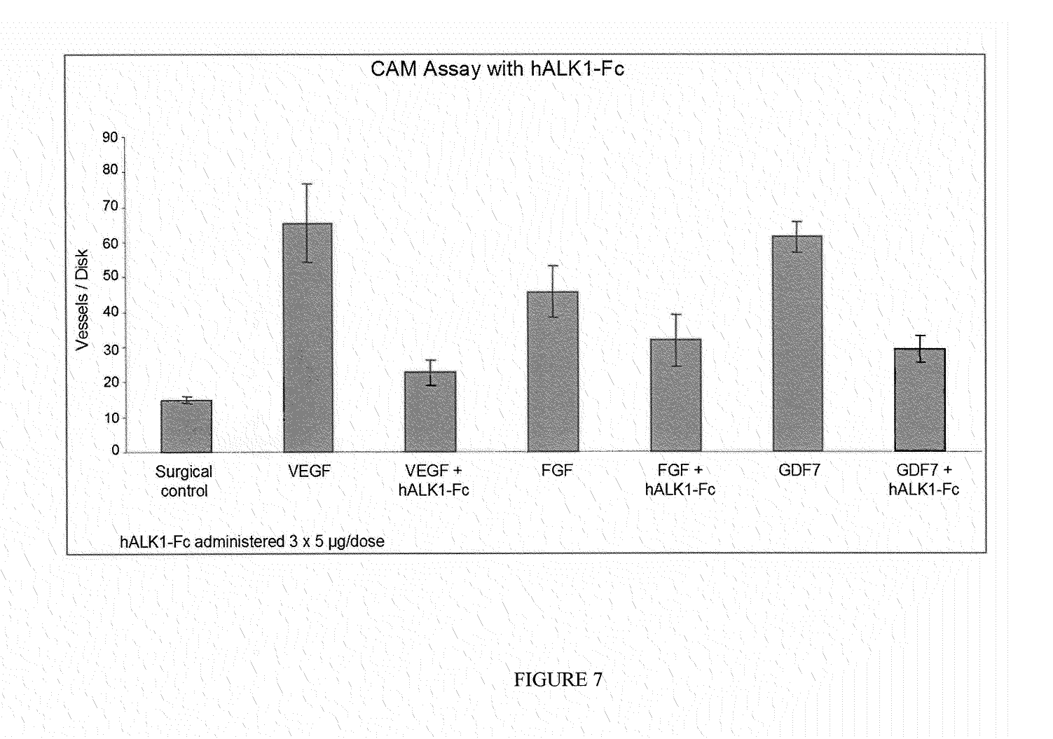

ALK1-Fc fusions were tested for anti-angiogenic activity in the CAM assay. These fusion proteins showed a potent anti-angiogenic effect on angiogenesis stimulated by VEGF, FGF and GDF7. See FIG. 7. BMP9 and PDGF showed a relatively poor capability to induce angiogenesis in this assay, but such angiogenesic effect of these factors was nonetheless inhibited by ALK1.

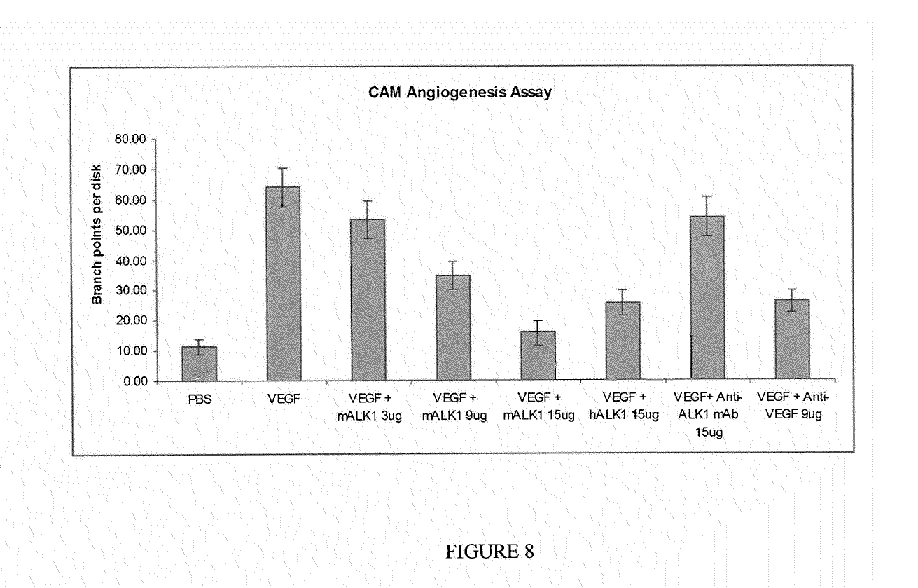

ALK1-Fc proteins and a commercially available, anti-angiogenic anti-VEGF monoclonal antibody were compared in the CAM assay. The ALK1-Fc proteins had similar potency as compared to anti-VEGF. The anti-VEGF antibody bevacizumab is currently used in the treatment of cancer and macular degeneration in humans. See FIG. 8.

Interestingly, an anti-ALK1 antibody (R&D Systems) failed to significantly inhibit angiogenesis in this assay system. We expect that this may reflect the difference in the ALK1 sequence in different species.

### Example 5

### Mouse Corneal Micropocket Assay

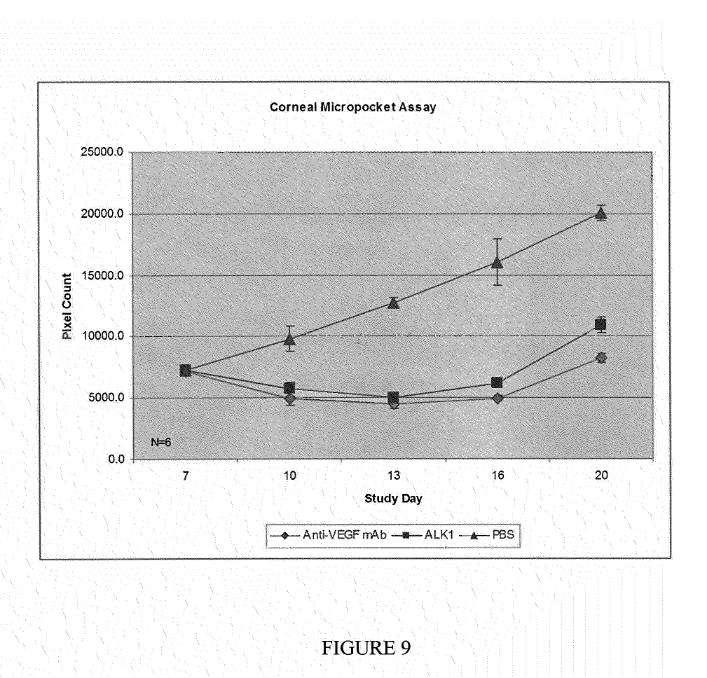

The mouse corneal micropocket assay was used to assess the effects of ALK1-Fc on angiogenesis in the mouse eye. hALK1-Fc, administered intraperitoneally, significantly inhibited ocular angiogenesis. As shown in FIG. 9, hALK1-Fc inhibited ocular angiogenesis to the same degree as anti-VEGF. hALK1-Fc and anti-VEGF were used at identical weight/weight dosages. Similar data were obtained when a Matrigel plug impregnated with VEGF was implanted in a non-ocular location.

These data demonstrate that high affinity ligands for ALK1 promote angiogenesis and that an ALK1-Fc fusion protein has potent anti-angiogenic activity. The ligands for ALK1 fall into two categories, with the GDF5,6,7 grouping having an intermediate affinity for ALK1 and the BMP9,10 grouping having a high affinity for ALK1.

GDF5, 6 and 7 are primarily localized to bone and joints, while BMP9 is circulated in the blood. Thus, there appears to be a pro-angiogenic system of the bones and joints that includes ALK1, GDF5, 6 and 7 and a systemic angiogenic system that includes ALK1 and BMP9 (and possibly BMP10).

### Example 6

### Murine Model of Rheumatoid Arthritis

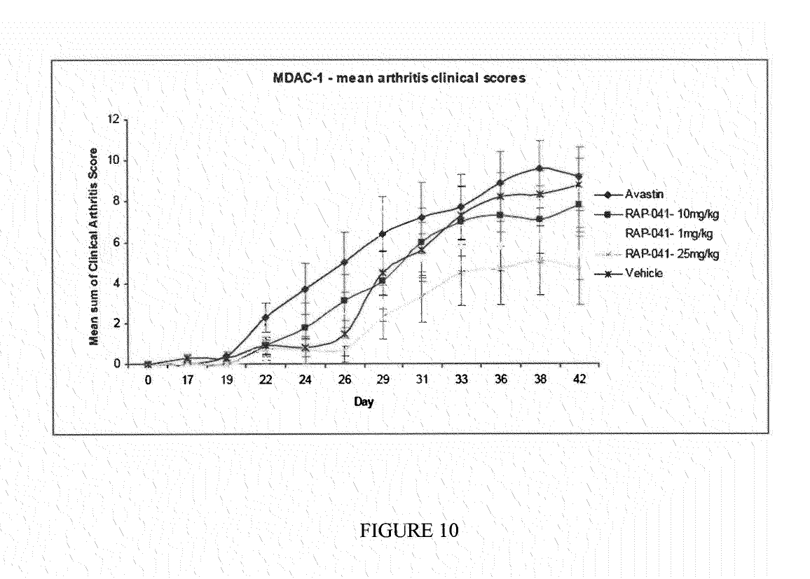

The murine collagen-induced arthritis model is a well-accepted model of rheumatoid arthritis. In this study, groups of 10 mice were treated with vehicle, anti-VEGF (bevacizumab—as a negative control, because bevacizumab does not inhibit murine VEGF), or doses of mALK1-Fc (“RAP-041”) at 1 mg/kg, 10 mg/kg or 25 mg/kg. Following the collagen boost on day 21 arthritic scores (see FIG. 10) and paw swelling steadily increased in all groups, peaking around day 38. Mice treated with mALK1-Fc (“RAP-041”) showed reduced scores for both characteristics, particularly at the highest dose (25 mg/kg), although the reduction did not achieve statistical significance. Nonetheless, a dose-related trend is apparent.

By study termination at day 42 the incidence of arthritis had reached 10/10 in the vehicle control treated mice, 9/10 in the bevacizumab treated mice, 8/10 in the mALK1-Fc at 1 mg/kg treated group and 9/10 in the mALK1-Fc 10 mg/kg treated group. In the mALK1-Fc 25 mg/kg treated group disease incidence was lower at 6/10.

### Example 7

### ALK1-Fc Reduces Tumor Angiogenesis in a CAM Assay

Tumors, as with any tissue, have a basic nutrient and oxygen requirement. Although small tumors are capable of acquiring adequate amounts via diffusion from neighboring blood vessels, as the tumor increases in size, it must secure nutrients by recruiting and maintaining existing capillaries. In order to test the capacity of ALK1-Fc proteins to limit tumor growth through vessel inhibition, we tested varying concentrations of mALK1-Fc in a melanoma explant CAM assay. As with CAM assays described above, small windows were made in the surface of each egg through which 5×1.0⁵ B16 melanoma cells were implanted. Eggs were then treated daily with 0.02 mg/ml mALK1-Fc, 0.2 mg/ml mALK1-Fc, or left untreated for a period of a week. At the end of the experiment, tumors were carefully removed, weighed and digital images were captured. Tumors originating from CAMs treated with mALK1-Fc showed a significant decrease in size as compared: to untreated CAM tumors. Quantification of tumor weight demonstrated that weight of tumors treated daily with either 0.02 mg/ml or 0.2 mg/ml mALK1-Fc showed a reduction of 65% and 85% compared to the untreated CAMs (FIG. 6E). In conclusion, neovascularization and tumor growth was significantly suppressed upon addition of ALK1-Fc in a dose-responsive manner, indicating that ALK1-Fc is a powerful anti-angiogenic agent.

### Example 8

### Lung Cancer Experimental Model

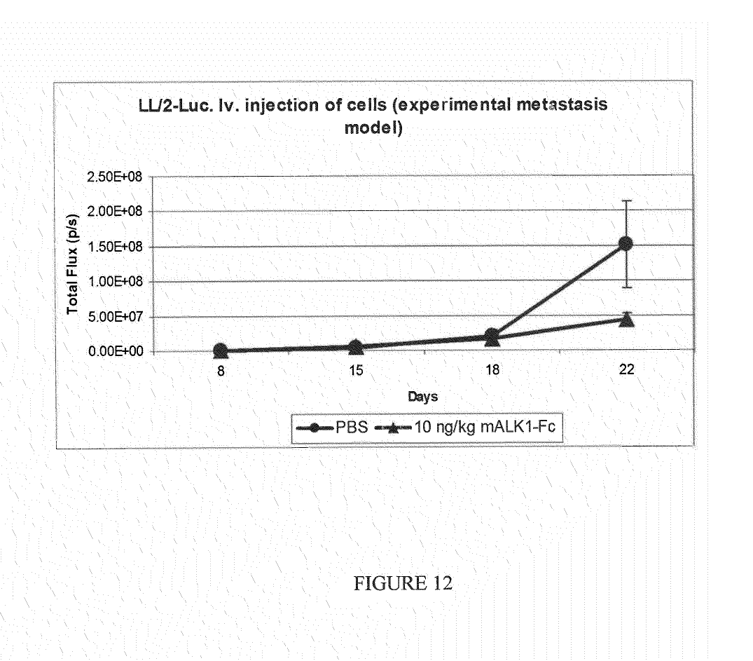

To farther confirm the effects of ALK1-Fc on tumor progression, a mouse model of lung cancer was tested. Fluorescently labeled murine Lewis lung cancer cells (LL/2-luc) were administered to albino Black 6 mice through the tail vein. On the same day, the mice began treatment with either PBS control (n=7) or 10 mg/kg mALK1-Fc (n=7) administered intraperitoneally. In-life fluorescent imaging showed substantial development of tumors localized to the lungs in the control mice, to the point that the mice became moribund and had to be sacrificed by day 22 post-implantation. By contrast, the ALK1-Fc treated mice showed a substantially delayed growth of lung tumors and exhibited 100% survival as of day 22. See FIG. 12.

These data demonstrate that ALK1-Fc has substantial effect on tumor growth in a mouse model of lung cancer and provides a survival benefit.

### Example 9

### BMP9 and Anti-BMP9, Effects on Angiogenesis

A CAM (chick chorioallantoic membrane) assay system was used to assess the angiogenic effects of recombinant human BMP9 (rhB9) and anti-BMP9 monoclonal antibody (mabB9) (R&D Systems, Minneapolis, Minn., Cat. No. MAB3209). This antibody is known to neutralize BMP9/ALK1 signaling. See, e.g., Scharpfenecker et al., J Cell Sci. 2007 Mar. 15; 120(Pt 6):964-72; David et al., (2007); Blood March 1; 109(5):1953-61; David et al., Circ. Res. 2008 Apr. 25; 102(8):914-22.

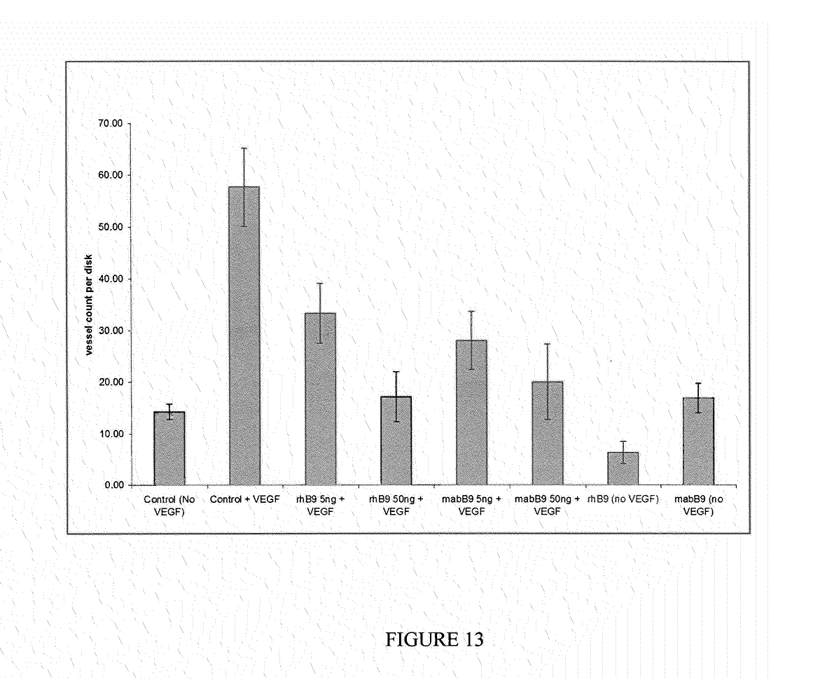

Neither BMP9 nor anti-BMP9 had a substantial effect on angiogenesis in the absence of exogenous VEGF, probably because the lack of angiogenesis in the absence of exogenous VEGF decreases the sensitivity of the assay. See FIG. 13, right hand columns. In the absence of VEGF, both proteins were used at the 50 ng dosed 1×/day on days 1 and 3 in the 5-day cycle. However, in the presence of VEGF, both BMP9 and its antibody had a substantial anti-angiogenic effect. See FIG. 13. These data are consistent with data from Scharpfenecker et al., with respect to BMP9 and VEGF in combination, and are also consistent with the conclusions of Scharpfenecker et al., and David et al., with respect to the anti-angiogenic effects of BMP9 itself. However, the effects of the anti-BMP9 antibody are in remarkable contrast to the published literature. Based on these data, we hypothesize that optimal or physiological levels of BMP9 may be needed for proper angiogenesis, and that either an excess or deficiency in BMP9 will inhibit angiogenesis.

Intriguingly, the effects of the anti-BMP9 antibody are consistent with data presented here showing that ALK1-Fc (which is an alternative BMP9 antagonist) also inhibits angiogenesis. Thus, these data demonstrate that ALK1-Fc and anti-BMP9 each have anti-angiogenic effects, and that anti-BMP9 antibody is likely to be useful in the treatment of angiogenic disorders, such as tumors, rheumatoid arthritis and ocular disorders, in much the same way that ALK1-Fc is shown to be.

Given the anti-angiogenic activity of the MAB3209, we propose that this murine monoclonal antibody could be humanized to provide a therapeutic agent for use in humans. The antibody may be humanized by a variety of art-recognized techniques, including chimerization, CDR-grafting, resurfacing, back mutations, superhumanization, human string content optimization, and empirical methods, such as FR library generation and selection, FR shuffling and humaneering. See, e.g, Almagro and Fransson, Frontiers in Biosciences, 13: 1619-1633, 2008.

### Example 10

### Effects of ALK1-Fc Fusion Protein on Breast Cancer Tumor Models

mALK1-Fc was effective in delaying the growth of breast cancer tumor cell lines derived from both estrogen receptor positive (ER+) and estrogen receptor negative tumor cells (ER−).

The MDA-MB-231 breast cancer cell line (derived from ER− cells) was stably transfected with the luciferase gene to allow for the in vivo detection of tumor growth and potential metastasis. In this study, 1×10⁶ MDA-MB-231-Luc cells were implanted orthotopically in the mammary fat pad of athymic nude mice (Harlan). Tumor progression was followed by bioluminescent detection using an IVIS Spectrum imaging system (Caliper Life Sciences). An increase in the luminescence (number of photons detected) corresponds to an increase in tumor burden.

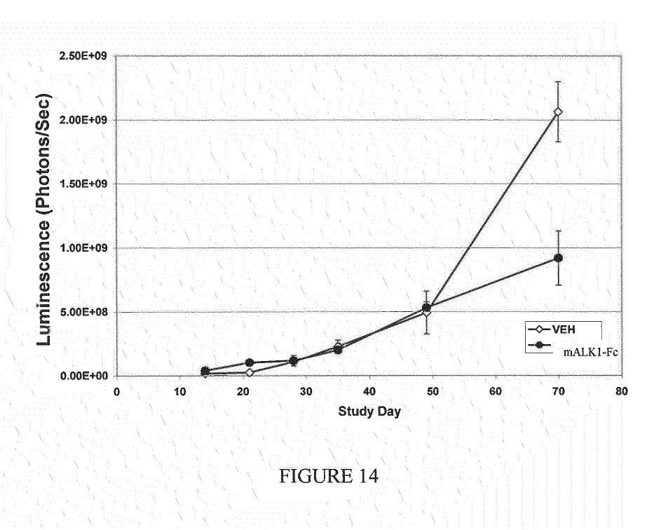

Thirty female nude mice were injected with 1×10⁶ tumor cells into the mammary fat pad. Three days after tumor implantation the mice were treated with either vehicle control or mALK1-Fc (30 mg/kg) twice per week by subcutaneous (SC) injection. Treatment was continued and tumor progression was monitored by bioluminescent imaging for 10 weeks. mALK1-Fc treatment at 30 mg/kg slowed tumor progression as determined by bioluminescent detection when compared to vehicle treated controls (FIG. 14). Treatment with mALK1-Fc delayed, but did not reverse tumor growth in this model. This may be expected of an antiangiogenic compound in that tumors may be able to survive to a certain size before requiring new blood vessel formation to support continued growth. In a similar experiment, hALK1-Fc produced similar, if slightly lesser, effects at dose levels as low as 3 mg/kg.

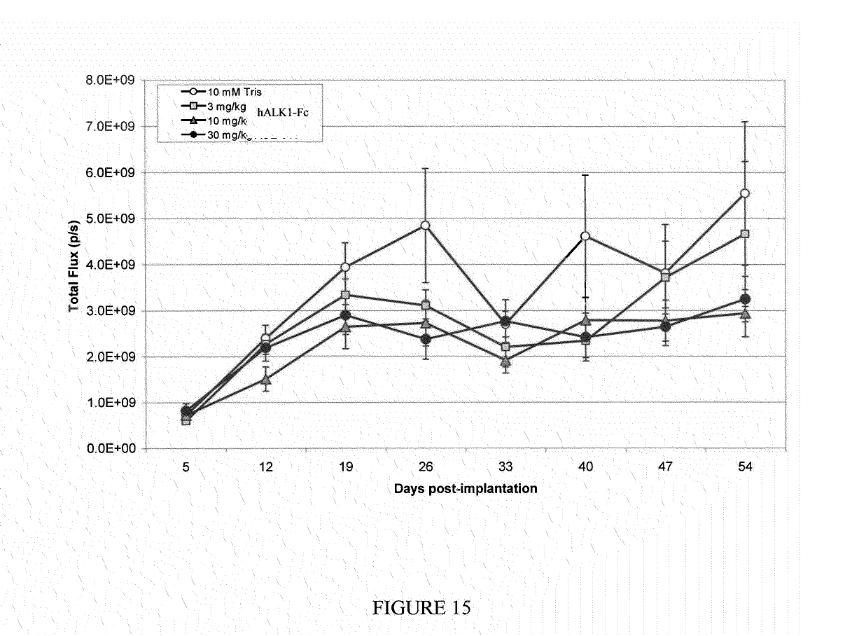

The estrogen-receptor-positive (ER+), luciferase expressing cell line, MCF-7, was also tested in an orthotopic implantation model. In this model, female nude mice are implanted subcutaneously with a 60 day slow release pellet of 17β-estradiol. Two days following pellet implantation, 5×10⁶ MCF-7 tumor cells were implanted into the mammary fat pad. Mice were treated twice per week with hALK1-Fc at 3, 10 and 30 mg/kg, or vehicle control, by the IP route. Tumor progression was followed by bioluminescent imaging on a weekly basis with an IVIS-Spectrum imager (Caliper Life Sciences). In vehicle treated mice tumors progressed rapidly until study day 26 (FIG. 15). After day 26, there were fluctuations in tumor luminescence until the conclusion of the study at day 60 (when the estradiol pellets were depleted). These fluctuations are due to a common feature of this model in that the rapid tumor growth can exceed the angiogenic response of the host animals leading to tumor necrosis and a concomitant drop-off in luminescent signal. The remaining cells continue to grow leading to an increased signal. Mice treated with 10 or 30 mg/kg of hALK1-Fc were able to maintain tumor size at a constant level during the study, compared to vehicle-treated controls, indicating a potent effect of this molecule on tumor growth.

### Example 11

### Inhibition of BMP10 Signaling by hALK1-Fc in a Cell-Based Assay

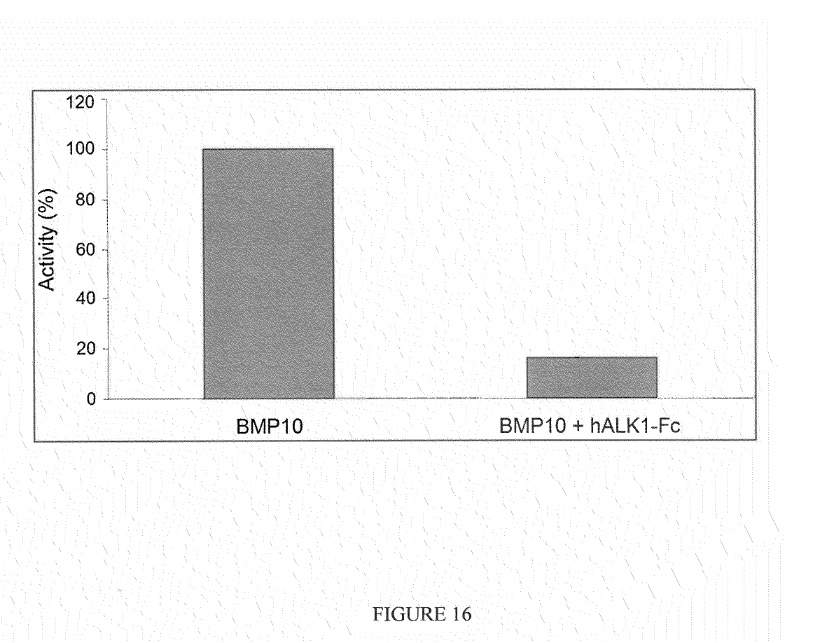

Effects of hALK-Fc on BMP10 signaling were determined in a cell-based assay, in which human glioblastoma T98G cells were transfected with three plasmids: 1) an expression construct encoding full-length ALK1; 2) a firefly-luciferase reporter construct (see Example 3) responsive to Smad1/5/8-mediated signaling, and 3) a Renilla-luciferase control construct. Treatment of transfected cells with recombinant human BMP10 (1 ng/ml) strongly stimulated firefly luciferase activity relative to Renilla luciferase activity (FIG. 16). Omission of the ALK1 expression construct reduced BMP10-stimulated activity by approximately two-thirds (data not shown), thus implicating ALK1 as a major mediator of the BMP10 signal. Treatment of fully transfected cells with hALK1-Fc (65 ng/ml) and BMP10 (1 ng/ml) reduced the transcriptional response compared to BMP10 alone by more than 80% (FIG. 16). Together, these results indicate that ALK1 is a major mediator of BMP10 signaling and that ALK1-Fc can markedly inhibit such signaling.

### Example 13

### ALK-Fc Enhances the Activity of Sunitinib in the 786-0 Tumor Xenograft Model

786-0 cells, a von Hippel Lindau (VHL)-deficient human renal cell carcinoma (RCC) cell line (see Iliopoulos et al., Nature Medicine 1995; 1:822-6), was obtained from the American Type Culture Collection, and cultured in RPMI 1640 medium (Cellgro). Media was supplemented with 2 mmol/L L-glutamine, 10% FCS, and 1% streptomycin (50 μg/mL), and cells were cultured at 37° C. with 5% CO₂. 786-0 cells were harvested from subconfluent cultures by a brief exposure to 0.25% trypsin and 0.02% EDTA. Trypsinization was stopped with medium containing 10% fetal bovine serum and the cells were washed once in serum-free medium and resuspended in PBS. Only suspensions consisting of single cells with >90% viability were used for the injections.

To establish human RCC xenografts, 786-0 tumor cells were injected subcutaneously (1×10⁷ cells) into the flanks of 6- to 8-week-old female athymic nude/beige mice (Charles River Laboratories) that were of 20 g average body weight. Tumors developed in >80% of the mice and were usually visible within a few days of implantation. Mice were treated with vehicle plus Fc, sunitinib plus Fc, vehicle plus ALK-Fc, or sunitinib plus ALK-Fc when the tumors had grown to a diameter of 12 mm. Sunitinib (53.6 mg/kg; Pfizer) was administered 6 of 7 days per week by gavage beginning. ALK1-Fc (10 mg/kg) was administered 3 times per week intraperitoneally. Tumors were measured every two days while mice were on treatment.

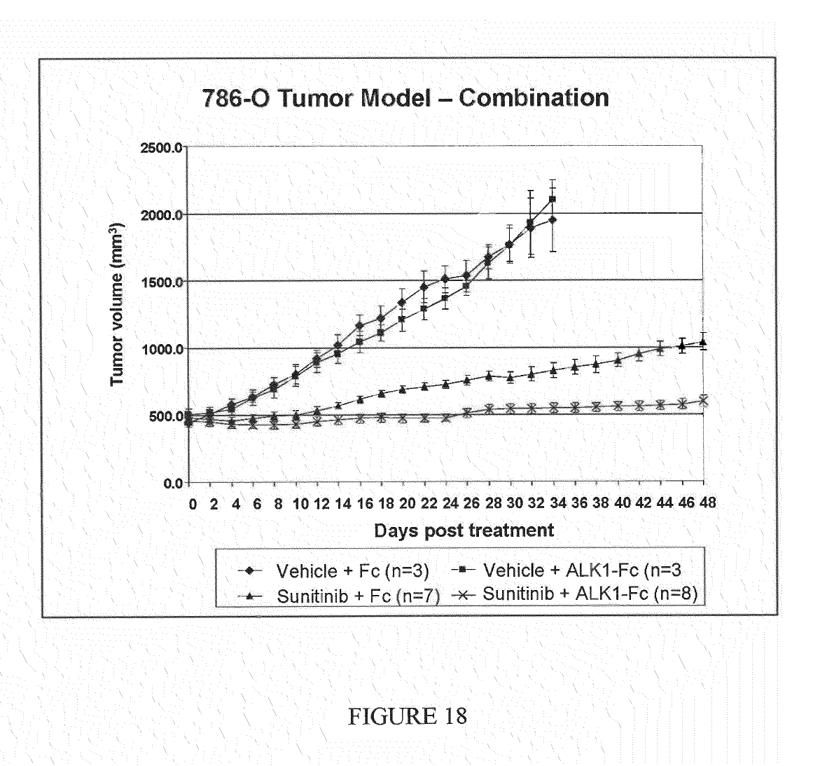

As shown in FIG. 18, treatment with sunitinib plus Fc slowed tumor growth in the 786-0 murine human tumor xenograft model. This effect was further enhanced when tumors were treated with sunitinib plus ALK-Fc indicating that ALK-FC enhances the tumor growth inhibiting activity of sunitinib in a model for clear cell renal cell carcinoma.

### Example 14

### ALK-Fc has Single Agent Activity in the A498 Tumor Xenograft Model

A498 cells, a VHL-deficient human RCC cell line (see Iliopoulos et al., Nature Medicine 1995; 1:822-6), was obtained from the American Type Culture Collection, and cultured in Eagle's minimal essential medium. Media was supplemented with 2 mmol/L L-glutamine, 10% FCS, and 1% streptomycin (50 μg/mL), and cells were cultured at 37° C. with 5% CO₂. 786-0 cells were harvested from subconfluent cultures by a brief exposure to 0.25% trypsin and 0.02% EDTA. Trypsinization was stopped with medium containing 10% fetal bovine serum, and the cells were washed once in serum-free medium and resuspended in PBS. Only suspensions consisting of single cells with >90% viability were used for the injections.

To establish human RCC xenografts, A498 tumor cells were injected subcutaneously (1×10⁷ cells) into the flanks of 6- to 8-week-old female athymic nude/beige mice (Charles River Laboratories) that were of 20 g average body weight. Tumors developed in >80% of the mice and were usually visible within a few days of implantation. Mice were treated with Fc or ALK-Fc when the tumors had grown to a diameter of 12 mm. ALK-Fc (10 mg/kg) was administered 3 times per week intraperitoneally. Tumors were measured daily while mice were on treatment.

As shown in FIG. 19, ALK-FC has single agent activity as treatment with ALK-Fc alone dramatically slowed tumor growth in the A498 murine human tumor xenograft model.

### Example 15

### ALK-Fc also Enhances the Activity of Sunitinib in the A498 Tumor Xenograft Model

A498 cell culture and xenograft establishment was performed as described in Example 14. Mice were treated with vehicle plus Fc, sunitinib plus Fc, vehicle plus ALK-Fc, or subitinib plus ALK-Fc when the tumors had grown to a diameter of 12 mm. Sunitinib (53.6 mg/kg; Pfizer) was administered 6 of 7 days per week by gavage beginning. ALK1-Fc (10 mg/kg) was administered 3 times per week intraperitoneally. Tumors were measured daily while mice were on treatment.

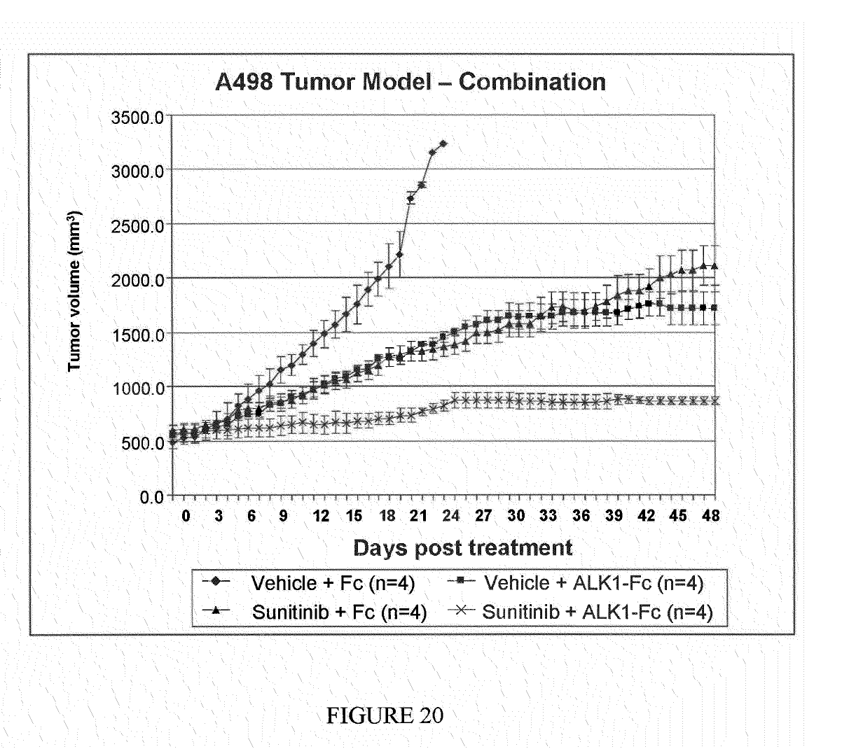

As shown in FIG. 20, treatment with sunitinib plus Fc or vehicle plus ALK-Fc slowed tumor growth in the A498 tumor xenograft model. However, when administered in combination, ALK-FC substantially increased the tumor growth inhibiting activity of sunitinib on A498 tumor growth.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject inventions are explicitly disclosed herein, the above specification is illustrative and not restrictive. Many variations of the inventions will become apparent to those skilled in the art upon review of this specification and the claims below. The full scope of the inventions should be determined by reference to the claims, along with their full scope of equivalents, and the specification, along with such variations.

1. A method of treating renal cell carcinoma (RCC) in a mammal, comprising administering to a mammal that has RCC an effective amount of a receptor tyrosine kinase inhibitor (RTKI) and an agent selected from:
(a) an ALK1 polypeptide comprising a ligand binding portion of the extracellular domain of ALK1;
(b) an anti body that hinds to the extracellular domain of human ALK1;
(c) an antibody that binds to human BMP9; and
(d) an antibody that binds to human BMP10.

2. The method of claim 1, wherein the ALK1 polypeptide comprises a polypeptide having, an amino acid sequence that is at least 90% identical to the sequence of amino acids 22-118 of SEQ ID NO: 1, and wherein the ALK1 polypeptide binds to an ALK1 ligand selected from GDF5, GDF6, GDF7, BMP9 and BMP10.

3. The method of claim 2, wherein the ALK1 polypeptide comprises a polypeptide having an amino acid sequence that is at least 90% identical to the sequence of amino acids 22-120 of SEQ ID NO:1.

4. The method of claim 2, wherein the ALK1 polypeptide further comprises a constant domain of an immunoglobulin.

5. The method of claim 2, wherein the ALK1 polypeptide further comprises an Fc portion of an immunoglobulin.

6. The method of claim 5, wherein the Fc portion is an Fc portion of a human IgG1.

7. The method of claim 1, wherein the ALK1 polypeptide comprises an amino acid sequence that is at least 90% identical to the sequence of SEQ ID NO: 3 or SEQ ID NO:14.

8. The method of claim 1, wherein the antibody of (b) binds to an epitope within the sequence of amino acids 22-118 of SEQ ID NO:1 and inhibits binding of a ligand selected from GDF5, GDF6, GDF7, BMP9 and BMP 10.

9. The method of claim 1, wherein the antibody of (c) binds to an epitope within the sequence of amino acids 1-111 of SEQ ID NO:12 and inhibits binding of BMP9 to a receptor.

10. The method of claim 1, wherein the antibody of (d) binds to an epitope within the sequence of amino acids 1-108 of SEQ ID NO:13 and inhibits binding of BMP10 to a receptor.

11. The method of claim 1, wherein the RTKI is sunitinib.

12. The method of claim 1, wherein the RTKI sorafenib.

13. The method of claim 1, wherein the RTKI is pazopanib.

14. The method of claim 1, wherein the RTKI is axitinib.

15. The method of claim 1, wherein the RTKI is tivozanib or vandetanib.

16. The method of claim 1, which further comprises administering a mammalian target of rapamycin (mTOR)-targeted inhibitor.

17. The met method of claim 6, wherein the mTOR-targeted inhibitor is everolimus.

18. The method of claim 16, wherein the mTOR-targeted inhibitor is temsirolimus.

19. The method of claim 1, wherein the RCC is a clear cell renal cell carcinoma.

20. The method of claim 1, wherein the RCC has invaded the renal sinus.

21. The method of claim 1, wherein the RCC is metastatic RCC.

22. The method of claim 1, wherein the RCC has metastasized to the lung, intra-abdominal lymph nodes, bone, brain, or liver.

23. A method of treating renal cell carcinoma in a mammal having previously received an RCC therapeutic agent, the method comprising administering to the mammal an effective amount of an agent selected from:
(a) an ALK1 polypeptide comprising a ligand binding portion of the extracellular domain of ALK1;
(b) an antibody that binds to the extracellular domain of human ALK1;
(c) an antibody that binds to man BMP9; and
(d) an antibody that binds to human BMP10.

24. The method of claim 23, wherein the ALK1 polypeptide comprises a polypeptide having an amino acid sequence that is at least 90% identical to the sequence of amino acids 22-118 of SEQ ID NO: 1, and wherein the ALK1 polypeptide binds to an ALK1 ligand selected from GDF5, GDF6, GDF7, BMP9 and BMP 10.

25. (canceled)

26. The method of claim 24, wherein the ALK1 polypeptide further comprises a constant domain of an immunoglobulin.

27. The method of claim 24, wherein the ALK1 polypeptide farther comprises an Fc portion of an immunoglobulin.

28. The method of claim 27, wherein the Fc portion is an Fc portion of a human IgG1.

29. The method of claim 23, wherein the ALK1 polypeptide comprises an amino acid sequence that is at least 90% identical to the sequence of SEQ ID NO: 3 or SEQ ID NO:14.

30. The method of claim 23, wherein the antibody of (b) hinds to an epitope within the sequence of amino acids 22-118 of SEQ ID NO:1 and inhibits binding of a ligand selected from GDF5, GDF6, GDF7, BMP9 and BMP 10.

31. The method of claim 23, wherein the antibody of (c) binds to an epitope within the sequence of amino acids 1-111 of SEQ ID NO:12 and inhibits binding of BMP9 to a receptor.

32. The method of claim 23, wherein the antibody of (d) binds to an epitope within the sequence of amino acids 1-108 of SEQ ID NO:13 and inhibits binding of BMP10 to a receptor.

33. The method of claim 23, wherein the previously received RCC therapeutic agent is an RTKI.

34. The method of claim 33, wherein the RTKI is selected from: sunitinib, sorafenib, pazopanib, axitinib, tivozanib and vandetanib.

35. The method of claim 23, wherein the previously received RCC therapeutic agent is a mammalian target of rapamycin (mTOR)-targeted inhibitor.

36. The method of claim 35, wherein the mTOR-targeted inhibitor is an agent selected from: everolimus and temsirolimus.

37. The method of claim 23, wherein the previously received therapeutic agent is interferon alpha (IFN-alpha) or interleukin-2 (IL-2).

38. The method of claim 23, which further comprises administering an RTKI.

39. The method of claim 38, wherein the RTKI is an agent selected from: sunitinib, sorafenib, pazopanib axitinib, tivozanib and vandetanib.

40. The method of any of claim 23, which further comprises administering an mTOR targeted inhibitor.

41. The method of claim 40, wherein the mTOR-targeted inhibitor is an agent selected from everolimus and temsirolimus.

42. The method of claim 23, wherein the RCC is a clear cell renal cell carcinoma.

43. The method of claim 42, wherein the RCC has invaded the renal sinus.

44. The method of claim 23, wherein the RCC is metastatic RCC.

45. The method of claim 23, wherein the RCC has metastasized to the lung, intra-abdominal lymph nodes, bone, brain, or liver.
